(19) United States
(12) Patent Application Publication
(10) Pub. No.: US 2013/0039956 A1
(43) Pub. Date: Feb. 14, 2013

(54) USE OF NITROCARBOXYLIC ACIDS FOR THE TREATMENT, DIAGNOSIS AND PROPHYLAXIS OF AGGRESSIVE HEALING PATTERNS

(71) Applicant: Ulrich Dietz, Wiesbaden (DE)

(72) Inventor: Ulrich Dietz, Wiesbaden (DE)

(73) Assignee: Ulrich DIETZ, Wiesbaden (DE)

(21) Appl. No.: 13/634,949

(22) PCT Filed: Feb. 1, 2011
(86) PCT No.: PCT/EP2011/000429

§ 371 Date: Oct. 23, 2012

Related U.S. Application Data
(60) Provisional application No. 61/282,675, filed on Mar. 15, 2010.

(51) Int. Cl. A61K 9/00; A01N 1/02; A61P 29/00; A61P 25/04; A61P 17/02; A61K 31/22; A61K 31/7076

(52) U.S. Cl. 424/400; 514/551; 435/1.1; 514/47

(57) ABSTRACT

The invention is directed to implants and medical devices having at least one layer which contains at least one nitrocarboxylic acid. These implants and medical devices shall be used for the prophylaxis and treatment of aggressive healing patterns. Furthermore, this invention relates to the use of nitrocarboxylic acids and their pharmaceutically acceptable salts as a therapeutic agent for the prophylaxis and treatment of a pathophysiological or non-physiological healing pattern due to exposure to a physical, chemical or thermal irritant of tissues, cells or organelles.

Every cell of an organism reacts to external influences through a multitude of molecular mechanisms and structural changes. Thus, genes can be activated which produce a change in cell metabolism, phenotype, expression of membrane receptors, membrane functionality and release of molecules and vesicles which then initiate a local or a systemic reaction. The magnitude, respectively the degree of the cellular response is generally correlated with the magnitude of cell damage. The damage can be caused by ionization, the exceeding or dropping below of a critical temperature as well as of a critical pH value, osmotic pressure or electrolyte concentration, by toxins, detergents, mechanical injuries, exposure to tensile or shear forces, exceeding or falling below a critical pressure (barotrauma), etc. The degree of injury of the individual cell or a group of cells determines the extent of the cellular response, respectively the response pattern. These response patterns can (1) have minimal consequences, such as opening of intercellular tight junctions, (2) result in a regionally limited effect, such as producing extracellular matrix compounds, as well as local and remote reactions, e.g., local fibrin adhesion and the release of microparticles for the recruitment of progenitor cells from bone marrow, or (3) cause complex local and systemic reactions, which can activate the complete immune system of the organism. The goal of these response patterns is to re-establish cellular integrity, which is also known as healing. The healing process can be divided into three, simplified response patterns: (1) passive, i.e., altered cell functions and cell morphology are completely re-established without change of tissue texture or function, (2) an active healing process that has the function of repairing or refilling damaged or destroyed structures, e.g., formation of an extracellular matrix to fill defects as well as decomposition of cell debris, cell mitosis with contact inactivation, and (3) an aggressive healing process, i.e., formation of extracellular matrix as well as cell proliferation, which goes beyond the amount of material needed to fill the defect. An aggressive healing process can occur, when cell damage continues, e.g., persisting exposure to tensile or shear forces, toxins as well as chemical irritations or via extensive tissue damage or bacterial colonization.

The passive healing process leads to a restitutio ad integrum, i.e., functional or structural changes do not occur.

Active healing is a healing process that, as a rule, maintains the functionality of the tissue by restoring its integrity. However, the texture of newly formed tissues can differ from that before wounding/trauma which does not cause mal- or dysfunction of the affected organ/structure, nor esthetic or cosmetic impairments.

In contrast, aggressive healing processes lead to either functional or structural dysfunction of the tissue or the affected organ as well as to aesthetic problems, which require further medicinal treatments/measures. An aggressive healing process may lead to adverse side effects of the causative alteration and/or a therapeutic measure, such as fusion by tight adhesion of connective tissue layers or increased tissue stiffness. Massive adhesions of tissue layers often render renewed surgical access difficult, or due to adhesions functional disorders can result in the same or in another organ. In addition, increased tissue stiffness can cause functional disorders or cosmetic impairment. In the case of vasculopathy, this can lead to a decreased blood supply of the organ.

The exact conditions that cause an active or aggressive healing pattern are still not known. However, many medical conditions are known to have an inherent risk for developing an aggressive healing pattern.

It is known that cells can react differently to the same stimuli/irritation and that this plasticity can be influenced by external and internal measures. Several known response patterns as well as their ability to be influenced are described in the following.

Cells have numerous sensors which can perceive most cell-damaging stimuli or irritants. In one aspect this applies to the perception of shear forces. Many cells alter their phenotype as a reaction to the activation of these sensors which can result in further changes in metabolism occurring in parallel. It could be shown that subtle mechanical alterations are responsible for this reaction. The perception of the mechanical impulses to the cytosceleton is, however, influenced by the cell wall components or by the physical characteristics of the cell membrane itself.

A further aspect that can cause an aggressive healing pattern is a concomitant inflammation while a tissue healing process takes place. This can be explained by a simultaneous activation of cell signaling pathways which may arise during the course of the healing process and by the inflammatory process. However, an inflammation does not lead to an aggressive healing pattern by itself. There are innumerous clinical situations/diseases classified as an inflammation by medical textbooks that completely resolve without any damage/dysfunction of the affected tissues/organs such as pneumonia, gastritis, osteomyelitis caused by bacteria, viruses or microbes. Furthermore, an inflammation is clinically characterized by the coincidence of several pathological changes leading locally to hyperemia and edema as well as to a recruitment of local and systemic defense systems which induce an infiltration of white blood cells (leukocytes). An invasion of macrophages, however, can also be seen in an active healing pattern in order to remove cell fragments, thereby not causing an inflammatory process.

Although an inflammatory process can be involved in an aggressive healing pattern, the characteristic changes occurring during aggressive healing—such as dedifferentiation, migration and division of endothelial and mesenchymal cells as well as of fibroblasts which in addition produce extracellular matrix—can be caused by numerous conditions which can not be summarized under the term inflammation. This is underlined by the fact that stimulating mediators are produced by various cell types and even by the affected cells via autokrine loop stimulation. A classical example is the reactive process of the left ventricular wall as a consequence of increased blood pressure which causes hypertrophy accompanied by fibrotic changes of the tissue texture without involvement of white blood cells. Another textbook example is a change in intracellular and/or extracellular pH. An inflammation generally entails an acidosis in the affected tissue. But not every pH shift in the tissue is due to an inflammation, respectively the recovery from an inflammation. It may occur in many other diseases or states, such as gastric ulcer, stroke or epileptic seizure.

Severe traumatisation of cells, organelles or tissues can lead to an inflammatory response, which in turn may reinforce cell, organelle or tissue damage as well as induce an aggressive healing pattern. However, blocking a single or multiple key pathways of inflammatory signal transduction reduces, but does not inhibit the inflammatory response to a trauma. Therefore effects on inflammatory pathways by nitro-fatty acids can not explain inventive actions on the response to irritation, trauma or damage from the cells, organelles or tissues. The stabilisation of the membranes themselves or of their constituents was hypothesized as the mechanism of action which leads to a different reaction pattern of the irritated cell, organelle or tissue. In other words, nitrocarboxylic acids incorporated into those membranes render them more resistant to physical, chemical or electrical irritations, thus modulating the cell, organelle or tissue response to them. This may lead to an attenuation of the cell, organelle or tissue damage resulting from an irritation. Moreover, initiation of components of the healing (repair) process are initialized by mediators like transforming growths factor β-1 and IGFBP-5 [IGF (insulin-like growth factor)-binding protein-5] (Allan et al., J Endocrinol 2008, 199, 155-164; Sureshbabu et al., Biochem Soc Trans 2009, 37, 882-885). The release of the fibroblast stimulating mediators is controlled by integrins as a respond to various cell stress factors (Wipff et al., Eur J Cell Biol 2008, 87, 601-615). Furthermore, cell membrane receptors such as Angiotensin II-1 and Plasminogen activator inactivator-1 (PAI-1) receptor are expressed which could mediate migratory and/or mitotic responses (Pedroja et al., J Biol Chem 2009, 284, 20708-20717; de Cavanagh et al., Am J Physiol Heart Circ Physiol 2009, 296, H550-558). Moreover, the existence of an angiotensin/TGF-beta1 “autocrine loop” in human lung myofibroblasts was proposed (Uhal et al., Curr Pharm Des 2007, 1, 1247-1256). This was found to apply also for burn injuries (Gabriel et al., J Burn Care Res 2009, 30, 471-481). In other words, this reaction cascade as a response to injury enables the cell itself and the neighboring cells to react by changing their morphology, by migration, by cell division or by the production of extracellular matrix compounds. It could be shown that stimulation of quiescent ceratocyts or fibroblasts results in fibrosis.

In order to delineate an inflammation as the pathophysiological cause for development of an aggressive healing pattern from other causes in which nitrocarboxylic acids are claimed to effecticely prevent or treat an aggressive healing pattern, at least three key features (as defined below) must coincide before a disease or state can be properly addressed as a genuine inflammation. All other clinical conditions/diseases which do not involve a genuine inflammation or in which inflammatory features are of inferior relevance can be called as non-inflammatory. This view is further encouraged by scientific evidence that blocking of one or more mediator of an inflammation by pharmacological intervention can't prevent an aggressive wound healing in general. This holds also true for various physiological (e.g., glucocorticoids) or pharmaceutical (cytocine antibodies) substances that were shown to have anti-inflammatory or anti-proliferative effects.

This holds also true for the inhibition of various cell signal pathways which mediate an inflammatory stimulus.

Perception und signal transduction of a cell is largely controlled by physical and physicochemical properties of the cell membrane.

An activation of the peroxisome proliferator-activated receptors (PPAR) or stimulation of hemoxygenase-1 production has been found to reduce cell proliferation in several cell culture models; however, a significant inhibition of pathological healing processes could not be confirmed in clinical settings.

The influence of nitrocarboxylic acids on cellular membranes has not yet been studied. Surprisingly, the inventive nitrocarboxylic acids were found to have—most probably unspecific—effects on the physicochemical properties of cell and organelle membranes that result in alterations of cell perception and signal transduction of various membrane proteins/constituents, thus tuning cell responsiveness to environmental influences. This could be used to modify the responsiveness of cells or organelles involved in an alteration/injury/trauma, thus preventing or reducing an aggressive healing response.

This effect of nitrocarboxylic acids cannot be explained by hitherto known mechanisms on the intracellular reaction pathways that have been documented for nitrocarboxylic acids or by their combined inhibition or stimulation. Moreover, the therapeutic uptake of nitrocarboxylic acids into cell membranes results in a complex inhibition of the transmission of the cellular damage inside and outside the cell, so that the internal and external cell response pathways are not initiated or activated.

Nitrocarboxylic acids have so far not been tested for an anesthetic effect. Surprisingly, a reduction in the perception of pain could be achieved by the topical application of nitrocarboxylic acids. An inhibition of pain perception is presumably responsible for this phenomenon because the release and re-uptake of neurotransmitters in the synaptic cleft is influenced by the membrane composition. These effects cannot be explained by the influence of nitrocarboxylic acids on distinct cell signal pathways or their combined activation or inhibition. Thus, the use of the nitrocarboxylic acids according to the invention for the effects described above represents an innovative prophylactical and therapeutic concept.

Thus the objective of the present invention is to find compounds which are able to inhibit an aggressive healing pattern. Thus the objective is solved by the ensuing technical teachings of the independent claims of the present invention. Further advantageous embodiments of the invention result from the dependent claims, the description and the examples.

Surprisingly, it was found that this objective can be solved by the use of nitrocarboxylic acids for the therapy and prophylaxis of such diseases in which such an aggressive healing pattern is involved. Surprisingly, it was also found that a coating of implants and medical devices with nitrocarboxylic acids (herein also referred to as nitrated fatty acids) is particularly advantageous for the healing process to avoid aggressive healing patterns, even at subthreshold concentrations at which no pharmacological action is to be expected.

The mechanism of action involves the modulation of the response of membranes from cells or organells to an irritation/stimulus potentially causing a pathological or non-physiologic reaction including cell degranulation, cell dedifferention, cell migration, cell division, production of extracellular matrix, foreign body formation, and cell death. An additional prophylactical and therapeutical effect is the stabilization of cell membrane properties (resilience against mechanical, chemical or electrical irritations) and functionality (membrane potential, regulation of ion channels, transmembrane signal transduction). Furthermore, these compounds shall attenuate symptoms which may occur in diseases in which such an aggressive healing pattern is involved.

### DESCRIPTION

Surprisingly, it was found that nitrocarboxylic acids of the general formula (X)

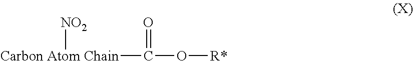

can be used for the treatment or prophylaxis of a disease or a state displaying an aggressive healing response of tissues, cells or organelles in a mammal including humans and can also be used for the manufacture of a pharmaceutical composition or of a composition for a passive coating for the treatment or prophylaxis of a disease or a state displaying an aggressive healing response of tissues, cells or organelles.

Such diseases or states are displaying an aggressive healing response which results from an exogenous irritation, wounding or trauma, wherein the disease or state in which such an exogenous irritation, wounding or trauma occurs is selected from burn, chemical burn, alkali burn, burning, hypothermia, frostbite, cauterization, granuloma, necrosis, ulcer, fracture, foreign body reaction, cut, scratch, laceration, bruise, tear, contusion, fissuring or burst. Moreover such diseases or states result from an endogenous irritation or stimulation by acute or chronical physical, chemical or electrical means. Examples for diseases or states in which such an endogenous irritation or stimulation occurs are fascitis, tendonitis, neuropathy, or prostate hypertrophy.

In formula (X) the residue R* represents hydrogen, a polyethylene glycol residue, a polypropylene glycol residue, cholesteryl, phytosteryl, ergosteryl, a coenzyme A residue or an alkyl group consisting of 1 to 10 carbon atoms, preferably 1 to 7 carbon atoms, wherein this alkyl group may contain one or more double and/or one or more triple bonds, may be cyclic and/or may be substituted by one or more nitro groups and/or one or more substituents S¹-S²⁰.

The term “nitrocarboxylic acid” refers also to nitrocarboxylic acid esters. Thus the term “nitrocarboxylic acid” explicitly covers also these compounds wherein R* is not hydrogen, namely the esters of the nitrocarboxylic acids. Consequently, everywhere where the term “nitrocarboxylic acid” is used, also the corresponding esters are meant which are represented by the general formula (X) wherein R* is not H. Preferably R* represents one of the following substituents: —CH₂F, —CHF₂, CF₃, —CH₂Cl, —CH₂Br, —CH₂I, —CH₂CH₂F, —CH₂CHF₂, —CH₂CF₃, —CH₂CH₂Cl, —CH₂CH₂Br, —CH₂CH₂I, cyclo-C₃H₅, cyclo-C₄H₇, cyclo-C₅H₉, cyclo-C₆H₁₁, cyclo-C₇H₁₃, cyclo-C₈H₁₅, -Ph, —CH₂-Ph, —CPh₃, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₇H₁₅, —C₈H₁₇, —C₉H₁₉, —C₁₀H₂₁, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃, —CH═CH₂, —CH₂—CH═CH₂, —C(CH₃)═CH₂, —CH═CH—CH₃, —C₂H₄—CH═CH₂, —CH₂—CH═CH—CH₃, —CH═CH—C₂H₅, —CH₂—C(CH₃)═CH₂, —CH(CH₃)—CH═CH, —CH═C(CH₃)₂, —C(CH₃)═CH—CH₃, —CH═CH—CH═CH₂, —C₃H₈—CH═CH₂, —C₂H₄—CH═CH—CH₃, —CH₂—CH═CH—C₂H₅, —CH═CH—C₃H₇, —CH₂—CH═CH—CH═CH₂, —CH═CH—CH═CH—CH₃, —CH═CH—CH₂—CH═CH₂, —C(CH₃)═CH—CH═CH₂, —CH═C(CH₃)—CH═CH₂, —CH═CH—C(CH₃)═CH₂, —C₂H₄—C(CH₃)═CH₂, —CH₂—CH(CH₃)—CH═CH₂, —CH(CH₃)—CH₂—CH═CH₂, —CH₂—CH═C(CH₃)₂, —CH₂—C(CH₃)═CH—CH₃, —CH(CH₃)—CH═CH—CH₃, —CH═CH—CH(CH₃)₂, —CH═C(CH₃)—C₂H₅, —C(CH₃)═CH—C₂H₅, —C(CH₃)═C(CH₃)₂, —C(CH₃)₂—CH═CH₂, —CH(CH₃)—C(CH₃)═CH₂, —C(CH₃)═CH—CH═CH₂, —CH═C(CH₃)—CH═CH₂, —CH═CH—C(CH₃)═CH₂, —C₄H₈—CH═CH₂, —C₃H₆—CH═CH—CH₃, —C₂H₄—CH═CH—C₂H₅, —CH₂—CH═CH—C₃H₇, —CH═CH—C₄H₉, —C₃H₆—C(CH₃)═CH₂, —C₂H₄—CH(CH₃)—CH═CH₂, —CH₂—CH(CH₃)—CH₂—CH═CH₂, —CH(CH₃)—C₂H₄—CH═CH₂, —C₂H₄—CH═C(CH₃)₂, —C₂H₄—C(CH₃)═CH—CH₃, —CH₂—CH(CH₃)—CH═CH—CH₃, —CH(CH₃)—CH₂—CH═CH—CH₃, —CH₂—CH═CH—CH(CH₃)₂, —CH₂—CH═C(CH₃)—C₂H₅, —CH₂—C(CH₃)═CH—C₂H₅, —CH(CH₃)—CH═CH—C₂H₅, —CH═CH—CH₂—CH(CH₃)₂, —CH═CH—CH(CH₃)—C₂H₅, —CH═C(CH₃)—C₃H₇, —C(CH₃)═CH—C₃H₇, —CH₂—CH(CH₃)—C(CH₃)═CH₂, —CH(CH₃)—CH₂—C(CH₃)═CH₂, —CH(CH₃)—CH(CH₃)—CH═CH₂, —CH₂—C(CH₃)₂—CH═CH₂, —C(CH₃)₂—CH₂—CH═CH₂, —CH₂—C(CH₃)═C(CH₃)₂, —CH(CH₃)—CH═C(CH₃)₂, —C(CH₃)₂—CH═CH—CH₃, —CH(CH₃)—C(CH₃)═CH—CH₃, —CH═C(CH₃)—CH(CH₃)₂, —C(CH₃)═CH—CH(CH₃)₂, —C(CH₃)═C(CH₃)—C₂H₅, —CH═CH—C(CH₃)₃, —C(CH₃)₂—C(CH₃)═CH₂, —CH(C₂H₅)—C(CH₃)═CH₂, —C(CH₃)(C₂H₆)—CH═CH₂, —CH(CH₃)—C(C₂H₆)═CH₂, —CH₂—C(C₃H₇)═CH₂, —CH₂—C(C₂H₅)═CH—CH₃, —CH(C₂H₆)—CH═CH—CH₃, —C(C₄H₆)═CH₂, —C(C₃H₇)═CH—CH₃, —C(C₂H₅)═CH—C₂H₅, —C(C₂H₅)═C(CH₃)₂, —C[C(CH₃)₃]═CH₂, —C[CH(CH₃)(C₂H₅)]═CH₂, —C[CH₂—CH(CH₃)₂]═CH₂, —C₂H₄—CH═CH—CH═CH₂, —CH₂—CH═CH—CH₂—CH═CH₂, —CH═CH—C₂H₄—CH═CH₂, —CH₂—CH═CH—CH═CH—CH₃, —CH═CH—CH₂—CH═CH—CH₃, —CH═CH—CH═CH—C₂H₅, —CH₂—CH═CH—C(CH₃)═CH₂, —CH₂—CH═C(CH₃)—CH═CH₂, —CH₂—C(CH₃)═CH—CH═CH₂, —CH(CH₃)—CH═CH—CH═CH₂, —CH═CH—CH₂—C(CH₃)═CH₂, —CH═CH—CH(CH₃)—CH═CH₂, —CH═C(CH₃)—CH₂—CH═CH₂, —C(CH₃)═CH—CH₂—CH═CH₂, —CH═CH—CH═C(CH₃)₂, —CH═CH—C(CH₃)═CH—CH₃, —CH═C(CH₃)—CH═CH—CH₃, —C(CH₃)═CH—CH═CH—CH₃, —CH═C(CH₃)—C(CH₃)═CH₂, —C(CH₃)═CH—C(CH₃)═CH₂, —C(CH₃)═C(CH₃)—CH═CH₂, —CH═CH—CH═CH—CH═CH₂, —C≡C—CH₃, —CH₂—C≡CH, —C₂H₄—C≡CH, —CH₂—C≡C—CH₃, —C≡C—C₂H₅, —C₃H₆—C≡CH, —C₂H₄—C≡C—CH₃, —CH₂—C≡C—C₂H₅, —C≡C—C₃H₇, —CH(CH₃)—C≡CH, —C≡C—C₄H₉, —CH₂—CH(CH₃)—C≡CH, —CH(CH₃)—CH₂—C≡CH, —CH(CH₃)—C≡C—CH₃, —C₄H₈—C≡CH, —C₃H₆—C≡C—CH₃, —C₂H₄—C═C—C₂H₅, —CH₂—C≡C—C₃H₇, —C₂H₄—CH(CH₃)—C≡CH, —CH₂—CH(CH₃)—CH₂—C≡CH, —CH(CH₃)—C₂H₄—C≡CH, —CH₂—CH(CH₃)—C≡C—CH₃, —CH(CH₃)—CH₂—C═C—CH₃, —CH(CH₃)—C≡C—C₂H₅, —CH₂—C≡C—CH(C H₃)₂, —C≡C—CH(CH₃)—C₂H₅, —C≡C—CH₂—CH(CH₃)₂, —C≡C—C(CH₃)₃, —CH(C₂H₅)—C≡C—CH₃, —C(CH₃)₂—C≡C—CH₃, —CH(C₂H₅)—CH₂—C≡CH, —CH₂—CH(C₂H₅)—C≡CH, —C(CH₃)₂—CH₂—C≡CH, —CH₂—C(CH₃)₂—C≡CH, —CH(CH₃)—CH(CH₃)—C≡CH, —CH(C₃H₇)—C≡CH, —C(CH₃)(C₂H₅)—C≡CH, —C≡C—C≡CH, —CH₂—C≡C—C≡CH, —C≡C—C≡C—CH₃, —CH(C≡CH)₂, —C₂H₄—C≡C—C≡CH, —CH₂—C≡C—CH₂—C≡CH, —C═C—C₂H₄—C≡CH, —CH₂—C≡C—C≡C—CH₃, —C≡C—CH₂—C≡C—CH₃, —C≡C—C≡C—C₂H₅, —C≡C—CH(CH₃)—C≡CH, —CH(CH₃)—C≡C—C≡CH, —CH(C≡CH)—CH₂—C≡CH, —C(C≡CH)₂—CH₃, —CH₂—CH(C≡CH)₂, —CH(C≡CH)—C≡C—CH₃, or any of the alkyl chains of the nitro carboxylic acids mentioned herein. The term “alkyl chain of the nitro carboxylic acid” refers to the nitro carboxylic acid without the carboxylic acid group. As an example the alkyl chain of 9-nitro-cis-hexadecenoic acid is 8-nitro-cis-pentadecen-1-yl.

In other words, the moiety O—R* represents —OH, polyethylene glycolyl, polypropylene glycolyl, cholesteroyl, phytosteroyl, ergosteroyl, coenzyme A or an alkoxy group consisting of 1 to 10 carbon atoms, wherein this alkoxy group may contain one or more double and/or one or more triple bonds and/or may be substituted by one or more nitro groups and/or one or more substituents S¹-S²⁰. Preferably O—R* refers to methanoyl, ethanoyl, propanoyl, iso-propanoyl, butanoyl, sec-butanoyl, iso-butanoyl, tert-butanoyl, vinyl alcoholyl (—O—CH═CH₂), allyl alcoholyl (—O—CH₂—CH═CH₂). Most preferred O—R* represents —OH.

Moreover, as indicated in general formula (X) at least one nitro (—NO₂) group is attached to one of the carbon atoms of the carbon chain. The nitro group shown in general formula (X) does not have a specific position, it can be attached to any of the carbon atoms (α to ω) of the alkyl chain, i.e. the carbon atom chain. Most preferably, the nitro groups or the nitro groups is/are attached to a vinyl moiety of the unsaturated alkyl chain of an unsaturated carboxylic acid, wherein the term unsaturated carboxylic acid also covers unsaturated carboxylic acid esters as defined above. That means that the nitro group(s) is/are most preferably attached to a double bond in the unsaturated alkyl chain of the unsaturated carboxylic acid. However it is possible that the carbon atom chain which can be referred to as alkyl chain may contain more than one nitro group. Moreover the carbon atom chain may also contain double bonds and/or triple bonds and can be linear or branched and can comprise further substituents defined as substituents S¹ to S². Thus the term “alkyl chain” does not only refer to linear and saturated alkyl groups but also refers to mono-unsaturated, poly-unsaturated, branched and further substituted alkyl groups or alkenyl groups or alkynyl groups respectively. The mono-, di- and poly-unsaturated carbon atom chains of the unsaturated carboxylic acids (including unsaturated carboxylic acid esters) are preferred. Most preferred are double bonds in the carbon atom chain of the carboxylic acid while triple bonds and saturated carbon atom chains of the unsaturated carboxylic acid are less preferred.

Thus, the carbon atom chain refers to an alkyl chain to which at least one nitro group is attached consisting of 1 to 40 carbon atoms, wherein this alkyl chain may contain one or more double and/or one or more triple bonds and may be cyclic and/or may be substituted by one or more nitro groups and/or one or more substituents S¹-S²⁰. In case the term “alkyl” is regarded unclear, due to the fact that an alkyl group is saturated and may not contain double or triple bonds, the following definition is provided to replace this section in claim 1 and claim 8: the term

carbon atom chain refers to an alkyl chain or alkenyl chain or alkynyl chain to which at least one nitro group is attached consisting of 1 to 40 carbon atoms, wherein this alkyl chain may be cyclic and may be substituted by one or more nitro groups and/or one or more substituents S¹-S²⁰, the alkenyl chain contains one or more double bonds and may be cyclic and may be substituted by one or more nitro groups and/or one or more substituents S¹-S²⁰, and the alkynyl chain contains one or more triple bonds and may be cyclic and may be substituted by one or more nitro groups and/or one or more substituents S¹-S²⁰. The term “may be substituted by one or more nitro groups” has to be understood in a way that one or more nitro groups may be present on the carbon atom chain in addition to the one nitro group which is necessarily required and explicitly mentioned and drawn in general formula (X).

The term “carbon atom chain” refers to an alkyl chain which is saturated or which may contain one or more double bonds and/or triple bonds or refers to an alkyl chain (only saturated carbon atom chains are meant), alkenyl chain or alkynyl chain to which at least one nitro group is attached which is the nitro group explicitly drawn and mentioned in general formula (X). The carbon atom chain contains preferably 1 to 10, more preferably 1 to 5 double bonds or vinyl moieties. The carbon atom chain consists of 1 to 40 carbon atoms, preferably 2 to 30 carbon atoms and more preferably 4 to 24 carbon atoms, wherein this alkyl chain may contain one or more double and/or one or more triple bonds and/or may be substituted by one or more nitro groups and/or one or more substituents S¹-S²⁰, S¹-S²⁰ represent independently of each other —OH, —OP(O)(OH)₂, —P(O)(OH)₂, —P(O)(OCH₃)₂, —OCH₃, —OC₂H₅, —OC₃H₇, —O-cyclo-C₃H₅, —OCH(CH₃)₂, —OC(CH₃)₃, —OC₄H₉, —OPh, —OCH₂-Ph, —OCPh₃, —SH, —SCH₃, —SC₂H₅, —F, —Cl, —Br, —I, —CN, —OCN, —NCO, —SCN, —NCS, —CHO, —COCH₃, —COC₂H₅, —COC₃H₇, —CO-cyclo-C₃H₅, —COCH(CH₃)₂, —COC(CH₃)₃, —COOH, —COOCH₃, —COOC₂H₅, —COOC₃H₇, —COO-cyclo-C₃H₅, —COOCH(CH₃)₂, —COOC(CH₃)₃, —OOC—CH₃, —OOC—C₂H₅, —OOC—C₃H₇, —OOC-cyclo-C₃H₅, —OOC—CH(CH₃)₂, —OOC—C(CH₃)₃, —CONH₂, —CONHCH₃, —CONHC₂H₅, —CONHC₃H₇, —CON(CH₃)₂, —CON(C₂H₅)₂, —CON(C₃H₇)₂, —NH₂, —NHCH₃, —NHC₂H₅, —NHC₃H₇, —NH-cyclo-C₃H₅, —NHCH(CH₃)₂, —NHC(CH₃)₃, —N(CH₃)₂, —N(C₂H₅)₂, —N(C₃H₇)₂, —N(cyclo-C₃H₅)₂, —N[CH(CH₃)₂]₂, —N[C(CH₃)₃]₂, —SOCH₃, —SOC₂H₅, —SOC₃H₇, —SO₂CH₃, —SO₂C₂H₅, —SO₂C₃H₇, —SO₃H, —SO₃CH₃, —SO₃C₂H₅, —SO₃C₃H₇, —OCF₃, —OC₂F₅, —O—COOCH₃, —O—COOC₂H₅, —O—COOC₃H₇, —O—COO-cyclo-C₃H₅, —O—COOCH(CH₃)₂, —O—COOC(CH₃)₃, —NH—CO—NH₂, —NH—CO—NHCH₃, —NH—CO—NHC₂H₅, —NH—CO—N(CH₃)₂, —NH—CO—N(C₂H₅)₂, —O—CO—NH₂, —O—CO—NHCH₃, —O—CO—NHC₂H₅, —O—CO—NHC₃H₇, —O—CO—N(CH₃)₂, —O—CO—N(C₂H₅)₂, —O—CO—OCH₃, —O—CO—OC₂H₅, —O—CO—OC₃H₇, —O—CO—O-cyclo-C₃H₅, —O—CO—OCH(CH₃)₂, —O—CO—OC(CH₃)₃, —CH₂F, —CHF₂, —CF₃, —CH₂Cl, —CH₂Br, —CH₂₁, —CH₂—CH₂F, —CH₂—CHF₂, —CH₂—CF₃, —CH₂—CH₂Cl, —CH₂—CH₂Br, —CH₂—CH₂I, —CH₃, —C₂H₅, —C₃H₇, -cyclo-C₃H₅, —CH(CH₃)₂, —C(CH₃)₃, —C₄H₉, —CH₂—CH(CH₃)₂, —CH(CH₃)—C₂H₅, —C₅H₁₁, -Ph, —CH₂-Ph, —CPh₃, —CH═CH₂, —CH₂—CH═CH₂, —C(CH₃)═CH₂, —CH═CH—CH₃, —C₂H₄—CH═CH₂, —CH═C(CH₃)₂, —C≡CH, —C≡C—CH₃, —CH₂—C≡CH, —P(O)(OC₂H₅)₂, cholesteryl (C₂₇H₄₅O—), phosphatidylinositol, nucleotides, ether analogues, lipoamines, dihydrolipoamines, lysobiphospatidic acid, anandamide, long chain N-acyl-ethanolamide, sn-1 substituents with glycerol or diglycerol, sn-2 substituents with glycerol or diglycerol, sn-3 substituents, ceramide, sphingosine, ganglioside, galactosylceramide, aminoethylphosphonic acid.

However, unsaturated nitrocarboxylic acids are preferred and moreover unsaturated nitrocarboxylic acids with one or two nitro groups are preferred.

In the following specific description areas of use are presented in detail. The areas of indication as well as the described types of indications or application of nitrocarboxylic acids and/or their derivatives do not exclude the use in substantially similar indications or states, in which a modification of the healing process or pattern or other use forms are desirable. The inventive nitrocarboxylic acids can be used for the prophylaxis and treatment of all diseases and/or states which display an aggressive healing response or are liable to do so. These diseases and/or states comprise the following groups:

### 1. Medical Device Coating

Another aspect is the response of tissues in persistent contact with foreign materials. Even small deviations in biocompatibility, mostly by chemical substances, lead to a cellular response. Also herein the induction of a healing pattern is dependent on the intensity of the irritation. This often results in the formation of a dense fibrotic wall around the foreign body. Hereby, functional or cosmetic disturbances can result. With the inventive substances the tissue response to a damaging irritation shall be influenced, too. Thus it is possible to reduce this tissue response to contact with a foreign body.

Surprisingly, this problem can be solved by the application of nitrocarboxylic acids or their pharmaceutically acceptable salts or the coating of medical devices that are brought into intimate temporal or permanent contact with tissues/organs with at least one of these compounds. The previously described effects, preferentially causing an active healing pattern of cells in response to the interventional treatment, may be causal for this beneficial effect. Furthermore, the healing of the wound is accelerated by an immediate initiation of the healing phase.

This application is particularly directed to the use of a nitrocarboxylic acid as a surface coating for prophylaxis of a pathophysiological or non-physiological reaction to an irritation which results from medical treatment associated with irritation due to the native implant surface. Coating is applicable for all implants and implant materials irrespective of their form or structure. Materials to be coated include but are not restricted to metals or metal alloys, polymers, tissues (homo-, -allo, -xenografts). The coating includes also instruments (forceps, retractors) and materials (suture material, tubings, and catheters) that are used during medical or cosmetical procedures.

### Medical Implants and Devices

Thus another aspect of the present invention is directed to medical device and medical implants coated with at least one nitrocarboxylic acid of the general formula (X)

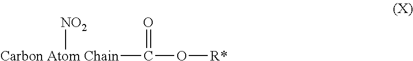

wherein the residues O—R* and “carbon atom chain” are defined as stated above.

According to the invention the terms “medical device” or “medical devices” shall be used as generic term which includes implants of any kind.

A preferred embodiment is the use of coated instruments/material/wound dressings/implants during surgical, plastic or cosmetic procedures causing injuries, wherein said irritation or injury is selected from cut, tear, dissection, resection, suture, wound closure, debridgement, cauterization, suction, drainage, implantation, grafting or fracture. It can also result from an interventional procedure. Implants to be coated with are selected from the group comprising or consisting of tissue replacement implants, breast implants, soft implants, joint implants, cartilage implants, natural or artificial (i.e. Dacron) tissue implants and grafts, autogenous tissue implants, intraocular lenses, surgical adhesion barriers, nerve regeneration conduits, birth control devices, shunts, tissue scaffolds; tissue-related materials including small intestinal submucosal (SIS) matrices, dental devices and dental implants, drug infusion tubes, cuffs, drainage devices (ocular, pulmonary, abdominal, urinary, thecally), tubes (endotracheal, tracheostomy), surgical meshes, ligatures, sutures, staples, patches, slings, foams, pellicles, films, implantable electrical stimulators, pumps, ports, reservoirs, catheters for injection or stimulation or sensing, wound coatings, suture material, surgical instruments such as scalpels, lancets, scissors, forceps or hooks, clinical gloves, injection needles, endoprotheses and exoprotheses.

Osteosynthetic materials (materials suitable for osteosynthesis), catheters (i.e. demers, braunules (=infusion cannulae)), wound dressings such like gels, pates, colloids, glues, alginates, foams, adsorbers, gauze, cotton wool, lint, gamgee, bandages. Suture materials such like sutures, filaments, clips, wires and the like, wound meshes

The inventive nitrocarboxylic acids can also be used for the coating of any other clinically used material that is liable to come into contact with endangered tissues or cells. Examples for such materials are wound coatings, suture material, surgical instruments such as scalpels, lancets, scissors, forceps or hooks, medical devices, clinical gloves, injection needles, endoprotheses, respectively implants, exoprotheses etc. The inventive compounds exert their beneficial and/or protective action via the same mechanisms as described before.

According to the invention arterial implants shall not be covered by the term “implant”. They are expressly disclaimed.

The inventive general principle of nitrated fatty acids to reduce or inhibit a non-physiologic reaction of cell which have come in contact with nitrated fatty acids to an irritant which have been proved in clinically relevant setting as shown in the examples, assures their broad use in a variety of medical or cosmetical procedures utilizing various devices and implants which are brought in intimate contact with body tissue. The above mentioned procedures and devices or implants can be used in a broad spectrum of clinical settings that comprises cosmetic, esthetic or therapeutic measures that have an inherent risk of an adverse reaction of the affected cells, tissues or organs. In a preferred embodiment, clinical conditions or diseases are: burnings, celoids, hernia repair, nerve traumatization, necrosis debridgement, breast reconstruction using an implant. These are examples of indications in which the demonstrated effects of nitrated fatty acids inhibit the pathohphysiological stimulation which causes a high rate of pathologic healing pattern in those indications.

### 2. Protection and Therapy of an Aggressive Healing Pattern as Response to Alteration, Damage or Traumatisation of Tissues Due to Surgical or Interventional Manipulation or Injuries

The inventive nitrocarboxylic acids are also useful for preventing, reducing or treating a pathophysiological or non-physiological healing process or an inappropriate or undesirable tissue formation or fusion. One aspect of organ protection is the prophylaxis or treatment of a tissue or organ response to endogeneous or exogeneous damage. These types of damage can be physical (a.o. mechanical, thermal), chemical (a.o. metabolic), or electrical. This damage can be in the form of a mechanical wound, an injury, a cut, dissections, resections, debridgments, a contusion, a burn, burning frostbites, aphthous ulcers, granuloma, necrosis, cauterization (chemical burn), a fracture, suction, strains, surgical drains, implantations etc. The severity of the cell damage is decisive whether the reaction to the irritation induces an active or an aggressive healing stimulus. Surprisingly, a reduction or even an inhibition of the initiation of an aggressive healing pattern could be shown herein by systemic or local application of nitrocarboxylic acids or their derivatives.

A further aspect of tissue protection concerns medical interventions for supporting or inducing wound closure or wound healing, for example as a consequence of a trauma. Surgical procedures are typically accompanied by the damage of healthy tissue. Tissues are often separated from each other, surgically removed or sewn. Wound surfaces with damaged tissue result. This may lead to an aggressive healing process, too. Often a massive aggregation of connective tissue layers occurs. Stiffness of the affected tissue layers results which may entail functional and/or cosmetic defects. Finding an access through such scarred tissue is much more difficult; in some cases a necessary operation may even not be performed. By initiating an active healing process scarring of this type can be avoided to a large extent.

The present application is also directed to the use of a nitrocarboxylic acid for the treatment or prophylaxis of a pathophysiological or non-physiological reaction to an irritation which results from medical treatment associated with potential irritation or injury of cells, organs or tissues, or from surgical, plastic or cosmetic procedures causing injuries, wherein said irritation or injury is selected from cut, tear, dissection, resection, suture, wound closure, debridgement, cauterization, suction, drainage, implantation, grafting, fracture or osteosynthesis. It can also result from an interventional procedure, such as aspiration, radiation or laser or tissue welding.

The nitrocarboxylic acids can be applied systemically, locally or via a medical device (see below).

Preferred clinical situations/diseases in which nitrated fatty acids excerts beneficial effects are but are restricted to nerve destructions, tumors of the ZNS, keloids, cataract, tissue augmentation, laser ablation, burns or treatment of any trauma, any type of surgery or tissue suturing or adaptation.

Thus the present application is directed to the use of a nitrocarboxylic acid for inhibiting cells, organells or tissues to develop a pathophysiological or non-physiological reaction to an irritation.

Surprisingly, this problem can be solved by the application of nitrocarbmlic acids or their pharmaceutically acceptable salts or the coating of medical devices. The previously described effects, preferentially causing an active healing pattern of cells in response to the interventional treatment, have been proven to be causal for this beneficial effect. Furthermore, the healing of the wound is accelerated by an immediate initiation of the healing phase.

3. Protection of Tissues, In Situ or Ex Vivo Organs, or Transplants from Cold Preservation Impairment

The interventional or surgical treatment of tissues or organs often requires a temporary interruption of the blood flow. To protect the tissue/organ from damage several methods to preserve organs form damage due to energy supply. Hypothermia is a commonly used for this purpose, with lower tissue temperatures allowing longer periods of tissue or organ protection. However, lower temperatures can cause damage to the cell membrane and induce necrosis (Apoptosis versus necrosis during cold storage and rewarming of human renal proximal tubular cells. Salahudeen A K, Joshi M. Jenkins J K. Transplantation. 2001 Sep. 15; 72(5):798-804). Cold preservation induced injury has been found to have a different mechanisms of injury by direct alteration of the membrane components and of the cytosceleton. Substances that are known to partitionate in the cell membrane were found to reduce cold preservation induced injury. (Improved cold preservation of kidney tubular cells by means of adding bioflavonoids to organ preservation solutions. Ahlenstiel T. Burkhardt G, Köhler H, Kuhlmann M K., Transplantation. 2006 Jan. 27; 81(2):231-9).

Nitrated fatty acids (also named nitrocarboxylic acids herein) have been found to have membrane stabilisating effects as could be shown in the examples. Surprisingly, the physico-chemical changes induced due to the partition of nitrated fatty acids within a cell membrane were found to enhance resistance of the cell membrane against cold induced changes.

Surprisingly, the reaction of cells, respectively the tissue to such damages can be delayed or even completely inhibited by the prior or subsequent, local and/or systemic application of nitrocarboxylic acids. The exposure time and the time frame during which the application should be performed can vary considerably between the cell and tissue types, corresponding to the extent of the damage. This also holds true for the dosage and the pharmaceutical formulation of nitrocarboxylic acids and their derivatives.

Thus the inventive nitrocarboxylic acid compounds can be used for cold preservation of tissues and organs in the pre-, inter- and post-operative phase, and applied to tissues to be protected for organ protection and in organ transplants. Preferred indications are but are not restricted to graft transplantation, free tissue transplantation for defect filling i.e. after tumor or necrosis resection, organ or tissue plastic i.e. formation of a pouch, tissue or organ donation.

### 4. Stabilization of Membrane Functions in Cells and Organelles

Membranes in cells and organelles have many distinct functions. To name a few of them, some cardiac cells depolarize at regular time intervals thus providing a regular heart beat. Others have to transmit electrical impulses, while others sense physical or chemical stimuli. These membrane functions are generally provided by specialized structures and a particular composition of membrane components. Herein membrane proteins play a key role. They are integrated into the phospholipid layer of the membrane. Recent findings show that the function of membrane proteins can be influenced by the surrounding phospholipids. In a clinical study it could be shown that the rate of sudden death in persons with an increased risk of heart failure could be reduced by the regular prophylactic administration of fatty acids. Surprisingly, by applying nitrocarboxylic acids several cell functions including electrical stability is maintained and stabilized against internal and external influences.

Examples of diseases that can be thus treated with nitrocarboxylic acids include, but are not limited to cardiac rhythm disturbances (cardiac arrhythmias) such as atrial extrasystoles, atrial flutter, atrial fibrillation, ventricular extrasystoles, ventricular tachycardia, torsades de pointes, ventricular flutter, ventricular fibrillation, Wolff-Parkinson-White syndrome, Lown-Ganong-Levine syndrome, as well as acute or chronic pain, hypersensitivity syndrome, neuropathic pain, atopies such as urticaria, allergic rhinitis and hay fever, enteropathies such as tropical sprue or coeliac disease.

Thus this invention also refers to a use of a nitrocarboxylic acid according for the prophylaxis and treatment of a pathophysiological or non-physiological reaction of cell membranes which affects the properties, function and reactivity of cell, organelle or plasma membranes and results from chronic or acute irritation or stimulation. This chronic or acute irritation or stimulation can be caused by a physical trauma, chemical trauma, electrical trauma, poisons or toxins, immunological biomolecules and malnutrition.

### 5. Special Situations of Endo- and Exogenous Cell or Tissue Damage

Also diseases including pathophysiological or non-physiological fibroblast proliferation may be treated with the inventive compounds. They can also be used for their prophylaxis.

Thus this application is also directed to the use of a nitrocarboxylic acid for the treatment or prophylaxis of a pathophysiological or non-physiological reaction to an irritation which results from an exogenous irritation, wounding or trauma, such as burn, chemical burn, alkali burn, burning, hypothermia, frostbite, cauterization, granuloma, necrosis, ulcer, fracture, foreign body reaction, cut, scratch, laceration, bruise, tear, contusion, fissuring or burst. Alternatively, the pathophysiological or non-physiological reaction to an irritation can result from an endogenous irritation or stimulation by acute or chronical physical, chemical or electrical means. A typical example of a chronic mechanical irritation is fasciculitis and epicondylitis or their form of tendonitis, neuropathy or prostate hypertrophy.

### 6. Use in Diseases or States Due to Toxin Accumulation

The inventive nitrocarboxylic acids can also be used for the treatment of diseases and/or states in which a toxin accumulates in an organ or the whole organism. It can also be used for the propylaxis if such a toxin accumulation has to be seriously feared, especially in high-risk subjects.

Toxic effects may also arise from exposure or ingestion of poisons, and organic or inorganic chemicals. Other reasons may stem from chronic or acute irritation or stimulation, physical, chemical or electrical trauma, immunological biomolecules and malnutrition.

The invention thus refers also to the treatment or prophylaxis of diseases and states associated with a toxin or poison, such as neuropathy, acute pain, chronic pain, hypersensitivity syndrome, neuropathic pain, burning feet syndrome, induratio fibroplastica penis and Sudeck's atrophy.

Nitrated fatty acids have shown to reduce or inhibit reactions to the irritating stimulus that include a large variety of irritants as shown in the examples. Therefore topical, local or systemic applications of nitrated fatty acids are useful in but not restricted to forenamed clinical situations/diseases.

In summary, according to the invention nitrocarboxylic acids can be used for inhibiting cells, organells or tissues to develop a pathophysiological or non-physiological reaction to a stimulus which, if not treated, would lead to an aggressive healing response.

7. Use in Diseases and States with an Additional Inflammatory Component

It was set forth in the introductory part that it must be differentiated between genuine inflammations and diseases and/or states with an inflammatory component.

It should be noted that the inventive nitrocarboxylic acids shall not be used for the treatment of genuine inflammations. But they may be used for the treatment and/or prophylaxis of accompanying pathological or non-physiological healing response patterns in diseases or states which may include such an inflammatory component. It is not intended for prophylaxis or therapy of the causative disease with an inflammatory component.

Likewise, there are diseases and states with an immunologic component. They must be differentiated in the same manner from genuine immunologic diseases.

The beneficial effects of the inventive nitrocarboxylic acids refers to cell, organelle or tissue changes that occur before a genuine inflammation or a genuine immunologic disease becomes manifest or affects their structures.

As known in the art, the response to the same irritation of a tissue, cell or organelle can completely diverge within an organism, due to differences in local conditions that usually are beyond predictability. Accordingly, various clinical situations are known to be associated with a risk of an aggressive healing pattern, which could be prevented or treated by nitrocarboxylic acids, therefore their use is indicated in the named clinical conditions. This should not be restricted to the medical indications claimed but can be extended to all clinical situations except genuine inflammations. However, surgical or interventional procedures with an inherent risk of an aggressive healing are not excluded when performed at the presence of a coincident genuine inflammation since the beneficial effects refer to the surgical trauma and not to the genuine inflammation.

Nitrocarboxylic acids are preferentially indicated in diseases which additionally display an acute or chronic primary degenerative course in order to reduce the known reactive changes of the connective tissues, notably fibrosis. Examples for such diseases are osteomyelofibrosis, chronic polyarthritis, atrophia of mucuous tissues or epidermis, dermatitis ulcerosa, connective tissue diseases such dermatomyositis, chronic vasculitis, polyarteritis nodosa, hypersensitivity angiitis, Takayasu's arteritis, Wegener's granulomatosis, Kawasaki disease, Buerger's disease, non-tropical sprue, prostate hypertrophy, arthropathy, peri-arthropathy, fibromyalgia, meralgia paresthetica, carpal tunnel syndrome and nerve compression syndrome.

Thus this invention also refers to the use of a nitrocarboxylic acid for the treatment, diagnosis or prophylaxis of a fibrosis or a pathophysiological or non-physiological reaction to an irritation results from a disease with an inflammatory component which is not a genuine inflammatory disease. Such a disease with an inflammatory component is to be selected from enteropathies such as tropical sprue or coeliac disease, or from bronchiectasis, emphysema, chronic obstructive pulmonary disease (COPD), dermatoses such as atrophic contact dermatosis, or from gouty arthritis, osteoarthrosis, degenerative arthrotic conditions, toxic shock syndrome, amyolidosis, dermatitis ulcerosa and nephrosclerosis. Alternatively, this therapeutic approach refers also to an immunological process or disease which is not a genuine inflammatory disease, such as cystic fibrosis, atopic dermatose, atrophy of mucuous tissue or epidermis, connective tissue diseases such as Sharp syndrome and dermatomyositis, aphthous ulcer. Stevens-Johnson syndrome, or toxic epidermal necrolysis.

### Nitrocarboxylic Acids

Nitrocarboxylic acids are a subgroup of carboxylic acids (organic acids) characterized by at least one nitro group replacing a hydrogen atom. Thus, the nitrocarboxylic acids which are used in accordance with the present invention are carboxylic acid having in total between 2 and 50, preferably between 4 and 40 and more preferably between 6 and 30 carbon atoms (in total including side chains, substituents and the carboxylate carbon atom) while the alkyl chain or carbon atom chain of the nitrocarboxylic acid can be saturated, olefinic, acetylenic, polyunsaturated, linear or branched and may contain further substituent in addition to the at least one nitro group. The one or more further substituents S¹-S²⁰ which might be present on the alkyl chain or carbon atom chain of the nitrocarboxylic acids are selected from the group comprising or consisting of: —OH, —OP(O)(OH)₂, —P(O)(OH)₂, —P(O)(OCH₃)₂, —OCH₃, —OC₂H₅, —OC₃H₇, —O-cyclo-C₃H₅, —OCH(CH₃)₂, —OC(CH₃)₃, —OC₄H₉, —OPh, —OCH₂-Ph, —OCPh₃, —SH, —SCH₃, —SC₂H₅, —F, —Cl, —Br, —I, —CN, —OCN, —NCO, —SCN, —NCS, —CHO, —COCH₃, —COC₂H₅, —COC₃H₇, —CO-cyclo-C₃H₅, —COCH(CH₃)₂, —COC(CH₃)₃, —COOH, —COOCH₃, —COOC₂H₅, —COOC₃H₇, —COO-cyclo-C₃H₅, —COOCH(CH₃)₂, —COOC(CH₃)₃, —OOC—CH₃, —OOC—C₂H₅, —OOC—OC-cyclo-C₃H₅, —OOC—CH(CH₃)₂, —OOC—C(CH₃)₃, —CONH₂, —CONHCH₃, —CONHC₂H₅, —CONHC₃H₇, —CON(CH₃)₂, —CON(C₂H₅)₂, —CON(C₃H₇)₂, —NH₂, —NHCH₃, —NHC₂H₅, —NHC₃H₇, —NH-cyclo-C₃H₅, —NHCH(CH₃)₂, —NHC(CH₃)₃, —N(CH₃)₂, —N(C₂H₅)₂, —N(C₃H₇)₂, —N(cyclo-C₃H₅)₂, —N[CH(CH₃)₂]₂, —N[C(CH₃)₃]₂, —SOCH₃, —SOC₂H₅, —SOC₃H₇, —SO₂CH₃, —SO₂C₂H₅, —SO₂C₃H₇, —SO₃H, —SO₃CH₃, —SO₃C₂H₅, —SO₃C₃H₇, —OCF₃, —OC₂F₅, —O—COOCH₃, —O—COOC₂H₅, —O—COOC₃H₇, —O—COO-cyclo-C₃H₅, —O—COOCH(CH₃)₂, —O—COOC(CH₃)₃, —NH—CO—NH₂, —NH—CO—NHCH₃, —NH—CO—NHC₂H₅, —NH—CO—N(CH₃)₂, —NH—CO—N(C₂H₅)₂, —O—CO—NH₂, —O—CO—NHCH₃, —O—CO—NHC₂H₅, —O—CO—NHC₃H₇, —O—CO—N(CH₃)₂, —O—CO—N(C₂H₅)₂, —O—CO—OCH₃, —O—CO—OC₂H₅, —O—CO—OC₃H₇, —O—CO—O-cyclo-C₃H₅, —O—CO—OCH(CH₃)₂, —O—CO—OC(CH₃)₃, —CH₂F, —CHF₂, —CF₃, —CH₂Cl, —CH₂Br, —CH₂₁, —CH₂—CH₂F, —CH₂—CHF₂, —CH₂—CF₃, —CH₂—CH₂Cl, —CH₂—CH₂Br, —CH₂—CH₂₁, —CH₃, —C₂H₅, —C₃H₇, -cyclo-C₃H₅, —CH(CH₃)₂, —C(CH₃)₃, —C₄H₉, —CH₂—CH(CH₃)₂, —CH(CH₃)—C₂H₅, —C₅H₁₁, -Ph, —CH₂-Ph, —CPh₃, —CH═CH₂, —CH₂—CH═CH₂, —C(CH₃)═CH₂, —CH═CH—CH₃, —C₂H₄—CH═CH₂, —CH═C(CH₃)₂, —C≡CH, —C≡C—CH₃, —CH₂—C≡CH, —P(O)(OC₂H₅)₂, C₂₇H₄₅O-(cholesteryl), nucleotides, lipoamines, dihydrolipoamines, lysobiphospatidic acid, anandamide, long chain N-acyl-ethanolamide, sn-1 substituents with glycerol or diglycerol, sn-2 substituents with glycerol or diglycerol, on-3 substituents, ceramide, sphingosine, galactosylceramide, aminoethylphosphonic acid.

According to the invention the aforementioned nitrocarboxylic acids shall be used for the prophylaxis or therapy of the medical conditions or diseases listed in the following chapters.

Moreover, the nitrocarboxylic acids used within the present invention have at least one nitro group (—NO₂) which can be attached to any one of the carbon chain atoms including any side chains.

A preferred subgroup of nitrocarboxylic acids are nitro-fatty acids. Fatty acids have in general a long aliphatic chain which can be unsaturated or which can comprise one or more double bonds and/or one or more triple bonds.

Examples for nitrocarboxylic acids with saturated alkyl chains are: nitrooctanoic acid (nitrocaprylic acid), nitrodecanoic acid (nitrocaprinic acid), nitrododecanoic acid (nitrolauric acid), nitrotetradecanoic acid (nitromyristic acid), nitrohexadecaoic acid (nitropalmitic acid), nitroheptadecanoic acid (nitromargaric acid), nitrooctadecanoic acid (nitrostearic acid), nitroeicosanoic acid (nitroarachidic acid), nitrodocosanoic acid (nitrobehenic acid), nitrotetracosanoic acid (nitrolignoceric acid). These and other saturated nitrocarboxylic acids may contain 1, 2, 3, 4, 5 or 6 further nitro groups and may contain one or more of the substituents S¹-S²⁰ as mentioned above.

According to the invention a preferred subgroup of nitrocarboxylic acids are unsaturated nitrocarboxylic acids. According to the invention cis and trans isomers as well as (depending on the substituents which can generate chiral centers) enantiomers, diastereomers and their racemates can be used. The nitro group can be bound to any feasible position of the carbon chain.

Preferred unsaturated nitrocarboxylic acids are: nitro-cis-9-tetradecenoic acid (nitromyristoleic acid), nitro-cis-9-hexadecenoic acid (nitropalmitoleic acid), nitro-cis-6-hexadecenoic acid (nitrosalpenic acid), nitro-cis-6-octadecenoic acid (nitropetroselinic acid), nitro-cis-9-octadecenoic acid (nitrooleic acid), nitro-cis-11-octadecenoic acid (nitrovaccenic acid), nitro-cis-9-eicosenoic acid (nitrogadoleinic acid), nitro-cis-11-eicosenoic acid (nitrogondoic acid), nitro-cis-13-docosenoic acid (nitroerucic acid), nitro-cis-15-tetracosenoic acid (nitronervonic acid), nitro-t9-octadecenoic acid (nitroelaidic acid), nitro-t11-octadecenoic acid (nitro-t-vaccenic acid), nitro-t3-hexadecenoic acid, nitro-9,12-octadecadienoic acid (nitrolinoleic acid), nitro-6,9,12-octadecatrienoic acid (nitro-γ-linoleic acid), nitro-8,11,14-eicosatrienoic acid (nitrodihomo-γ-linoleic acid), nitro-5,8,11,14-eicosatrienoic acid (nitroarachidonic acid), nitro-7,10,13,16-docosatetraenoic acid, nitro-4,7,10,13,16-docosapentaenoic acid, nitro-9,12,15-octadecatrienoic acid (nitro-α-linolenic acid), nitro-6,9,12,15-octadecatetraenic acid (nitrostearidonic acid), nitro-8,11,14,17-eicosatetraenoic acid, nitro-5,8,11,14,17-eicosapentaenoic acid (nitro-EPA), nitro-7,10,13,16,19-docosapentaenoic acid (nitro-DPA), nitro-4,7,10,13,16,19-docosahexaenic acid (nitro-DHA), nitro-5,8,11-eicosatrienoic acid (nitromead acid), nitro-9c 11t 13t eleostearinoic acid, nitro-8t 10t 12c calendic acid, nitro-9c 11t 13c catalpic acid, nitro-4,7,9,11, 13, 16, 19 docosaheptadecanoic acid (nitrostellaheptaenoic acid), nitrotaxolic acid, nitropinolenic acid, nitrosciadonic acid, nitro-6-octadecinoic acid (nitrotariric acid), nitro-t11-octadecen-9-ynoic acid (nitrosantalbic or nitroximenic acid), nitro-9-octadecynoic acid (nitrostearolic acid), nitro-6-octadecen-9-ynoic acid (nitro-6,9-octadecenynoic acid), nitro-t10-heptadecen-8-ynoic acid (nitropyrulic acid), nitro-9-octadecen-12-ynoic acid (nitrocrepenic acid), nitro-t7,t11-octadecadiene-9-ynoic acid (nitroheisteric acid), nitro-t8,t10-octadecadiene-12-ynoic acid and nitro-5,8,11,14-eicosatetraynoic acid (nitro-ETYA).

Particularly preferred are 12-nitro-linoleic acid, 9-nitro cis-oleic acid, 10-nitro-cis-linoleic acid, 10-nitro-cis-oleic acid, 5-nitro-eicosatrienoic acid, 16-nitro-all-cis-4,7,10,13,16-docosapentaenoic acid (nitro-Osbond acid), 9-nitro-all-cis-9-12,15-octadecatrienoic acid (nitro-linolenic acid), 14-nitro-all-cis-7,10,13,16,19-docosapentaenoic acid (nitro-EPA), 15-nitro-cis-15-tetracosenoic acid (nitro-nervonic acid), 9-nitro-trans-oleic acid, 9,10-nitro-cis-oleic acid, 13-nitro-octadeca-9,11,13-trienoic acid (nitro-punicic acid), 10-nitro-trans-oleic acid, 9-nitro-cis-hexadecenoic acid, 11-nitro-5,8,11,14-eicosatrienoic acid, 9,10-nitro-trans-oleic acid, 9-nitro-9-trans-hexadecenoic acid (nitro-palmitoleic acid), 13-nitro-cis-13-docosenoic acid (nitro-erucic acid), 8,14-nitro-cis-5,8,11,14-eicosatetraenoic acid (dinitro-arachidonic acid), 4,16-nitro-docosahexaenoic acid (nitro-DHA), 9-nitro-cis-6,9,12-octadecatrienoic acid (nitro-GLA), 6-nitro-cis-6-octadecenoic acid (nitro-petroselinic acid) and 11-nitro-cis-5,8,11,14-eicosatetraenoic acid (nitro-arachidonic acid).

Preferred embodiments are nitro-oleic acids such as nitro-ETYA, nitro-linoleic acids, nitro-arachidonic acids, 10-nitro-linoleic acid, 12-nitro-linoleic acid, 9-nitro-oleic acid and 10-nitro-oleic acid. In case the position of the nitro group is not indicated or further defined such as 9-nitro-oleic, it is referred to a mixture of nitrocarbondic acids such as a mixture of nitro-oleic acids and especially it is referred to such a mixture of nitrocarboxylic acids as obtained according to the reaction procedure to prepare these nitrocarboxylic acids.

Another embodiment is the use of dinitrocarboxylic acids. The position of the two nitro groups is freely eligible. Particularly preferred is nitro-ETYA.

A preferred subgroup of the nitrocarboxylic acids which can be used according to the present invention, have at least one double bond and have at least one nitro group which is preferably attached to a carbon atom of the olefin moiety as shown in general formula (I), i.e. a carbon atom of the double bond or in alpha position to a double bond as shown in general formula (II). The preferred nitrocarboxylic acids are represented by the following general formula (I) or (II):

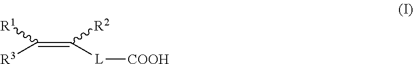

wherein
at least one of R¹ and R² is a nitro (—NO₂) group and the other substituent of R¹ and R² is a nitro group, hydrogen or an alkyl residue comprising 1 to 5 carbon atoms;
R³ is hydrogen or an alkyl chain of 1 to 20 carbon atoms, wherein this alkyl chain can be substituted by one or more of the substituents S¹-S²⁰ and can also be substituted by one or more nitro groups (—NO₂) and/or can contain further double and/or triple bonds;
L represents an alkyl linker of 1 to 20 carbon atoms, wherein this alkyl linker can be substituted by one or more of the substituents S¹-S²⁰ and optionally by one or more nitro groups (—NO₂) and/or can contain further double and/or triple bonds, the following general formula (II):

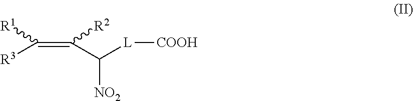

wherein
R¹ and R² are independently of each other selected from a nitro group, hydrogen or an alkyl residue comprising 1 to 5 carbon atoms;
R³ is hydrogen or an alkyl chain of 1 to 20 carbon atoms, wherein this alkyl chain can be substituted by one or more of the substituents S¹-S²⁰ and can also be substituted by one or more nitro groups (—NO₂) and/or can contain further double and/or triple bonds;
L represents in general formula (I) and (II) an alkyl linker of 1 to 20 carbon atoms, wherein this alkyl linker can be substituted by one or more of the substituents S¹-S²⁰ and optionally by one or more nitro groups (—NO₂) and/or can contain further double and/or triple bonds and in case R¹ and/or R² represent an alkyl residue comprising 1 to 5 carbon atoms, this alkyl residue can be substituted by one or more of the substituents S¹-S²⁰ and optionally by one or more nitro groups (—NO₂) and/or can contain further double and/or triple bonds.

Most preferably the nitrocarboxylic acids or nitrocarboxylic acid esters derived from the following fatty acids by nitration (introduction of at least one nitro group) and subsequent esterification if desired or by first esterification and thereafter nitration: hexanoic acid, octanoic acid, decanoic acid, dodecanoic acid, tetradecanoic acid, hexadecanoic acid, heptadecanoic acid, octadecanoic acid, eicosanoic acid, docosanoic acid, tetracosanoic acid, cis-9-tetradecenoic acid, cis-9-hexadecenoic acid, cis-6-octadecenoic acid, cis-9-octadecenoic acid, cis-11-octadecenoic acid, cis-9-eicosenoic acid, cis-11-eicosenoic acid, cis-13-docosenoic acid, cis-15-tetracosenoic acid, t9-octadecenoic acid, t11-octadecenoic acid, t3-hexadecenoic acid, 9,12-octadecadienoic acid, 6,9,12-octadecatrienoic acid, 8,11,14-eicosatrienoic acid, 5,8,11,14-eicosatetraenoic acid, 7,10,13,16-docosatetraenoic acid, 4,7,10,13,16-docosapentaenoic acid, 9,12,15-octadecatrienoic acid, 6,9,12,15-octadecatetraenoic acid, 8,11,14,17-eicosatetraenoic acid, 5,8,11,14,17-eicosapentaenoic acid, 7,10,13,16,19-docosapentaenoic acid, 4,7,10,13,16,19-docosahexaenoic acid, 5,8,11-eicosatrienoic acid, 9c 11t 13t eleostearic acid, 8t 10t 12c calendic acid, 9c 11t 13c catalpic acid, 4, 7, 9, 11, 13, 16, 19 docosaheptadecanoic acid, taxoleic acid, pinolenic acid, sciadonic acid, 6-octadecynoic acid, t11-octadecen-9-ynoic acid, 9-octadecynoic acid, 6-octadecen-9-ynoic acid, t10-heptadecen-8-ynoic acid, 9-octadecen-12-ynoic acid, t7,t11-octadecadiene-9-ynoic acid, t8,t10-octadecadiene-12-ynoic acid, 5,8,11,14-eicosatetraynoic acid, retinoic acid, isopalmitic acid, pristanic acid, phytanic acid, 11,12-methyleneoctadecanoic acid, 9,10-methylenhexadecanoic acid, coronaric acid, (R,S)-lipoic acid, (S)-lipoic acid, (R)-lipoic acid, 6,8-bis(methylsulfanyl)-octanoic acid, 4,6-bis(methylsulfanyl)-hexanoic acid, 2,4-bis(methylsulfanyl)-butanoic acid, 1,2-dithiolane carboxylic acid, (R,S)-6,8-dithiane octanoic acid, (R)-6,8-dithiane octanoic acid, (S)-6,8-dithiane octanoic acid, tariric acid, santalbic acid, stearolic acid, 6,9-octadecenynoic acid, pyrulic acid, crepenynic acid, heisteric acid, t8,t10-octadecadiene-12-inoic acid. ETYA, cerebronic acid, hydroxynervonic acid, ricinoleic acid, lesquerolic acid, brassylic acid and thapsic acid.

Examples of nitrocarboxylic acids falling under general formula (I) or (II) are:

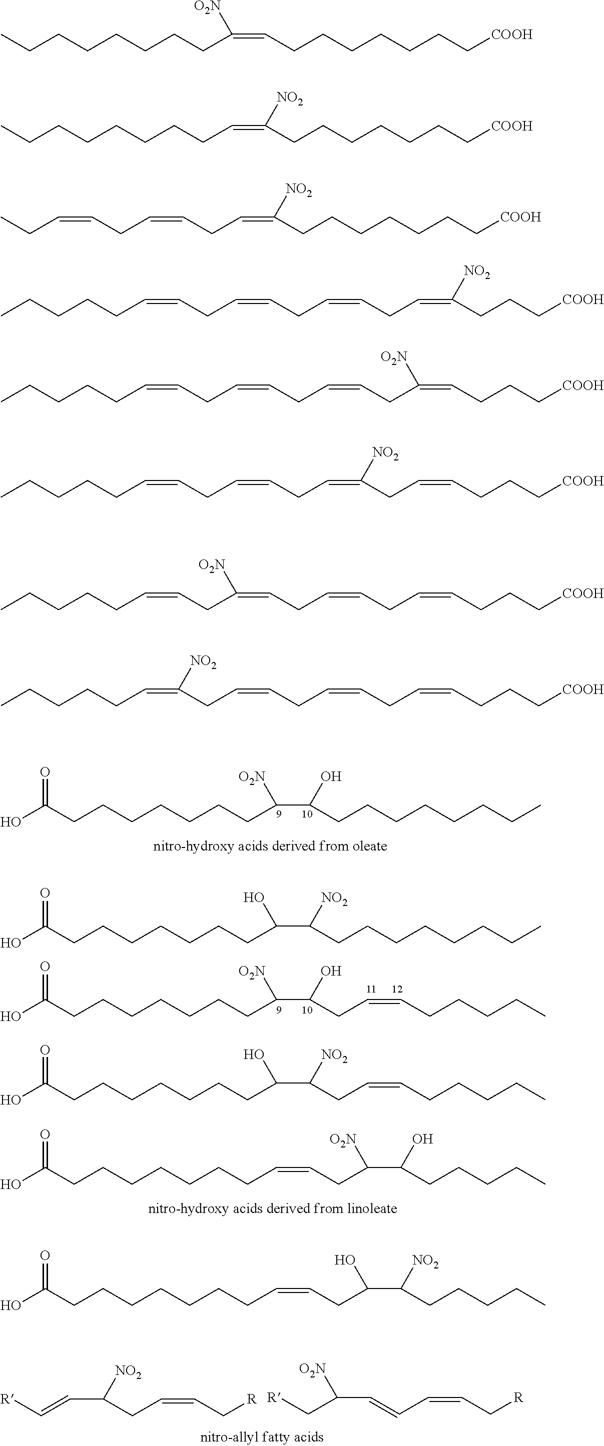

Methods for synthesizing nitrocarboxylic acids are disclosed in Gorczynski, Michael J.; Huang, Jinming; King, S. Bruce; Organic Letters, 2006, 8, 11, 2305 2308 and are shown in FIGS. 1 to 5.

In another preferred embodiment of the present invention the nitrocarboxylic acids are esterified. That means the carboxylic acid group is converted to an ester using an alcohol. Suitable alcohols which can be used to prepare the nitrocarboxylic acid esters are methanol, ethanol, propanol, iso-propanol, butanol, sec-butanol, iso-butanol, tert-butanol, vinyl alcohol, allyl alcohol, polyethylene glycol, polypropylene glycol, cholesterol, phytosterol, ergosterol, coenzyme A or any other alcohol having an carbon atom chain of 1 to 10 carbon atoms wherein this carbon atom chain may contain one or more double and/or one or more triple bonds and/or may be substituted by one or more nitro groups and/or one or more substituents S¹-S²⁰.

It has to be mentioned that according to the invention it is not necessary to use pure nitrocarboxylic acids. Mixtures of various nitrocarboxylic acids can be used within the present invention which could be obtained from one carboxylic acid as well as from different carboxylic acids.

According to the invention all pharmaceutically acceptable salts of the aforementioned nitrocarboxylic acids can be used. Nitrocarboxylic acids can build salts by dissociating a H⁺ from the carboxylic acid group, building an organic or inorganic base.

Examples for suitable organic and inorganic bases are bases derived from metal ions, e.g., aluminum, alkali metal ions, such as sodium of potassium, alkaline earth metal ions such as calcium or magnesium, or an amine salt ion or alkali- or alkaline-earth hydroxides, -carbonates or -bicarbonates. Examples include aqueous sodium hydroxide, lithium hydroxyed, potassium carbonate, ammonia and sodium bicarbonate, ammonium salts, primary, secondary and tertiary amines, such as, e.g., lower alkylamines such as methylamine, t-butylamine, procaine, ethanolamine, arylalkylamines such as dibenzylamine and N,N-dibenzylethylenediamine, lower alkylpiperidines such as N-ethylpiperidine, cycloalkylamines such as cyclohexylamine or dicyclohexylamine, morpholine, glucamine. N-methyl- and N,N-dimethylglucamine, 1-adamantylamine, benzathine, or salts derived from amino acids like arginine, lysine, ornithine or amides of originally neutral or acidic amino acids or the like.

Cells sense a variety of physical and chemical stimuli; however, in most instances, a certain threshold has to be reached or several stimuli (mediators) must come together to act as an irritant and cause a cell reaction. That is the reason why in most nonphysiologic and pathologic conditions several pathways have to be activated or passivated at the same time to induce cell events, like migration, proliferation, apoptosis, or production of matrix proteins. There is so far no substance known that enables complete inhibition of those responses to an irritating stimulus (in clinical conditions/diseases).

However, cell response to irritating stimuli is reduced when cells are preserved by cold. This effect is accomplished by the physical changes of the cell membranes. The denser packed membranes reduce the sensing capacity of receptors and adhesion molecules (Anbazhagan V, Schneider D (2010)). The membrane environment modulates self-association of the human GpA TM domain-implications for membrane protein folding and transmembrane signaling (Biochim Biophys Acta 1798(10):1899-1907). Increasing the hydrophobicity of the cell membranes has a similar effect. Therefore, adjusting cell membrane hydrophobicity accompanied by a change in the density of the phospholipid bilayer could have similar effects to cold preservation. In fact, an increase in hydrophobicity accompanied with a significant change of physical membrane properties was instituted by nitrated fatty acids. As documented in various experiments and outlined in part in the examples, physical and physicochemical properties of cell membranes are altered by simple partitioning of nitrated fatty acids into the cell membrane, thereby reducing cell nociception at the cell membrane level without specifically interfering with receptors of cell signaling molecules. As further set forth in the examples, attenuation of cell nod- and/or perception at the membrane level explains why nitrated fatty acids can be successfully used in various conditions, reducing or inhibiting the response of an irritating stimulus. Since the effect on cell nociception of nitrated fatty acids could be demonstrated in various cell types it can be concluded that this principal of action is transferable comparable cell lines in other clinical conditions as well. Furthermore, experiments proved that (1) nitrated fatty acids reduce or block the nociception/perception of key irritating stimuli that are of physical (shear stress) or chemical (toxins, mediators) origin and that (2) typical responses, playing a key role in various irritation-induced diseases or clinical settings, are diminished or completely absent.

Since the nitro group in general enhances hydrophobicity of a fatty acid molecule, which displays the common principle of the effects documented in the examples, superiority of nitrated fatty acids as compared to native fatty acids in modulating nociception and/or stimulus perception is obvious for other nitrated unsaturated fatty acids as well.

Interference of nitrated fatty acids with cell key mediators has been documented in the scientific literature. Such an interference with PPAR gamma receptor or up-regulation of hemoxygenase expression could possibly contribute to an attenuation of signal transduction in certain pathways resulting in migration, proliferation, and even apoptosis, and therefore may stand against the effects of nitrated fatty acids. However, this is not the case because of several reasons: (1) Neither the blocking or activation of those pathways alone or in combination leading to complete inhibition of migration, proliferation, or production of extracellular matrix has been documented so far, (2) the change in physical/physicochemical membrane properties precedes intracellular pathway interactions or gene expression, (3) cells exposed to nitrated fatty acids have not been activated by an irritant; therefore, a stimulation of hemoxygenase would bear no consequence for these cells, (4) the proved key element of action of nitrated fatty acids, namely the altered nociception and perception of the TRP receptor family, does not share any similarities with pathway interferences that have been documented so far. Furthermore as described in the examples, it could be shown that nitrocarboxylic acids exert their antifibrotic effects independent form PPRA activation or hemoxygenase-I production.

As stated above, various disorders and clinical conditions result in typical sets, compositions and/or sequelae of irritant stimuli that lead to uniform responses in various cell types. Therefore, in clinical conditions or diseases, which cause a nonphysiological or pathological healing pattern due to an irritant of the same kind in one cell population, an identical efficacy of nitrated fatty acids can be assumed for different settings.

Cytotoxic effects of nitro-fatty acids haven't been described yet.

### Implants

Soft tissue implants are used in a variety of cosmetic, plastic, and reconstructive surgical procedures and may be delivered to many different parts of the body, including, without limitation, the face, nose, jaw, breast, chin, buttocks, chest, lip, and cheek. Soft tissue implants are used for the reconstruction of surgically or traumatically created tissue voids, augmentation of tissues or organs, contouring of tissues, the restoration of bulk to aging tissues, and to correct soft tissue folds or wrinkles (rhytides). Soft tissue implants may be used for the augmentation of tissue for cosmetic (aesthetic) enhancement or in association with reconstructive surgery following disease or surgical resection. Representative examples of soft tissue implants that can be coated with, or otherwise constructed to contain and/or release fibrosis-inhibiting agents provided herein, include, e.g., saline breast implants, silicone breast implants, triglyceride-filled breast implants, chin and mandibular implants, nasal implants, cheek implants, lip implants, and other facial implants, pectoral and chest implants, malar and submalar implants, and buttocks implants. Soft tissue implants have numerous constructions and may be formed of a variety of materials, such as to conform to the surrounding anatomical structures and characteristics. In one aspect, soft tissue implants suitable for combining with a fibrosis-inhibitor are formed from a polymer such as silicone, poly(tetrafluoroethylene), polyethylene, polyurethane, polymethylmethacrylate, polyester, polyamide and polypropylene. Soft tissue implants may be in the form shell (or envelope) that is filled with a fluid material such as saline. In one aspect, soft tissue implants include or are formed from silicone or dimethylsiloxane. Silicone implants can be solid, yet flexible and very durable and stable. They are manufactured in different durometers (degrees of hardness) to be soft or quite hard, which is determined by the extent of polymerization. Short polymer chains result in liquid silicone with less viscosity, while lengthening the chains produces gel-type substances, and cross-linking of the polymer chains results in high-viscosity silicone rubber. Silicone may also be mixed as a particulate with water and a hydrogel carrier to allow for fibrous tissue ingrowth. These implants are designed to enhance soft tissue areas rather than the underlying bone structure. In certain aspects, silicone-based implants (e.g., chin implants) may be affixed to the underlying bone by way of one or several titanium screws. Silicone implants can be used to augment tissue in a variety of locations in the body, including, for example, breast, nasal, chin, malar (e.g., cheek), and chest/pectoral area. Silicone gel with low viscosity has been primarily used for filling breast implants, while high viscosity silicone is used for tissue expanders and outer shells of both saline-filled and silicone-filled breast implants.

In another aspect, soft tissue implants include or are formed from poly(tetrafluoroethylene) (PTFE). In certain aspects, the poly(tetrafluoroethylene) is expanded polytetrafluoroethylene (ePTFE).

In yet another aspect, soft tissue implants include or are formed from polyethylene. Polyethylene implants are frequently used, for example in chin augmentation. Polyethylene implants can be porous, such that they may become integrated into the surrounding tissue. Polyethylene implants may be available with varying biochemical properties, including chemical resistance, tensile strength, and hardness. Polyethylene implants may be used for facial reconstruction, including malar, chin, nasal, and cranial implants.

In yet another aspect, soft tissue implants include or are formed from polypropylene. Polypropylene implants are a loosely woven, high density polymer having similar properties to polyethylene.

In yet another aspect, soft tissue implants include or are formed from polyamide. Polyamide is a nylon compound that is woven into a mesh that may be implanted for use in facial reconstruction and augmentation. These implants are easily shaped and sutured and undergo resorption over time.

In yet another aspect, soft tissue implants include or are formed from polyester. Nonbiodegradable polyesters may be suitable as implants for applications that require both tensile strength and stability, such as chest, chin and nasal augmentation.

In yet another aspect, soft tissue implants include or are formed from polymethylmethacrylate. These implants have a high molecular weight and have compressive strength and rigidity even though they have extensive porosity. Polymethylmethacrylate may be used for chin and malar augmentation as well as craniomaxillofacial reconstruction.

In yet another aspect, soft tissue implants include or are formed from polyurethane. Polyurethane may be used as a foam to cover breast implants. This polymer promotes tissue ingrowth resulting in low capsular contracture rate in breast implants. Commercially available poly(tetrafluoroethylene) soft tissue implants suitable for use in combination with a fibrosis-inhibitor include poly(tetrafluoroethylene) cheek, chin, and nasal implants.

Preferred materials for implants are non-bioabsorbable polymers of natural or synthetic origin. Examples of suitable non-bioabsorbable polymers include, but are not limited to fluorinated polymers (e.g. fluoroethylenes, propylenes, fluoroPEGs), polyolefins such as polyethylene, polyesters such as poly ethylene terepththalate (PET), polypropylene, cellulose, polytetrafluoroethylene (PTFE), nylons, polyamides, polyurethanes, silicones, ultra high molecular weight polyethylene (UHMWPE), polybutesters, polyaryletherketone, copolymers and combinations thereof, poly(tetrafluorethylene) (ePTFE), polymethylmethacrylate, polyester or a polysaccharide, wherein the polysaccharide is glycosaminoglycan.

Other preferred materials are organosilane or organosilicate, carbon-composite, titanium, tantalum, carbon, calcium phosphate, zirconium, niobium, hafnium, hydroxyapatite.

Amphiphilic compound may be linear, branched, block or graft copolymers. The hydrophilic portions are derived from hydrophilic polymers or compounds selected from the member consisting of polyamides, polyethylene oxide, hydrophilic polyurethanes, polylactones, polyimides, polylactams, poly-vinyl-pyrrolidone, polyvinyl alcohols, polyacrylic acid, polymethacrylic acid, poly(hydroxyethyl methacrylate), gelatin, dextran, oligosaccharides, such as chitosan, hyaluronic acid, alginate, chondroitin sulfate, mixtures and combinations thereof. The hydrophobic portions are derived from hydrophobic polymers or compounds selected from the member consisting of polyethylene, polypropylene, hydrophobic polyurethanes, polyacrylates, polymethacrylates, fluoropolymers, polycaprolactone, polylactide, polyglycolide, phospholipids, and polyureas, polyethylene/-vinyl acetate), polyvinylchloride, polyesters, polyamides, polycarbonate, polystyrenes, polytetrafluoroethylene, silicones, siloxanes, fatty acids, and chitosan having high degrees of acetylation and mixtures and combinations thereof. The amphiphilic compound may include any biocompatible combination of hydrophilic and hydrophobic portions.

### Autogenous Tissue Implants

Autogenous tissue implants includes, without limitation, adipose tissue, autogenous fat implants, dermal implants, dermal or tissue plugs, muscular tissue flaps and cell extraction implants. Adipose tissue implants may also be known as autogenous fat implants, fat grafting, free fat transfer, autologous fat transfer/transplantation, dermal fat implants, liposculpture, lipostructure, volume restoration, micro-lipoinjection and fat injections.

Autogenous tissue implants may be also composed of pedicle flaps that typically originate from the back (e.g., latissimus dorsi myocutaneous flap) or the abdomen (e.g., transverse rectus abdominus myocutaneous or TRAM flap). Pedicle flaps may also come from the buttocks, thigh or groin.

The autogenous tissue implant may be also a suspension of autologous dermal fibroblasts that may be used to provide cosmetic augmentation. This method is used for correcting cosmetic and aesthetic defects in the skin by the injection of a suspension of autologous dermal fibroblasts into the dermis and subcutaneous tissue subadjacent to the defect. Typical defects that can be corrected by this method include rhytids, stretch marks, depressed scars, cutaneous depressions of non-traumatic origin, scaring from acne vulgaris, and hypoplasia of the lip. The fibroblasts that are injected are histocompatible with the subject and have been expanded by passage in a cell culture system for a period of time in protein free medium.

The autogenous tissue implant may be also a dermis plug harvested from the skin of the donor after applying a laser beam for ablating the epidermal layer of the skin, thereby exposing the dermis and then inserting this dermis plug at a site of facial skin depressions. This autogenous tissue implant may be used to treat facial skin depressions, such as acne scar depression and rhytides. Dermal grafts have also been used for correction of cutaneous depressions where the epidermis is removed by dermabrasion.

### Surgical Meshes

Surgical meshes can be manufactured for example as hernia mesh, stress urinary incontinence slings, vaginal prolapse suspenders, wound dressing, molded silicone reinforcement, catheter anchoring, pacemaker lead fixation, suture pledgets, suture line buttresses, septal defect plugs, catheter cuffs.

Usual polymers for surgical meshes are polypropylene (filament diameters range from 0.08 mm to 0.20 mm, pore sizes from about 0.8 mm to 3.0 mm, and weights from 25 to 100 gsm), polyester (pore sizes from about 0.5 to 2.0 mm and weights from about 14 to 163 gsm), polytetrafluoroethylene (pore sizes from about 0.8 to 3.5 mm and weights from about 44 to 98 gsm), Polyester Needle Felt (PETNF) (range from 203 to 322 gsm), Polytetrafluoroethylene Needle Felt (PTFENF) (weights of 900 and 1800 gsm) and Dacron (polyethylene terephthalate).

Polypropylene and polytetrafluoroethylene meshes are used for hernia meshes, stress urinary incontinence slings and vaginal prolapse suspenders. Polyester meshes are used for as hernia meshes, wound dressing, molded silicone reinforcement, catheter anchoring and pacemaker lead fixation. PETNF and PTEFENF meshes are used for suture pledgets, suture line buttresses, septal defect plugs and catheter cuffs.

Thus the invention relates to medical devices or implants coated with at least one nitrocarboxylic acid of the general formula (X)

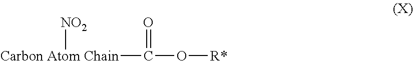

wherein
O—R* represents —OH, polyethylene glycolyl, polypropylene glycolyl, cholesteroyl, phytosteroyl, ergosteroyl, coenzyme A or an alkoxy group consisting of 1 to 10 carbon atoms, wherein this alkoxy group may contain one or more double and/or one or more triple bonds and/or may be substituted by one or more nitro groups and/or one or more substituents S1-S20,
carbon atom chain refers to an alkyl chain to which at least one nitro group is attached consisting of 1 to 40 carbon atoms, wherein this alkyl chain may contain one or more double and/or one or more triple bonds and may be cyclic and/or may be substituted by one or more nitro groups and/or one or more substituents S1-S20, S1-S20 represent independently of each other —OH, —OP(O)(OH)2, —P(O)(OH)2, —P(O)(OCH3)2, —OCH3, —OC2H5, —OC3H7, —O-cyclo-C3H5, —OCH(CH3)2, —OC(CH3)3, —OC4H9, —OPh, —OCH2-Ph, —OCPh3, —SH, —SCH3, —SC2H5, —F, —Cl, —Br, —I, —CN, —OCN, —NCO, —SCN, —NCS, —CHO, —COCH3, —COC2H5, —COC3H7, —CO-cyclo-C3H5, —COCH(CH3)2, —COC(CH3)3, —COOH, —COOCH3, —COOC2H5, —COOC3H7, —COO-cyclo-C3H5, —COOCH(CH3)2, —COOC(CH3)3, —OOC—CH3, —OOC—C2H5, —OOC—C3H7, —OOC-cyclo-C3H5, —OOC—CH(CH3)2, —OOC—C(CH3)3, —CONH2, —CONHCH3, —CONHC2H5, —CONHC3H7, —CON(CH3)2, —CON(C2H5)2, —CON(C3H7)2, —NH2, —NHCH3, —NHC2H5, —NHC3H7, —NH-cyclo-C3H5, —NHCH(CH3)2, —NHC(CH3)3, —N(CH3)2, —N(C2H5)2, —N(C3H7)2, —N(cyclo-C3H5)2, —N[CH(CH3)2]2, —N[C(CH3)3]2, —SOCH3, —SOC2H5, —SOC3H7, —SO2CH3, —SO2C2H5, —SO2C3H7, —SO3H, —SO3CH3, —SO3C2H5, —SO3C3H7, —OCF3, —OC2F5, —O—COOCH3, —O—COOC2H5, —O—COOC3H7, —O—COO-cyclo-C3H5, —O—COOCH(CH3)2, —O—COOC(CH3)3, —NH—CO—NH2, —NH—CO—NHCH3, —NH—CO—NHC2H5, —NH—CO—N(CH3)2, —NH—CO—N(C2H5)2, —O—CO—NH2, —O—CO—NHCH3, —O—CO—NHC2H5, —O—CO—NHC3H7, —O—CO—N(CH3)2, —O—CO—N(C2H5)2, —O—CO—OCH3, —O—CO—OC2H5, —O—CO—OC3H7, —O—CO—O-cyclo-C3H5, —O—CO—OCH(CH3)2, —O—CO—OC(CH3)3, —CH2F, —CHF2, —CF3, —CH2Cl, —CH2Br, —CH2I, —CH2-CH2F, —CH2-CHF2, —CH2-CF3, —CH2-CH2Cl, —CH2-CH2Br, —CH2-CH2I, —CH3, —C2H5, —C3H7, -cyclo-C3H5, —CH(CH3)2, —C(CH3)3, —C4H9, —CH2-CH(CH3)2, —CH(CH3)-C2H5, —C5H11, -Ph, —CH2-Ph, —CPh3, —CH═CH2, —CH2-CH═CH2, —C(CH3)═CH2, —CH═CH—CH3, —C2H4-CH═CH2, —CH═C(CH3)2, —C≡CH, —C═C—CH3, —CH2-C≡CH, —P(O)(OC2H5)2, cholesteryl, nucleotides, lipoamines, dihydrolipoamines, lysobiphospatidic acid, anandamide, long chain N-acyl-ethanolamide, sn-1 substituents with glycerol or diglycerol, sn-2 substituents with glycerol or diglycerol, sn-3 substituents, ceramide, sphingosine, ganglioside, galactosylceramide or aminoethylphosphonic acid.

It is particularly preferred if the at least one nitrocarboxylic acid used for coating the medical device is selected from 12-nitro-linoleic acid, 9-nitro cis-oleic acid, 10-nitro-cis-linoleic acid, 10-nitro-cis-oleic acid, 5-nitro-eicosatrienoic acid, 16-nitro-all-cis-4,7,10,13,16-docosapentaenoic acid, 9-nitro-all-cis-9-12,15-octadecatrienoic acid, 14-nitro-all-cis-7,10,13,16,19-docosapentaenoic acid, 15-nitro-cis-15-tetracosenoic acid, 9-nitro-trans-oleic acid, 9,10-nitro-cis-oleic acid, 13-nitro-octadeca-9,11,13-trienoic acid, 10-nitro-trans-oleic acid, 9-nitro-cis-hexadecenoic acid, 11-nitro-5,8,11,14-eicosatrienoic acid, 9,10-nitro-trans-oleic acid, 9-nitro-9-trans-hexadecenoic acid, 13-nitro-cis-13-docosenoic acid, 8,14-nitro-cis-5,8,11,14-eicosatetraenoic acid, 4,16-nitro-docosahexaenoic acid, 9-nitro-cis-6,9,12-octadecatrienoic acid, 6-nitro-cis-6-octadecenoic acid, 11-nitro-cis-5,8,11,14-eicosatetraenoic acid and combinations thereof.

It is also particularly preferred if the nitrocarboxylic acid is derived from hexanoic acid, octanoic acid, decanoic acid, dodecanoic acid, tetradecanoic acid, hexadecanoic acid, heptadecanoic acid, octadecanoic acid, eicosanoic acid, docosanoic acid, tetracosanoic acid, cis-9-tetradecenoic acid, cis-9-hexadecenoic acid, cis-6-octadecenoic acid, cis-9-octadecenoic acid, cis-11-octadecenoic acid, cis-9-eicosenoic acid, cis-11-eicosenoic acid, cis-13-docosenoic acid, cis-15-tetracosenoic acid, t9-octadecenoic acid, t11-octadecenoic acid, t3-hexadecenoic acid, 9,12-octadecadienoic acid, 6,9,12-octadecatrienoic acid, 8,11,14-eicosatrienoic acid, 5,8,11,14-eicosatetraenoic acid, 7,10,13,16-docosatetraenoic acid, 4,7,10,13,16-docosapentaenoic acid, 9,12,15-octadecatrienoic acid, 6,9,12,15-octadecatetraenoic acid, 8,11,14,17-eicosatetraenoic acid, 5,8,11,14,17-eicosapentaenoic acid, 7,10,13,16,19-docosapentaenoic acid, 4,7,10,13,16,19-docosahexaenoic acid, 5,8,11-eicosatrienoic acid, 9c 11t 13t eleostearic acid, 8t 10t 12c calendic acid, 9c 11t 13c catalpic acid, 4, 7, 9, 11, 13, 16, 19 docosaheptadecanoic acid, taxoleic acid, pinolenic acid, sciadonic acid, 6-octadecynoic acid, t11-octadecen-9-ynoic acid, 9-octadecynoic acid, 6-octadecen-9-ynoic acid, t10-heptadecen-8-ynoic acid, 9-octadecen-12-ynoic acid, t7,t11-octadecadiene-9-ynoic acid, t8,t10-octadecadiene-12-ynoic acid, 5,8,11,14-eicosatetraynoic acid, retinoic acid, isopalmitic acid, pristanic acid, phytanic acid, 11,12-methyleneoctadecanoic acid, 9,10-methylenhexadecanoic acid, coronaric acid, (R,S)-lipoic acid, (S)-lipoic acid, (R)-lipoic acid, 6,8-bis(methylsulfanyl)-octanoic acid, 4,6-bis(methylsulfanyl)-hexanoic acid, 2,4-bis(methylsulfanyl)-butanoic acid, 1,2-dithiolane carboxylic acid, (R,S)-6,8-dithiane octanoic acid, (R)-6,8-dithiane octanoic acid, (S)-6,8-dithiane octanoic acid, tariric acid, santalbic acid, stearolic acid, 6,9-octadecenynoic acid, pyrulic acid, crepenynic acid, heisteric acid, t8,t10-octadecadiene-12-inoic acid. ETYA, cerebronic acid, hydroxynervonic acid, ricinoleic acid, lesquerolic acid, brassylic acid and thapsic acid.

### Findings

Examples 2, 3, 7, 9, and 11 show the efficacy of nitrated fatty acids to inhibit the perception of physical stressors as well of the major exogenous mediators that potentiate the stimulatory effects of an irritant and are able to induce the proliferation of fibroblasts as well as the production of extracellular matrix. Both conditions prevail in clinical situations that include medical treatments such as surgical, plastic, or cosmetic procedures, thus causing injuries, wherein said irritation or injury is selected from cut, tear, dissection, resection, suture, wound closure, debridgement, cauterization, suction, drainage, implantation, grafting, or results from an interventional procedure, wherein this interventional procedure is selected from aspiration of the bile and pancreas ducts, esophagus, or intestines.

### Examples 3, 7, 8, and 10

generate compelling evidence that nitrated fatty acids suppress the nociception and stimulus perception of macrophages and fibroblasts from sensing of artificial surfaces, thereby inhibiting the key events that would otherwise lead to foreign body formation. Thereby, additional fibrosis stimuli are also eliminated. In combination with the effects described in examples 1, 9 and 11 where inhibitory effects of nitrated fatty acids on fibroblasts exposed to chemokines have been observed, these results showed effectiveness in the suppression of a nonphysiological or pathological response in a clinical condition where a medical device such as wound coating and bandage material, suture material, surgical instruments, clinical gloves, injection needles, helices, cannulae, tubes, hip implants, materials for osteosynthesis, medical cellulose, bandaging materials, wound inserts, tissue replacement materials, surgical suture materials, compresses, sponges, medical textiles, ointments, gels, film-building sprays, or meshes are brought into temporary or permanent intimate contact with tissues. As a result, it is justified to state that a surface with a nitrated fatty acid coating improves biocompatibility.

### Examples 4, 6, 9, and 10

justify the assumption that in clinical situations in which endogenous or exogenous exposure to toxins, chemokines and/or irritants occur, nonphysiologic or pathologic reactions of mast cells can be inhibited. Since mast cell activation is a further key event in fibrosis induction, mast cell stabilization is capable of avoiding secondarily caused diseases. Therefore, nitrated fatty acids can be used for various clinical conditions and diseases such as osteomyelofibrosis, chronic polyarthritis, atrophia of mucuous tissues or epidermis, dermatitis ulcerosa, connective tissue diseases such dermatomyositis, chronic vasculitis, polyarteritis nodosa, Buerger's disease, non-tropical sprue, induratio fibroplastica penis, prostate hypertrophy; as well as diseases with an inflammatory component such as enteropathies like tropical sprue or coeliac disease, or from bronchiectasis, emphysema, chronic obstructive pulmonary disease (COPD), dermatoses such as atrophic contact dermatosis, or from gouty arthritis, osteoarthrosis, degenerative arthrotic conditions, toxic shock syndrome, amyolidosis, dermatitis ulcerosa and nephrosclerosis, cystic fibrosis, atopic dermatose, atrophy of mucuous tissue or epidermis, connective tissue diseases such as Sharp syndrome and dermatomyositis, aphthous ulcer, Stevens-Johnson syndrome and toxic epidermal necrolysis.

### Example 11

outlines the suppression of a key mediator by nitrated fatty acids that is responsible nonphysiologic and pathologic formation of extracellular matrix proteins which is the main constituent and/or responsible for dys/malfunction and/or symptoms in various clinical conditions and diseases. In such situations, further reduction of adverse effects can be assumed by the inhibitory effects of nitrated fatty acids to irritants on the migration and proliferation of fibroblasts as supported by the results of examples 1, 3, and 8. Such conditions and/or diseases include but are not restricted to exogenous irritation like wounding or trauma, organ infarctions, hypothermia, burn, chemical burn, alkali burn, burning frostbite, cauterization, granuloma, necrosis, ulcer, fracture, foreign body reaction, cut, scratch, laceration, bruise, tear, contusion, fissuring, burst, or acute or chronic physical, chemical or electrical irritation including fascitis, tendonitis, or prostate hypertophy, induratio fibroplastica penis, myocardial hypertrophy.

### Example 5

gives compelling evidence that a key mechanism of action of nitrated fatty acids relies on the reduction or inhibition of membrane protein perception and signal transduction. Since the TRPV-1 receptor is a representative of receptors responsible for nociception, modulation of nonphysiologic or pathologic irritation of nocicepive receptors by nitrated fatty acids is shown. Therefore, nitrated fatty acids can be used in a clinical condition and/or disease in which nociception is caused by endogenous or exogenous irritants. Such conditions are likewise wounding or trauma, organ infarction, poisoning, hypothermia, burn, chemical burn, alkali burn, burning frostbite, cauterization, necrosis, ulcer, fracture, cut, scratch, laceration, bruise, tear, contusion, fissuring, burst, or chronic physical, chemical or electrical irritations like fascitis, tendonitis, neuropathy, acute or chronic pain, hypersensitivity syndrome, neuropathic pain, atopies such as urticaria, allergic rhinitis and hay fever, enteropathies such as tropical sprue or coeliac disease.

### Application Modes and Pharmaceutical Compositions

According to the invention nitrocarboxylic acids shall be used as therapeutic agents for the treatment and prophylaxis of aggressive cell responses, respectively such a healing pattern. In order to apply the inventive agent to the organism of a mammal including humans as a drug a suitable pharmaceutical composition is required.

According to the described effects of nitrocarboxylic acids on cells and organelles and as set forth in the examples there is a variety of clinical settings in which nitrocarboxylic acids reduce aggressive cell responses. According to the invention nitrocarboxylic acids can be used as a passive coating on materials brought in intimate contact with affected tissues. The amount of nitrocarboxylic acids brought onto the surface of foreign materials for biopassivation is too low to show pharmacological effects.

However, the inventive physical und physicochemical interactions between nitrocarboxylic acids at the interface with foreign materials and adhering cells result in an absence of a contact activation of the cell due to such a stimulus. Hence, the major driving force for the development of an aggressive healing pattern is diminished without necessity for nitrocarboxylic acids to partitionate in cell layers distant from the interphase plane. Therefore such an application mode can be used for biopassivation without triggering pharmacological actions. In other clinical settings a locally restricted partition of nitrocarboxylic acids to cover the affected cells is needed. The concentrations required for an effective reduction of an aggressive healing pattern are also below the threshold for pharmaceutical actions. Additionally, nitrocarboxylic acids may be used as therapeutic agents for the treatment and prophylaxis of such a healing pattern. In order to apply the inventive agent to the organism of a mammal including humans a suitable pharmaceutical composition is required.

Such compositions comprise the nitrocarboxylic acid as an active or passive ingredient or a combination of at least one nitrocarboxylic acid together with at least one further active agent, together with at least one pharmaceutically acceptable carrier, excipient, binders, disintegrates, glidents, diluents, lubricants, coloring agents, sweetening agents, flavoring agents, preservatives or the like. The pharmaceutical compositions of the present invention can be prepared in a conventional solid or liquid carrier or diluents and a conventional pharmaceutically-made adjuvant at suitable dosage level in a known way. If the pharmaceutical composition comprises two nitrocarboxylic acid compounds they are contained preferably in the combination in an amount from 20% by weight of compound 1 to 80% by weight of compound 2 to 80% by weight of compound 1 to 20% by weight of compound 2. More preferably, the two compounds are contained in the combination in an amount from 30% by weight of compound 1 to 70% by weight of compound 2 to 70% by weight of compound 1 to 30% by weight of compound 2. Still more preferably the two compounds are contained in the combination in an amount from 40% by weight of compound 1 to 60% by weight of compound 2 to 60% by weight of compound 1 to 40% by weight of compound 2.

Preferably the at least one nitrocarboxylic acid is suitable for intravenous, intraarterial, intraperitoneal, interstitial, intrathecal administration, instillation, infiltration, apposition, suitable for ingestion, respectively oral administration or suitable for administration by inhalation.

Administration forms include, for example, pills, tablets, film tablets, coated tablets, capsules, liposomal formulations, micro- and nano-formulations, powders and deposits. Furthermore, the present invention also includes pharmaceutical preparations for parenteral application, including dermal, intradermal, intragastral, intracutan, intravasal, intraarterial, intravenous, intramuscular, intraperitoneal, intranasal, intravaginal, intrabuccal, percutan, rectal, subcutaneous, sublingual, topical, or transdermal application, which preparations in addition to typical vehicles and/or diluents contain the peptide or the peptide combination according to the present invention.

The present invention also includes mammalian milk, artificial mammalian milk as well as mammalian milk substitutes as a formulation for oral administration of the peptide combination to newborns, toddlers, and infants, either as pharmaceutical preparations, and/or as dietary food supplements.

The pharmaceutical compositions according to the present invention will typically be administered together with suitable carrier materials selected with respect to the intended form of administration, i.e. for oral administration in the form of tablets, capsules (either solid filled, semi-solid filled or liquid filled), powders for constitution, aerosol preparations consistent with conventional pharmaceutical practices. Other suitable formulations are gels, elixirs, dispersible granules, syrups, suspensions, creams, lotions, solutions, emulsions, suspensions, dispersions, and the like. Suitable dosage forms for sustained release include tablets having layers of varying disintegration rates or controlled release polymeric matrices impregnated with the active components and shaped in tablet form or capsules containing such impregnated or encapsulated porous polymeric matrices. The pharmaceutical compositions may be comprised of 5 to 95% by weight of the at least one nitrocarboxylic acid, while also up to 100% of the pharmaceutical composition can consist of the at least one nitrocarboxylic acid.

As pharmaceutically acceptable carrier, excipient and/or diluents can be used lactose, starch, sucrose, cellulose, magnesium stearate, dicalcium phosphate, calcium sulfate, talc, mannitol, ethyl alcohol (liquid filled capsules), albumin, PEG. HES, amino acids such as arginine, cholesteryl esther, liquid crystals, zeolites.

Suitable binders include starch, gelatin, natural sugars, cyclodextrins, corn sweeteners, natural and synthetic gums such as acacia, sodium alginate, carboxymethyl-cellulose, polyethylene glycol and waxes. Among the lubricants that may be mentioned for use in these dosage forms, boric acid, sodium benzoate, sodium acetate, sodium chloride, and the like. Disintegrants include starch, methylcellulose, guar gum and the like. Sweetening and flavoring agents and preservatives may also be included where appropriate. Some of the terms noted above, namely disintegrants, diluents, lubricants, binders and the like, are discussed in more detail below.

Additionally, the compositions of the present invention may be formulated in sustained release form to provide the rate controlled release of any one or more of the components or active ingredients to optimize the therapeutic effects. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

Aerosol preparations suitable for inhalation may include solutions and solids in powder form, which may be in combination with a pharmaceutically acceptable carrier such as inert compressed gas, e.g. nitrogen.

For preparing suppositories, a low melting wax such as a mixture of fatty acid glycerides such as cocoa butter is first melted, and the active ingredient is dispersed homogeneously therein by stirring or similar mixing. The molten homogeneous mixture is then poured into convenient sized molds, allowed to cool and thereby solidify.

Also included are solid form preparations which are intended to be converted, shortly before use, to liquid form preparations for either oral or parenteral administration. Such liquid forms include solutions, suspensions and emulsions.

The at least one nitrocarboxylic acid of the present invention may also be deliverable transdermally. The transdermal compositions may take the form of creams, lotions, aerosols and/or emulsions and can be included in a transdermal patch of the matrix or reservoir type as are conventional in the art for this purpose.

The transdermal formulation of the at least one nitrocarboxylic acid of the invention is understood to increase the bioavailability of said nitrocarboxylic acid in the circulating blood or in subcutaneous tissues. One problem in the administration of nitrocarboxylic acid(s) is the loss of bioactivity due to the formation of insolubles in aqueous environments or due to degradation. Therefore stabilization of the nitrocarboxylic acid(s) for maintaining their fluidity and maintaining their biological activity upon administration to the patients in need thereof needs to be achieved. Prior efforts to provide active agents for medication include incorporating the medication in a polymeric matrix whereby the active ingredient is released into the systemic circulation. Known sustained-release delivery means of active agents are disclosed, for example, in U.S. Pat. No. 4,235,988, U.S. Pat. No. 4,188,373, U.S. Pat. No. 4,100,271, U.S. Pat. No. 447,471, U.S. Pat. No. 4,474,752, U.S. Pat. No. 4,474,753, or U.S. Pat. No. 4,478,822 relating to polymeric pharmaceutical vehicles for delivery of pharmaceutically active chemical materials to mucous membranes. The pharmaceutical carriers are aqueous solutions of certain polyoxyethylene-polyoxypropylene condensates. These polymeric pharmaceutical vehicles are described as providing for increased drug absorption by the mucous membrane and prolonged drug action by a factor of two or more. The substituents are block copolymers of polyoxypropylene and polyoxyethylene used for stabilization of drugs.

Aqueous solutions of polyoxyethylene-polyoxypropylene block copolymers (poloxamers) with or without a plasticizer are useful as stabilizers for peptide(s). Poloxamers, also known by the trade name Pluronics (e.g. Pluronic F127, Pluronic P85, Pluronic F68) have surfactant properties that make them useful in industrial applications. In a preferred embodiment the preparation is provided in form of a nanogel.

The term capsule refers to a special container or enclosure made of methyl cellulose, polyvinyl alcohols, or denatured gelatines or starch for holding or containing compositions comprising the active ingredients. Hard shell capsules are typically made of blends of relatively high gel strength bone and pork skin gelatins. The capsule itself may contain small amounts of dyes, opaquing agents, plasticizers and preservatives.

Tablet means compressed or molded solid dosage form containing the active ingredients with suitable diluents. The tablet can be prepared by compression of mixtures or granulations obtained by wet granulation, dry granulation or by compaction well known to a person skilled in the art.

Oral gels refer to the active ingredients dispersed or solubilized in a hydrophilic or hydrophobic semi-solid matrix.

Powders for constitution refer to powder blends containing the active ingredients and suitable diluents which can be suspended in water or juices. One example for such an oral administration form for newborns, toddlers and/or infants is a human breast milk substitute which is produced from milk powder and milk whey powder, optionally and partially substituted with lactose.

Suitable diluents are substances that usually make up the major portion of the composition or dosage form. Suitable diluents include sugars such as lactose, sucrose, mannitol and sorbitol, starches derived from wheat, corn rice and potato, and celluloses such as microcrystalline cellulose, lipids, triglycerides, oils, hydrogels like gelatine, organogels. The amount of diluents in the composition can range from about 5 to about 95% by weight of the total composition, preferably from about 25 to about 75%, more preferably from about 30 to about 60% by weight, and most preferably from about 40 to 50% by weight.

The nitrocarboxylic acid(s) of the invention can be used to form multiparticulates, discrete particles, well known dosage forms, whose totality represents the intended therapeutically useful dose of a drug. When taken orally, multiparticulates generally disperse freely in the gastrointestinal tract, and maximize absorption. A specific example is described in U.S. Pat. No. 6,068,859, disclosing multiparticulates that provide controlled release of azithromycin. Another advantage of the multiparticulates is the improved stability of the drug. The poloxamer component of the multiparticulate is very inert, thus minimizing degradation of the drug.

Preferably, the at least one nitrocarboxylic acid can be formulated with a poloxamer and a resin to form micelles suitable for oral administration to patients in need of the drug.

Liquid form preparations include solutions, suspensions, emulsions and liquid crystals. As an example may be mentioned water or water-propylene glycol solutions for parenteral injections or addition of sweeteners and opacifiers for oral solutions, suspensions and emulsions. Liquid form preparations may also include solutions for intranasal administration.

For administration by inhalation the particle diameter of the lyophilised preparation is preferably between 2 to 5 μm, more preferably between 3 to 4 μm. The lyophilised preparation is particularly suitable for administration using an inhalator, for example the OPTINEB® or VENTA-NEB® inhalator (NEBU-TEC, Elsenfeld, Germany). The lyophilised product can be rehydrated in sterile distilled water or any other suitable liquid for inhalation administration.

Alternatively for intravenous administration the lyophilised product can be rehydrated in sterile distilled water or any other suitable liquid for intravenous administration.

The preferred dosage concentration for either intravenous, oral, or inhalation administration is between 100 and 2000 μmol/ml, and more preferably is between 200 and 800 μmol/ml.

### Method of Treatment

The present invention relates to a method of prophylaxis and/or treatment of an aggressive healing pattern or to the attenuation of the response to an irritating stimulus by administering to a patient in need thereof a pharmaceutical composition or a passivating coating of a medical device or implant comprising at least one nitrocarboxylic acid according to the present invention in a therapeutically effective amount to be effective in at least one of the aforementioned clinical conditions or diseases.

The nitrocarboxylic acids of the present invention can be used for the prophylaxis and/or treatment progression due to an irritation/injury/medical manipulation, arising from an aggressive healing pattern or any other disease or state mentioned above in combination administration with another therapeutic compound. As used herein the term “combination administration” of a compound, therapeutic agent or known drug with the nitrocarboxylic acid(s) of the present invention means administration of the drug and the nitrocarboxylic acid(s) at such time that both the known drug and the nitrocarboxylic acid(s) will have a therapeutic effect. In some cases this therapeutic effect will be synergistic. Such concomitant administration can involve concurrent (i.e. at the same time), prior, or subsequent administration of the drug with respect to the administration of the nitrocarboxylic acid(s) of the present invention. A person of ordinary skill in the art would have no difficulty determining the appropriate timing, sequence and dosages of administration for particular drugs of the present invention.

Moreover the present invention relates to a method for modulating a disease displaying an aggressive healing response of tissues, cells or organelles which is not due to a genuine inflammation in a mammal including humans, which comprises administering to the mammal a pharmaceutically effective amount of a nitrocarboxylic acid or salts or hydrates thereof effective to prevent or treat said aggressive healing response.

### DEFINITIONS

The term “aggressive healing process” is defined as a reaction of an organism to physical, electrical, thermal, chemical alteration or trauma of cells or tissues that cause a response of the affected or neighboring cells that initiates migration, differentiation, proliferation or apoptosis of the affected or neighboring cells leading to (1) the formation of extracellular matrix, and/or (2) the accumulation of cells, that (3) each or both goes beyond the amount of material needed to fill the defect, and/or (4) the formation or invasion of cells that impair/disturb/destroy. tissue/organ functionality, and/or (5) cells and/or extracellular matrix structures interconnect/adhere/agglutinate/bake together tissues in an unphysiological pattern, leading to (6) symptoms/impaired tissue or organ functionality, and/or (7) cosmetic or esthetic impairments. The clinical and histological uniform appearance of an aggressive healing process that can be estimated by a person skilled in the art is the presence of either extracellular matrix and/or of proliferated cells which have developed during the healing process and result in an amount of solid material which goes beyond that needed to fill the defect or impair the affected tissues thereby reducing their functionality and/or causing cosmetic/esthetic impairments. Pathophysiological or pathological as used herein refers to all healing patterns which don't take a physiological course and develop at the same time pathologic symptoms that need to be attended medically. In other words, these terms refer to any biochemical, functional or structural reaction in/of a cell, organelle or tissue that is typical for a defined pathology of the given cells or tissues.

Non-physiological as used herein refers in general to all healing patterns which don't take a physiological course but not necessarily have to develop pathologic or other symptoms and therefore only casually need medical attention. In other words, this term refers to any biochemical, functional or structural reaction in/of a cell, organelle or tissue that is not characteristic for the given type of cells or tissues during normal development or function.

The term “irritating stimulus” refers to any exogenous or endogenous stimulus able to provoke a biochemical, functional or structural change in a cell, organelle or tissue that can be characterized as pathophysiological or non-physiological.

The term “response” as used in this context refers to any a biochemical, functional or structural reaction in a cell, organelle or tissue that can be characterized as pathophysiological or non-physiological.

The term “genuine” defines the ethiological affinity to physiological or pathophysiological causes of a clinical condition or disease.

Genuine inflammation or a primary inflammatory disease are defined as clinical conditions where several pathways of the immune system are activated at the same time caused by a bacterial, viral or microbial agents, and in which at least three of the following immunological conditions/reactions are involved

1. infiltration of neutrophiles and lymphocytes (TH-2-like cells)
2. induction of iNOS(NOS-2)
3. production of TNF alpha
4. induction of COX-2
5. induction of 5-lipooxigenase.

The terms “prophylaxis” or “treatment” includes the administration of the nitrocarboxylic acid(s) of the present invention to prevent, inhibit, or arrest symptoms and/or dys-/malfunction and/or esthetic/cosmetic impairment due to an cell/tissue/organ reaction to an irritant, arising from an aggressive healing pattern, pathological or non-physiological reaction. In some instances, treatment with the nitrocarboxylic acid(s) of the present invention will be done in combination with other protective compounds to prevent, inhibit, or arrest the symptoms thereof.

The term “active agent” or “therapeutic agent” as used herein refers to an agent that can prevent, inhibit, or arrest the symptoms and/or progression due to an irritation/injury/medical manipulation, arising from an aggressive healing pattern or any other disease or state mentioned above. Such an agent requires a pharmaceutical preparation or formulation that effects a desired pharmacodynamic distribution within tissues, organs or the whole organism. However, according to the intervention active does not necessarily mean that the agent has to have a specific action on/to one or more specific receptors or other anchoring sites of a cell, neither have to have a direct blocking or activating action to specific intracellular signalling cascades. Moreover, the principal effect is based on a change of the physical or physico-chemical properties of the cell/organelle membrane.

The term “passive agent” as used herein refers to an agent that can prevent, inhibit, or arrest the symptoms and/or progression of an irritation, injury and/or medical manipulation showing an aggressive healing pattern, or any other disease or state mentioned above by reducing nociception, perception of contact activators or passivators like artificial surfaces or toxins, without having a specific affinity to one or more of these cell or tissue sites. The passive agent comes in intimate contact at an interphase with these sites, thereby preventing the pathophysiologic or non-physiologic response of a cell or tissue to an irritating stimulus by interfering with the physical or physicochemical properties of the cell membrane without showing a specific pharmacological action like a receptor activation, and without being present in cell or tissue layers distant from the interphase plane.

The term “therapeutic effect” as used herein, refers to the effective provision of protection effects to prevent, inhibit, or arrest the symptoms and/or progression due to an irritation, injury or medical manipulation, arising from an aggressive healing pattern or any other disease or state mentioned above.

The term “a therapeutically effective amount” as used herein means a sufficient amount of the nitrocarboxylic acid(s) of the invention to produce a therapeutic effect, as defined above, in a subject or patient in need of treatment.

The terms “subject” or “patient” are used herein mean any mammal, including but not limited to human beings, including a human patient or subject to which the compositions of the invention can be administered. The term mammals include human patients and non-human primates, as well as experimental animals such as rabbits, rats, and mice, and other animals.

The nitrocarboxylic acid(s) of the present invention can be used for the prophylaxis and/or treatment progression due to an irritation, injury or medical manipulation, arising from an aggressive healing pattern or any other disease or state mentioned above in combination administration with another therapeutic compound. As used herein the term “combination administration” of a compound, therapeutic agent or known drug with the nitrocarboxylic acid(s) of the present invention means administration of the drug and the nitrocarboxylic acid(s) at such time that both the known drug and the nitrocarboxylic acid(s) will have a therapeutic effect. In some cases this therapeutic effect will be synergistic. Such concomitant administration can involve concurrent (i.e. at the same time), prior, or subsequent administration of the drug with respect to the administration of the nitrocarboxylic acid(s) of the present invention. A person of ordinary skill in the art would have no difficulty determining the appropriate timing, sequence and dosages of administration for particular drugs of the present invention.

### Definition of Nitrocarboxylic Acid Activity

A nitrocarboxylic acid is deemed to have therapeutic activity if it demonstrated any one of the following activities listed in a) to g).

a) The nitrocarboxylic acid could inhibit the activity of an over active biological pathway.
b) The nitrocarboxylic acid(s) could inhibit the production of an over produced biological molecule.
c) The nitrocarboxylic acid could inhibit the activity of an over produced biological molecule.
d) The nitrocarboxylic acid could increase the activity of an under active biological pathway.
e) The nitrocarboxylic acid could increase the production of an under produced biological molecule.
f) The nitrocarboxylic acid could mimic the activity of an under produced biological molecule.
g) The nitrocarboxylic acid could modulate pathophysiologic or non-physiologic cell responses to physiologic, pathologic and non-physiologic stimuli.
h) The nitrocarboxylic acid could stabilize cell/plasma membranes thereby modulating physical and/or biological properties.
i) The nitrocarboxylic acid could prevent, inhibit, or arrest the symptoms and/or progression of an irritation, injury or medical manipulation arising from an aggressive healing pattern.

As used herein “inhibition” is defined as a reduction of the activity or production of a biological pathway or molecule activity of between 10 to 100%. More preferably the reduction of the activity or production of a biological pathway or molecule activity is between 25 to 100%. Even more preferably the reduction of the activity or production of a biological pathway or molecule activity is between 50 to 100%.

As used herein “increase” is defined as an increase of the activity or production of a biological pathway or molecule of between 10 to 100%. More preferably the increase of the activity or production of a biological pathway or molecule activity is between 25 to 100%. Even more preferably the increase of the activity or production of a biological pathway or molecule activity is between 50 to 100%.

As used herein “mimic” is defined as an increase in the activity of a biological pathway dependent on the under produced biological molecule of between 10 to 100%. More preferably the increase of the activity of the biological pathway is between 25 to 100%. Even more preferably the increase of the activity of the biological pathway is between 50 to 100%.

### Coating of Medical Devices and Contact Application of a Nitrocarboxylic Acid

Instant contact application is the preferred method for their preventive and therapeutic use. A preferred embodiment is the coating of a medical device or onto implant surfaces or interphases with at least one nitrocarboxylic acid.

Further substances to be mentioned for application onto a medical device or onto implant surfaces or interphases together with the inventive nitrocarboxylic acids are 2-pyrrolidon, tributylcitrate, triethylcitrate and their acetylated derivatives, bibutylphthalate, benzoic acid benzylester, diethanolamine, diethylphthalate, isopropylmyristate and palmitate, triacetin, DMSO, iodine-containing contrast agents, PETN, isopropylmyristate, isopropylpalmitate and benzoic acid benzylester.

Depending of the target site of the medical device or implant a polymer matrix might be necessary. Therewith, the premature blistering of a pure active agent layer consisting of at least one nitrocarboxylic acid is prevented. Biostable and biodegradable polymers can be used as matrices which are listed below. Especially preferred are polysulfones, polyurethanes, polylactides, parylenes and glycolides and their copolymers.

Moreover the nitrocarboxylic acid can be administered or can be placed on the surface of the medical device or implant together with one or more further active ingredients such as anti-proliferative agents, anti-inflammatory agents, antibiotics, anti-metabolic agents, anti-angiogenic agents, anti-viral agents and/or analgetics.

Another method of nitrocarboxylic acid delivery is a lipid double layer coating of a device. The technique is based on a covalent binding of fatty acids or analogs such as sphingosines on a surface. A preferred group of fatty acids are tetraether lipids. In a second step the nitrocarboxylic acids are spread on the surface by using the so-called Langmuir technique.

Such medical devices which can be used according to the invention can be coated, on the one hand, by applying a coating on the solid material.

The concentration of the at least one nitrocarboxylic acid and of other active agent if present is preferably in the range of 0.001-500 mg per cm² of the completely coated surface of the endoprosthesis, i.e. the surface is calculated taking into consideration the total surface.

The methods according to the invention are adapted for coating for example endoprostheses and in particular non-vascular stents like tracheal stents, bronchial stents, urethral stents, esophageal stents, biliary stents, stents for use in the small intestine, stents for use in the large intestine and other metallic implants.

The invention also refers to polymeric, respectively non-metallic implants, such as polymeric protheses like surgical meshes, pace-makers for the heart or brain, tissue grafts, breast implants, and any other implant for cosmetic or reconstitutionary purposes, particularly silicone-based implants.

Furthermore, this invention refers also to catheters and wirings in general and in particular drainage catheters and electrodes.

This invention also refers to grafts such as allografts, xenografts and homografts.

Moreover, helices, canulas, tubes as well as generally implants, materials for osteosynthesis, medical cellulose, bandaging materials, wound inserts, surgical suture materials, compresses, sponges, medical textiles, ointments, gels or film-building sprays, meshes, fibers or tissues or parts of the above mentioned medical devices can be coated according to the invention.

Materials for such medical products can be selected from the group comprising or consisting of: parylenes (such as parylene C, parylene D, parylene N, parylene F), polyacrylic acid, polyacrylates, polymethylmethacrylate, polybutylmethacrylate, polyisobutylmethacrylate, polyacrylamide, polyacrylnitrile, polyamide, polyetheramide, polyethylenamine, polyimide, polypropylene, polycarbonate, polycarbourethane, polyvinylketone, polyvinyl halogenide, polyvinylidene halogenide, polyvinyl ether, polyvinyl aromates, polyvinyl esters, polyvinyl pyrollidone, polyoxymethylene, polyethylene, polypropylene, polytetrafluorethylene, polyurethane, polyolefin elastomer, polyisobutylene, EPDM gums, fluorosilicon, carboxymethylchitosane, polyethylene terephtalate, polyvalerate, carboxymethyl cellulose, cellulose, rayon, rayon triacetate, cellulose nitrate, cellulose acetate, hydroxyethyl cellulose, cellulose butyrate, poly-4-hydroxy butyrate, cellulose acetate-butyrate, ethylvinyl acetate-copolymer, polysulfone, polyethersulfone, epoxy resin, ABS resins, EPDM gums, silicon prepolymer, silicon, polysiloxane, polyvinyl halogene, cellulose ether, cellulose triacetate, chitosane, chitosane derivatives, polymerizable oils, polyvalerolactones, poly-ε-decalactone, polylactide, polyglycolide, copolymers of polylactides and polyglycolides, poly-ε-caprolactone, polyhydroxy butyric acid, polyhydroxy butyrate, polyhydroxy valerate, polyhydroxy butyrate-co-valerate, poly(1,4-dioxane-2,3-dione), poly(1,3-dioxane-2-one), poly-para-dioxanone, polyanhydride, polymaleic acid anhydride, polyhydroxy methacrylate, polycyanoacrylate, polycaprolactone dimethylacrylate, poly-β-maleic acid, polycaprolactone butyl acrylate, multiblock polymers of oligocaprolactonediol and oligodioxanoendiol, polyetherester-multiblock polymers of PEG and poly(butylene terephtalate), polypivotolactone, polyglycolic acid trimethyl carbonate, polycaprolactone glycolide, poly(γ-ethyl glutamate), poly(DTH-iminocarbonate), Poly(DTE-co-DT-carbonate), Poly(bisphenol A iminocarbonate), polyorthoesters, polyglycolic acid trimethyl carbonate, polytrimethyl carbonate, polyiminocarbonate, polyvinyl alcohols, polyester amides, glycolizated polyesters, polyphosphoesters, polyphosphazenes, poly[-carboxy phenoxy) propane], polyhydroxypentanoic acid, polyethylenoxid propylenoxide, soft polyurethanes, polyurethanes with amino acid residues in the backbone, polyetheresters, polyethylene oxid, polyalkenoxalates, polyorthoesters, carrageenanes, starch, collagen, protein-based polymers, polyamino acids, synthetic polyamino acids, zein, modified zein, polyhydroxy alkanoates, pectinic acid, actinic acid, fibrin, modified fibrin, casein, modified casein, carboxymethyl sulfate, albumine, hyaluronic acid, heparan sulfate, heparin, chondroitine sulfate, dextran, cyclodextrine, copolymers of PEG and polypropylene glycol, gummi arabicum, guar or other gum resins, gelatine, collagen, collagen-N-hydroxy succinimide, lipids, lipoids, polymersizabe oils and their modifications, copolymers and mixtures of the afore-mentioned substances. These materials can also be made of silk, flax or linen. Particularly preferred is the use of parylene (parylene C, parylene D, parylene N, parylene F).

The coated medical devices are preferably used for maintaining the functionality and/or the structure of the treated area or patency of any tubular structure, for example the urinary tract, esophaguses, tracheae, the biliary tract, the renal tract, duodenum, pilorus, the small and the large intestine, but also for maintaining the patency of artificial openings such as used for the colon or the trachea.

Thus, the coated medical devices are useful for preventing, reducing or treating a pathophysiological or non-physiological healing process or an inappropriate or undesirable tissue formation or fusion. This relates to the interventional treatment of tubular structures like the bile duct, oesophagus, or intestines, treatment of any trauma, any type of surgery or tissue suturing or adaptation as well as organ preservation and organ protection.

Another possibility consists in the use of this marginal region as a reservoir for active agents or respectively for introducing active agents especially into this marginal region, wherein these active agents can be different from those possibly present in/on the completely coated surface of the hollow body.

### Materials for Implants and Wound Materials

Implants and especially polymeric implants can be comprised of usual materials, especially polymers, as they are described more below and especially of polyamide such as PA 12, polyester, polyurethane, polyacrylates, polyethers, etc.

As mentioned in the beginning, besides the selection of at least one nitrocarboxylic acid further factors are important to achieve a medical device which is optimally passivation of irritants. The physical and chemical properties of the at least one nitrocarboxylic acid and the optionally added further agent as well as their possible interactions, agent concentration, agent release, agent combination, selected polymers and coating methods represent important parameters which have a direct influence on each other and therefore have to be exactly determined for each embodiment. By regulating these parameters the agent or active combination can be absorbed by the adjacent cells of the dilation site.

On the one hand, the layers can be comprised of pure agent layers, wherein at least one of the layers contains the at least one nitrocarboxylic acid, and on the other hand, of agent-free or active agent-containing polymer layers or combinations thereof.

As methods for manufacturing such a medical device the pipetting method (capillary method), spray method (fold spray method), dipping method, electro-spinning and/or laser technique can be utilized. Depending on the selected embodiment the best-suitable method is selected for the manufacture of the medical device, wherein also the combination of two or more methods can be used.

### General Description of the Coating Methods

### Pipettinq Method—Capillary Method

This method comprises the following steps:
a) providing an implant,
b) providing a coating device with an aperture capable for pointwise release of the coating solution,
c) setting the aperture capable for pointwise release of the coating solution to the proximal or distal end of a fold of the implant, and
d) releasing a defined amount of the coating solution through the outlet at the proximal or distal end of the implant.

Optionally, there can be still step e) for drying:
e) drying of the coating solution wherein the implant is rotated during drying about its longitudinal axis in direction of the aperture of the folds.

This method can be performed with any coating solution which is still so viscous that it is drawn because of capillary forces or by additionally using gravitation into the fold during 5 minutes, preferably 2 minutes, and thus mostly completely fills the fold.

### Spray Method:

This method comprises the following steps:
a) providing an implant,
b) providing a coating device with at least one releasing aperture,
c) positioning the at least one releasing aperture towards the implant surface,
d) release of a defined amount of the coating solution from the at least one releasing aperture onto the implant; and
e) drying of the coating solution on the implant.

Optionally, there can be still step f) for drying:
f) drying of the coating solution or evenly distributing the coating on the implant surface wherein the implant is rotated about its longitudinal axis.

This method can be performed with any coating solution which is still so viscous that it can be sprayed by means of small nozzles or small outlets.

### Dipping Method:

In this method the implant is dipped into a tank or container containing the coating solution. This procedure is repeated until a complete and evenly distributed coating on the implant surface is reached. For a better spread of the coating the implant can optionally be dipped into the tank by continuous variation of its position, for example by a continuous or angle-wise rotation. The dipping method can be combined with a rotation drying described further below.

### Pipetting Method or Capillary Method:

In this method a pipette or a syringe or any other device capable of releasing pointwise the composition containing the active agent is used.

The pipette or syringe or outlet or other device capable for pointwise release of the composition containing the active agent is filled with the composition and its outlet is set preferably to the proximal or distal end of the implant. The escaping composition is drawn from capillary forces along the implant until the opposite end is reached.

### Vapor Deposition Method:

This method includes the following steps:

I) Providing a vacuum chamber,
II) Placing the implant or medical devices in the medical chamber by using holding means,
III) Filling this or these cavities inside the vacuum chamber with the coating solution,
IV) Applying a vacuum to the vacuum chamber,
V) Generating ultrasound in at least one of the cavities containing the coating solution,
VI) Applying the ultrasound-dispersed coating solution on the implant or medical device
VII) Airing the vacuum chamber and removing the implant or medical device.

In this method one or more implants and/or medical devices are placed in a vacuum chamber having at least one cavity containing the coating solution. This at least one cavity is designed in such a way that ultrasound can be generated therein. In this coating method a vacuum of maximally 100 Pa, preferably maximally 10 Pa and particularly preferably maximally 3 Pa is generated. Now ultrasound is generated inside the at least one cavity. The substances contained therein are now dispersed by the ultrasound and are deposited on the objects to be coated. Those parts of the objects that shall not be coated may be covered for protection with an easily removable foil.

It is preferred to lead a slight inert gas flow through the chamber. The gas phase coating can be repeated several times until the desired coating thickness is obtained. This coating method is particularly suitable for implants and medical devices having a porous surface.

### FIGURES

FIG. 1: Nitrocarboxylic acid formation by free radical reactions

FIG. 2: Nitration reactions under high oxygen tensions

FIG. 3: Nitrocarboxylic acid formation by electrophilic substitution

FIG. 4: PhSeBr-catalyzed nitration of alkenes

FIG. 5: Formation of nitrated carboxylic acid esters

FIG. 6: Fibroblasts within an uncoated mesh at day 7,21, and after 8 weeks (a-c), and fibroblasts within a mesh coated with nitro-linoleic acid at day 7, 21, and after 8 weeks (d-f)

FIG. 7: Fibroblasts of cultures with an uncoated (a) and nitrooleic acid coated mesh (b) 21 after 21 days. Fibroblasts within uncoated meshes exhibit a more dentritic shape and more actin-myosin fibers (intra-cellular green filaments) than fibroblasts within the coated meshes. Bar=75 μm.

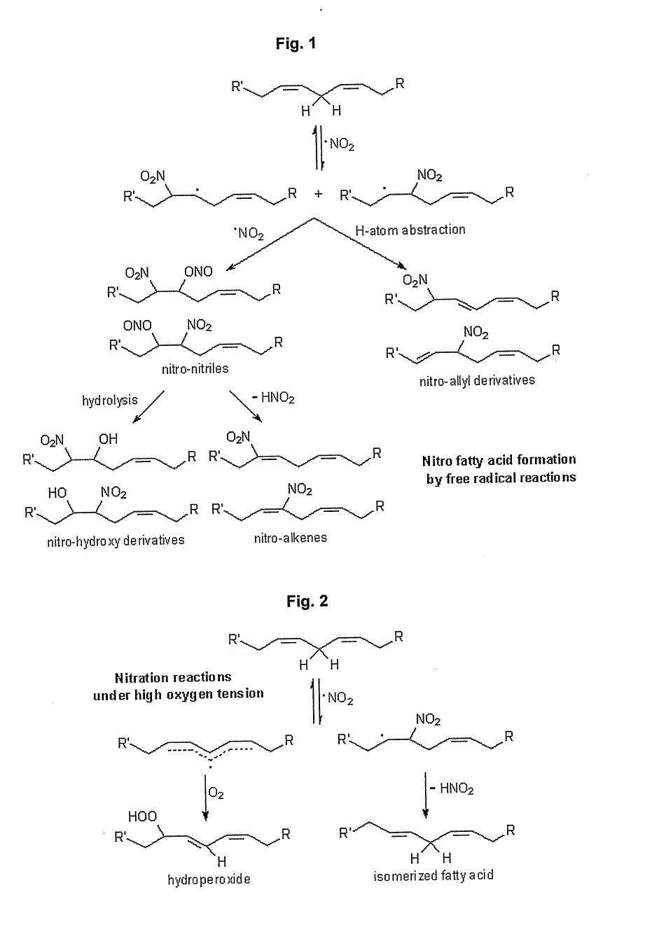

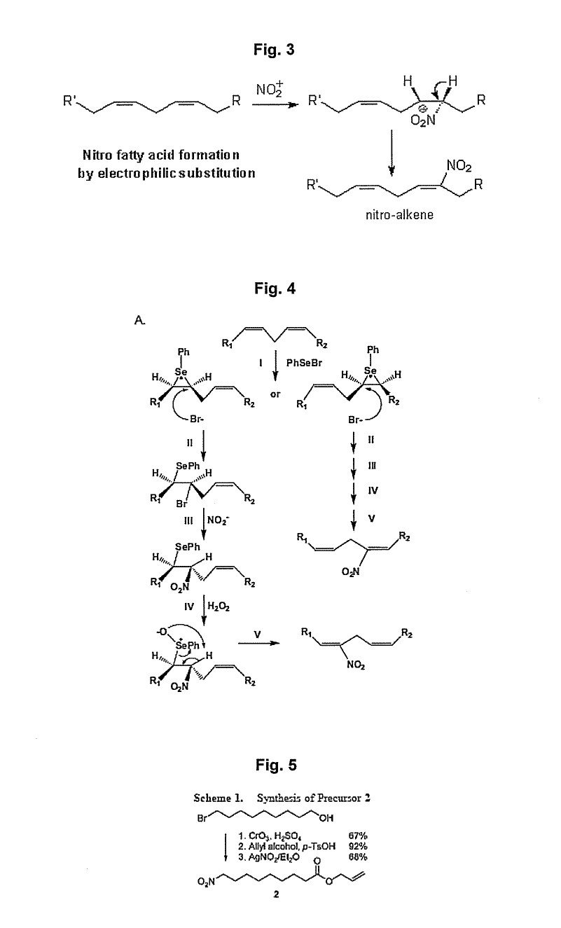

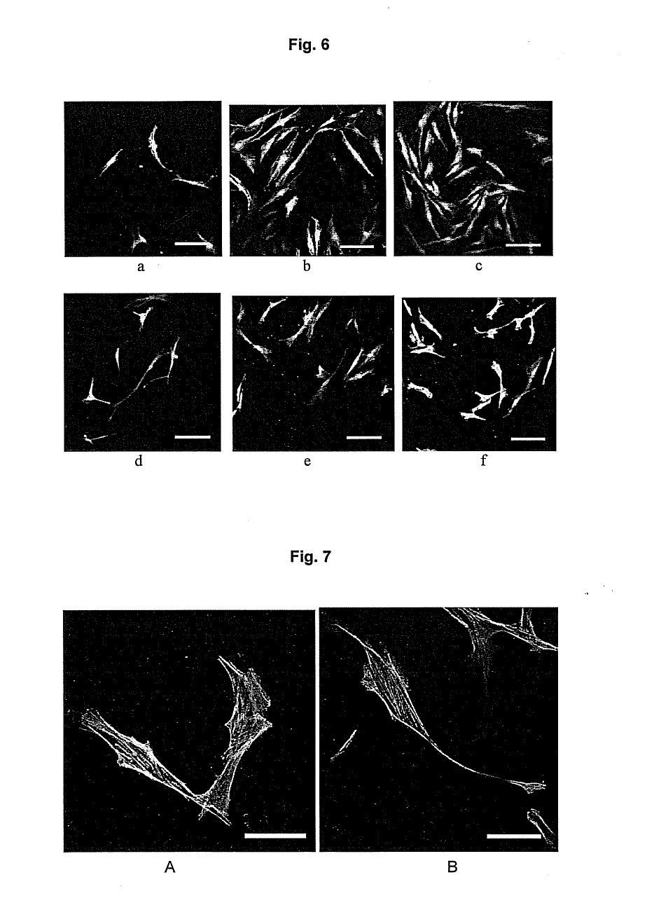

### EXAMPLES

### Table 1 List of all Tested Nitrocarboxylic Acids

1: 9-nitro cis-oleic acid
2: 10-nitro-cis-linoleic acid
3: 10-nitro-cis-oleic acid
4: 5-nitro-eicosatrienoic acid
5: 16-nitro-all-cis-4,7,10,13,16-docosapentaenoic acid (nitro-Osbond acid)
6: 9-nitro-all-cis-9-12,15-octadecatrienoic acid (nitro-linolenic acid)
7: 14-nitro-all-cis-7,10,13,16,19-docosapentaenoic acid (nitro-EPA)
8: 15-nitro-cis-15-tetracosenoic acid (nitro-nervonic acid)
9: 9-nitro-trans-oleic acid
10: 9,10-nitro-cis-oleic acid
11: 13-nitro-octadeca-9,11,13-trienoic acid (nitro-punicic acid)
12: 10-nitro-trans-oleic acid
13: 9-nitro-cis-hexadecenoic acid
14: 11-nitro-5,8,11,14-eicosatrienoic acid
15: 9,10-nitro-trans-oleic acid
16: 9-nitro-9-trans-hexadecenoic acid (nitro-palmitoleic acid)
17: 13-nitro-cis-13-docosenoic acid (nitro-erucic acid)
18: 8,14-nitro-cis-5,8,11,14-eicosatetraenoic acid (dinitro-arachidonic acid)
19: 4,16-nitro-docosahexaenoic acid (nitro-DHA)
20: 9-nitro-cis-6,9,12-octadecatrienoic acid (nitro-GLA)
21: 6-nitro-cis-6-octadecenoic acid (nitro-petroselinic acid)
22: 11-nitro-cis-5,8,11,14-eicosatetraenoic acid (nitro-arachidonic acid)

This set of nitrocarboxylic acids (nitro fatty acids) was tested in all experiments, unless otherwise stated. As controls (native FA; FA refers to fatty acid) the respective non-nitrated nitrocarboxylic acids were used. These compounds are also designed as native fatty acids.

### Example 1

### Investigation to Determine the Effect of Nitrocarboxylic Acids on Biofouling and Cell Adherence at Prosthetic Materials

Polymer scaffolds (polyurethane, polyvinylchloride, polylactate) which are used as implant materials were investigated. Solid and porous (pore sizes ranging from 50 to 150 micrometer) films of pure polymer scaffolds were cut into pieces (5×5 mm). After cleaning with NaOH and ethanol, they were dip-coated with native and nitrocarboxylic acids. Dip-coated pieces were suspended in a tube filled with argon and heated at 60° C. for 24 hours in the dark. Film pieces with and without coating were placed in a borosilicate glass tube that allowed fixation of two margins of the film pieces at the wall of the glass tube, thus, enabling a upright standing position in the center of the tube. Tubes were filled with various solutions for 12 hours. Solutions consisted of the following: 0.9% saline; 2% bovine albumin; 2% bovine albumin with addition of either fibronectin or laminin; and bovine serum. At the end of the exposure time, tubes were gently washed twice with 0.9% saline solution. One set of films was analyzed for protein absorption using a specific antibody staining method. An identically prepared set of films was further processed for cell cultures. A suspension containing preincubated fibroblasts in 1% FCS was added to the film-containing tubes. The tubes were tilted and adjusted in such a position so that the films were in a vertical orientation within the suspension. Tubes were place on a motorized see-saw, which resulted in a continuous back and forth movement of the suspension in the longitudinal direction of the tubes. Tubes had two openings at the upper half (of the tilt tubes) that allowed free exchange of the atmosphere above the solution with the surrounding atmosphere. Sets were incubated at standard conditions for 24, 48, and 96 hours, respectively. Thereafter, films were carefully removed and rinsed with 0.9% saline solution. The cellularity and the shape of the cells on both sides of the films were evaluated after staining (Gimsa) using a reflected light microscope.

### Results:

1. All native films exhibited relatively homogeneous layers of albumin with the exception of the control (saline) films. The protein layers were denser when fibronectin or laminin was present in the solution or serum was used. Specific staining for complement factors revealed that these were present on the surface. Films coated with native fatty acids exhibited lower amounts of albumin, while films coated with nitro fatty acids had the lowest amount of albumin. This was also true when films were exposed to serum. Fibronectin and laminin were densely distributed on the native films, while the amount was less on films coated with native fatty acids; however, this was not observed on films coated with nitrated fatty acids. In addition, the amount of complement factors resembled the amount of albumin on the surfaces.
1. In cell studies, native films exposed to saline solution for 24 hours resulted in only the occasional fibroblasts being attached to the upper surface. After 36 hours there were a few fibroblasts attached, and after 92 hours there were cell islands. On the lower surface of the films, cells were present only after 96 hours. Films coated with native fatty acids and exposed to saline solution exhibited large islands of fibroblasts after 24 hours which confluenced partially after 36 hours, while there was more or less complete attachment on the upper surface after 92 hours. On the lower surface, only slightly fewer cells compared to the upper surface were counted. Films coated with nitrated fatty acids were almost entirely covered with fibroblasts at 24 hours and completely covered after 36 hours on both surfaces. Native films exposed to albumin or serum exhibited an increased rate of attached fibroblasts with complete coverage after 36 hours, when fibronectin or laminin was added, or after 92 hours without exposure to them. Films coated with native fatty acid and exposed to albumin or serum exhibited higher cellularity which was comparable to that of uncoated films. The cellularity on films coated with nitrated fatty acids after exposure to albumin or serum was similar to the cellularity observed in films exposed to saline solution, but slightly lower than the cellularity of native films. Pretreatment with laminin or fibronectin enhanced cell count on non-coated films and to a lesser extent on films coated with native fatty acids, but not on films coated with nitrated fatty acids. For all nitrated fatty acids that were used the results were mostly inside the same range. Relative differences can be represented as follows:

Nitro fatty acids tested

| 1 | 2 | 3 | 4 | 5 | 6 | 7 | 8 | 9 | 10 | 11 | 12 | 13 | 14 | 15 | 16 | 17 | 18 | 19 | 20 | 21 | 22 |
|---|---|---|---|---|---|---|---|---|---|---|---|---|---|---|---|---|---|---|---|---|---|
| ++ | ++ | ++ | + | ++ | ++ | ++ | + | + | +++ | + | + | + | + | ++ | ++ | + | + | + | ND | ND | + |

0 = not superior to native FA (control); + = Superior to native FA; ++ superior to both; +++ outstanding effect; ND = not determined

Cell shape differed considerably between the various coatings. While on native films and on films coated with native fatty acids exposed to albumin or serum, cells were flattened and had a longitudinal or polygonal (dendritic) shape, the cells attached to surfaces containing native fatty acids without pretreatment or attached to surfaces coated with nitrated fatty acids had a rounded shape with only occasional extensions and showed an incomplete attachment to the films.
2. Concentrations of TGF-β in the culture medium were measured. As a rule the concentrations of TGF-β in experiments with native films and films covered with native fatty acids being pretreated with albumin or serum correlated with the measured cell counts. However, this was not the case for investigations performed with films covered with nitrated fatty acids, where considerably lower concentrations of TGF-β were determined. The following relationship with respect to the shape of the fibroblasts was observed: TGF-β values were lower in the presence of cells with rounded shape.
   Conclusions: Dip-coating of polymeric scaffolds with nitrated fatty acids reduces adsorption of extracellular matrix proteins. Attachment of fibroblasts on scaffolds coated with nitrated fatty acids seems to be independent of the matrix protein adsorption onto the artificial surface. This might be the reason for the lower production of TGF-β, thus, indicating reduced stimulation of matrix protein production.

### Example 2

Investigation to Evaluate Effects of Nitrocarbox Ic Acids on Adhesion, Propagation and Growth of Endothelial Cells

Polystyrene scaffolds were prepared and pretreated in the same manner as performed in example 1 by using the same nitrocarboxylic acids as listed in table 1. The film samples were placed in a culture dish containing a gel matrix, in which human umbellical endothelial cells (HUVEC) had been allowed to grow to confluence. The culture medium consisted of 5% FCS, which was replaced every 5 days. Cultivation was performed according to standard conditions. Films were evaluated at days 3, 7, and 14 after careful extraction from the culture dishes, rinsing with saline solution, and staining with methylene blue. Using a reflected light microscope, the films were immediately examined to evaluate the following: Propagation of cells to the film center, cell confluidity, multilayer formation, and cell shape.

Cell propagation was fastest on uncoated films leading to almost complete confluence at day 3. Cell propagation was slower on films coated with native fatty acids with a completion of confluence at day 7. On films coated with nitrated fatty acids, propagation was much slower without completion of cell confluence at day 14. Multilayer formation was observed on uncoated films at day 3 which dynamically progressed with time. In contrast multilayer formation was not observed on films coated with native fatty acids at day 3 and was only present at the outer portion at day 7 and 14. On films coated with nitrated fatty acids, multilayer formation was not observed at any time. The shape of cells propagating on native films was flat and polygonal, while it was less polygonal in cells when propagating on films coated with native fatty acids. On films coated with nitrated fatty acids, cells had a rounded appearance throughout the whole time course and seemed to have less contact area with the film surface. For all nitrated fatty acids that were used the results were mostly inside the same range. Relative differences can be represented as follows:

Nitro fatty acids tested

| 1 | 2 | 3 | 4 | 5 | 6 | 7 | 8 | 9 | 10 | 11 | 12 | 13 | 14 | 15 | 16 | 17 | 18 | 19 | 20 | 21 | 22 |
|---|---|---|---|---|---|---|---|---|---|---|---|---|---|---|---|---|---|---|---|---|---|
| ++ | +++ | ++ | + | ++ | ++ | + | + | + | ++ | + | + | ++ | + | + | + | 0 | + | + | 0 | 0 | ++ |

0 = not superior to native FA (control); + = Superior to native FA; ++ superior to both; +++ outstanding effect; ND = not determined

### Example 3

### Investigation to Evaluate the Effects of Native and Nitro-Fatty Acids on Mechanical Alteration of Fibroblasts

In order to simulate the effects of chronic shear forces on a healing wound, an in vitro model was established. A flat balloon was placed on the bottom of a Petri dish. A silicone sheet was placed above and sealed with the side of the Petri dish. Then a 3 mm agar layer was casted on top of the sheet. Commercially available polypropylene meshes for hernia repair (microporous mesh, a low-weight and macroporous mesh with absorbable polyglactin filaments, and a heavy-weight and microporous mesh) were placed on the agar plates and fixed at 4 points at the side of the Petri dish. This setting enabled stretching of the meshes by filling the balloons with air, which was performed at 10-second intervals using an automated pumping device. The model can be used to evaluate the effect of three-dimensional (3D) shear forces on cell growth during cell cultivation. Preincubated suspensions of fibroblasts (1.5×10(5) cells) in a culture medium (10% FCS) were added to the culture dishes and allowed to grow for 48 hours. Cyclic stretching was started at day 3. Histological analysis was performed from the central portions of the meshes at day 7, at day 21, and after 8 weeks. The scaffolds were detached from the culture plate and carefully rinsed. Then, they were cut into pieces, casted, and further processed for standard histology and immunohistology. Care was taken to achieve a cutting plane that was vertical to the surface plane of the meshes. Histological analysis evaluated the cellularity, the content of extra cellular matrix (ECM), and the magnitude of protein synthesis. Meshes were dip-coated with native and nitro fatty acids or left blank in the same manner as in example 1. The uncoated meshes served as controls.

Results: In uncoated meshes, the number of cells present within the meshes was low (<25% of area) at day 7 and increased markedly after 21 days (50-75% of area). A complete cell matrix texture was observed at 8 weeks. Meshes coated with native fatty acids exhibited a higher cellularity than that observed in control meshes with cells predominantly aligned along the filaments (25-50% of area) at day 7. Cellularity was increased at day 21 (50-75% of area) and was complete after 8 weeks. In experiments with nitrated fatty acid coated meshes, cellularity was similar to experiments with native fatty acids; however, fibroblasts were more often associated with mesh filaments at day 7. Cellularity was less compared to native fatty acids at day 21 and after 8 weeks (75-100%). The shape of the cells differed markedly. In uncoated mesh experiments, fibroblasts had a dendritic shape with lamellar extensions throughout the duration of the investigation, while the shape of the fibroblasts was more rounded in coated meshes, being more pronounced in meshes coated with nitrated fatty acids. During follow-up, they developed a fusiform appearance. Fewer interconnections were observed between the fibroblasts in meshes covered with nitrated fatty acids compared with meshes coated with native fatty acid or without any coating.

Quantification of actinmyosin filaments can be summarized as follows: Expression of actinmyosin filaments within fibroblasts were the same in all samples at day 7. The density of actinmyosin filaments increased up to the end of follow-up in fibroblasts in uncoated meshes and in meshes coated with native fatty acids. However, fibroblasts in meshes coated with nitrated fatty acids had a lower density of actinmyosin filaments than those in uncoated meshes, and there was only a marginal increase in the density of actinmyosin filaments between day 21 and the end of follow-up (FIG. 7). Quantification of protein synthesis revealed increased protein synthesis in fibroblasts in uncoated meshes during follow-up. This finding was paralleled but was at a lower amount for the analysis of fibroblasts in meshes coated with native fatty acids. The protein synthesis of fibroblasts within meshes coated with nitrated fatty acids was lower than that measured in meshes coated with native fatty acids and significantly lower than in non-coated meshes.

Nitro fatty acids tested

| 1 | 2 | 3 | 4 | 5 | 6 | 7 | 8 | 9 | 10 | 11 | 12 | 13 | 14 | 15 | 16 | 17 | 18 | 19 | 20 | 21 | 22 |
|---|---|---|---|---|---|---|---|---|---|---|---|---|---|---|---|---|---|---|---|---|---|
| +++ | +++ | ++ | + | ++ | ++ | + | + | + | ++ | + | + | + | 0 | + | ++ | + | + | ++ | ND | + | ++ |

0 = not superior to native FA (control); + = Superior to native FA; ++ superior to both; +++ outstanding effect; ND = not determined

Conclusions: In a model of chronic tensile stress, coating of polymeric meshes with fatty acids reduces the proliferation of fibroblasts and the production of extracellular matrix proteins. In meshes coated with nitrated fatty acids, however, cell proliferation and matrix production is further reduced and the number of cells that seem to be in a resting phase after 8 weeks is higher compared to non-coated meshes or those coated with native fatty acids.

### Example 4

### Investigation to Evaluate Toxin Response of Cells Incubated with Nitrocarboxylic Acids

In order to determine membrane stabilizing and anticytotoxic properties of nitrated fatty acids, an in vitro model utilizing canine cutaneous mastocytoma cells was chosen. Cells were cultured according to standard procedure. Cytotoxic effects were quantified by measuring not only by Ca²⁺ influx, histamine, and TNFα release, but also by using the MTT assay. Cell suspensions were incubated with 0.9% saline, one of the nitrocarboxylic acids as listed in table 1 or the respective native fatty acid as to achieve a final concentration of 10 and 100 μmol which was given to the medium 15 min before toxin application.

Mastoparan suspended in a buffer solution was added to the preincubated mast cell suspensions to achieve a final concentration of 10 or 30 μmol, respectively. Measurements were performed after 1 hour incubation time. Mastoparan resulted in a dose-dependent Ca²⁺ influx, release of histamine, and induction of apoptosis after preincubation with saline and native fatty acids. Preincubation of mast cells with nitrated fatty acids reduced the effects on Ca²⁺ influx, histamine release, and apoptosis induction in a dose-dependent manner, with an almost complete absence of apoptosis at a concentration of 100 μmol.

Streptolysin O was given to preincubated suspensions to achieve a final concentration of 500 ng/ml. Measurements were performed after 2 hours. Releases of histamine and TNFα in the suspensions were measured. After saline preincubation, a significant release of histamine and TNFα was observed. The release of both was nonsignificantly reduced by preincubation with native fatty acids at high concentrations (100 μmol). Preincubation with nitrated fatty acids resulted in a dose-dependent reduction in the release of histamine, which was significantly lower at high concentrations (100 μmol).

Cell plates were exposed to a single dose (250 mJ/cm2) of UVB using a lamp (Saalmann, Herford, Germany) emitting most of its energy within the UVB range (295 to 315 nm). Tryptase levels in the supernatants were determined after 30 minutes, and concentrations of TNFα after 60 minutes. After preincubation with saline, there was a marked increase of tryptase. Preincubation with native fatty acids led to a dose-dependent reduction of tryptase release, which was significant at the lower irradiation dose but not after exposure to a high irradiation dose. In contrast, preincubation with nitrated fatty acids at both concentrations resulted in a significant reduction in tryptase release compared to saline at the lower irradiation dose, and a significant reduction after preincubation with the high concentration of the nitrated fatty acids in tryptase release when cells were exposed to the high irradiation dose. The concentrations of TNFα increased significantly in samples pretreated with saline. After preincubation with native and nitrated fatty acids, a reduction of TNFα increase was found that paralleled the reductions found for the tryptase measurements, which were significantly lower after preincubation with nitrated fatty acids compared to native fatty acids when cells were exposed to the high irradiation dose. For all nitrated fatty acids that were used the results were mostly inside the same range. Relative differences can be represented as follows:

Nitro fatty acids tested

| 1 | 2 | 3 | 4 | 5 | 6 | 7 | 8 | 9 | 10 | 11 | 12 | 13 | 14 | 15 | 16 | 17 | 18 | 19 | 20 | 21 | 22 |
|---|---|---|---|---|---|---|---|---|---|---|---|---|---|---|---|---|---|---|---|---|---|
| +++ | ++ | +++ | + | + | ++ | ++ | + | ++ | +++ | + | ++ | ND | ND | ++ | ++ | + | 0 | 0 | ND | ND | ++ |

0 = not superior to native FA (control); + = Superior to native FA; ++ superior to both; +++ outstanding effect; ND = not determined

Conclusions: Preincubation of mast cells with nitrated fatty acids reduces membrane destabilization by toxins. Since degranulation of mast cells by mastoparan is known to be membrane protein-mediated, nitrated fatty acids act as modifier of transmembrane signal transduction probably by changing physical membrane properties. This conclusion is supported by the results obtained with Streptolysin O, which exerts its toxic effects by interacting with membrane cholesterol. This interaction is probably inhibited by the nitrated fatty acids. The same is likely to be the reason for the reduced cytotoxic effects of irradiation energy. Thus, the results imply that preincubation with nitrated fatty acids reduce the action of toxins by reducing membrane permeability and influencing signal membrane transduction pathways.

### Example 5

### Investigation to Evaluate the Effects of Native and Nitro Fatty Acids on the Signal Transduction of Receptors of the TRP-Protein Family

In order to determine the effect of nitrated fatty acids on receptor signal transduction, an in vitro model was used. Retinal slices from cadaver mice were prepared as described by Snellman and Nawy (Snellman J. Nawy S (2004). cGMP-dependent kinase regulates response sensitivity of the mouse on bipolar cell. J Neurosci 24:6621-6628). Whole retinas were isolated and cut into 100 μm slices using a tissue slicer and than transferred to the recording chamber. The chamber was continuously perfused with Ames media and oxygenated. Picrotoxin (100 μM), strychnine (10 μM), and TPMPA (50 μM) were added. Each nitro fatty acid was added in two experiments to achieve a final concentration of 10 μmol, and in another two experiments to achieve a final concentration of 50 μmol. In two experiments, the corresponding native fatty acid was added to achieve a final concentration of 10 and 50 μmol, and in two experiments saline solution was added which served as controls. Currents were measured via tissue electrodes and monitored throughout the investigation. After incubating the solution for 2 hours, the TRPV-1 agonist capsaicin was added to achieve a final concentration of 10 μmol. Experiments were performed using either 10 mmol Hepes or 10 mmol Mes adjusting the pH of the solution to 6.4 or 4.4, respectively. Furthermore, experiments were performed without or with preincubation (5 minutes) of the TRPV-1 antagonist capsazepine. The solution temperature was held constant at 35° C.

Both pH reduction and capsaicin application induced a current in specimens preincubated in saline. The capsaicin effect was inhibited when capsazepine was preincubated. Preincubation with the low concentration of native acid had no effect on the current response to capsaicin and acid; however, preincubation with the high concentration reduced the capsaicin-induced current slightly but not the current induced in an acidic environment. Preincubation with the low concentration of nitro fatty acids had a modest effect on the current response to capsaicin. However, preincubation with the high concentration of nitro fatty acids almost completely inhibited current response to capsaicin and led to a reduced current response to acid (60% compared to saline).

Conclusions: The TRPV receptors serve as nociceptors in the peripheral nerve system with a predominance of the subtype TRPV-1. Its stimulation leads to pain sensation. Nitrated fatty acids reduce the agonist capacity at the receptor level, probably by inhibiting membrane protein-mediated membrane signal transduction.

Nitro fatty acids tested

| 1 | 2 | 3 | 4 | 5 | 6 | 7 | 8 | 9 | 10 | 11 | 12 | 13 | 14 | 15 | 16 | 17 | 18 | 19 | 20 | 21 | 22 |
|---|---|---|---|---|---|---|---|---|---|---|---|---|---|---|---|---|---|---|---|---|---|
| ++ | ++ | ++ | ND | ND | + | ND | ND | ND | ++ | ND | ND | ND | ND | ND | ND | ND | ND | ND | ND | ND | + |

0 = not superior to native FA (control); + = Superior to native FA; ++ superior to both; +++ outstanding effect; ND = not determined

### Example 6

### Investigation to Evaluate the Effects of Native and Nitro Fatty Acids on Membrane Protein Mediated Cytotoxicity in Epithelial Cells

Human epithelial lung cells (A549) were cultured and transferred to an isotonic medium. Cell suspensions were incubated with saline solution, native fatty acid (10 and 50 μmol), or nitro fatty acid (10 and 50 μmol) for 2 hours. Sodium fluoride (NaF) was added to achieve concentrations between 1 and 8 mmol. Cells were separated and washed after 24 hours of exposure. The MTT assay was used to evaluate apoptosis rates. NaF induced apoptosis in a dose-dependent manner in the control group. Native fatty acids reduced apoptosis moderately when incubated with the high concentration but not at the lower concentration. Incubation with nitro fatty acids at the low concentration reduced apoptosis to a similar extent as the native fatty acids at the high concentration; however, preincubation of nitro fatty acids at the high concentration almost completely prevented apoptosis.

Conclusions: NaF-induced apoptosis has been demonstrated to be membrane protein-linked in human epithelial lung cell lines. Therefore, the reduction in cytotoxicity of NaF by incubating cells with nitro fatty acid is likely to be attributable to the modifying effect on the signal transduction of transmembrane proteins that can be induced by a change of membrane fluidity induced by nitro fatty acids.

Nitro fatty acids tested

| 1 | 2 | 3 | 4 | 5 | 6 | 7 | 8 | 9 | 10 | 11 | 12 | 13 | 14 | 15 | 16 | 17 | 18 | 19 | 20 | 21 | 22 |
|---|---|---|---|---|---|---|---|---|---|---|---|---|---|---|---|---|---|---|---|---|---|
| ++ | ++ | + | ND | ND | + | ND | ND | ND | + | ND | ND | ND | ND | ND | ND | ND | ND | ND | ND | ND | ++ |

0 = not superior to native FA (control); + = Superior to native FA; ++ superior to both; +++ outstanding effect; ND = not determined

### Example 7

### Investigation to Evaluate the Effects of Nitrocarboxylic Acids on Adhesion of Serum Proteins and Monoblast Activation Induced by Implant Material

In order to determine the effect of coating implant materials with nitro fatty acids to reduce adsorption of adhesion molecules and monocyte activation, sterile silicone sheets were out in small pieces and coated with respective nirocarboxylic acid and the corresponding native fatty acids by dip coating. Uncoated silicone pieces served as controls. For each fatty acid two sets of silicone pieces were bathed in freshly drawn human serum for 1 hour at 37° C., and another two sets were bathed in saline solutions. One set of both coated and uncoated pieces was analyzed immediately for adhesion proteins and the other set was placed in 96-well plates after rinsing the silicone pieces. Human peripheral blood mononuclear cells (PBMCs), isolated from three healthy subjects, were added to each well. Wells were incubated for 3 days under standard conditions. Culture supernatants were assayed for IL-1beta, IL-6, IL-8, and chemoattractant protein 1 (MCP-1) levels at the start and end of experiments.

Before analyzing the silicone pieces for protein adsorption, they were bathed in saline for 5 minutes. Thereafter, one side was rinsed with saline as performed for both sides in the set used for culturing. There was marked adsorption of fibrinogen and monocyte anchoring complex C5b-9 on the uncoated silicone. Coating with a corresponding non-nitrated fatty acid resulted in a slight reduction in the amounts of protein detected, while coating with the nitro fatty acid acids almost completely abolished protein adsorption. No protein was found on the surface of the nitro fatty acid-coated sample that was rinsed, thus, indicating the weak adherence of serum proteins.

Results: Serum exposure of uncoated samples resulted in a substantial increase of IL-8 and MCP-1, and a marked increase of IL 1-beta and IL 6 by cultivated PBMCs. Coating of the silicone sheets with native fatty acid resulted in a moderate reduction of all cytokine production in samples without serum conditioning. A marked reduction of IL-1beta, IL, and MCP-1 was found in samples conditioned with serum compared to noncoated samples; however. IL-8 was only slightly reduced. By contrast, cytokines could not be detected in cultures of samples coated with nitro fatty acids when preconditioned with saline and were found to be at the lower detection limit when samples were incubated with serum, however, IL-8 could not be detected.

Nitro fatty acids tested

| 1 | 2 | 3 | 4 | 5 | 6 | 7 | 8 | 9 | 10 | 11 | 12 | 13 | 14 | 15 | 16 | 17 | 18 | 19 | 20 | 21 | 22 |
|---|---|---|---|---|---|---|---|---|---|---|---|---|---|---|---|---|---|---|---|---|---|
| +++ | ++ | ++ | + | + | + | 0 | + | + | ++ | + | 0 | + | + | + | 0 | + | ++ | + | + | 0 | + |

0 = not superior to native FA (control); + = Superior to native FA; ++ superior to both; +++ outstanding effect; ND = not determined

Conclusions: Silicone like other materials used for implants rapidly adsorbs serum proteins, e.g., fibrinogen and complement, the latter forming the monocyte anchoring complex C5b-9. Serum protein adsorption leads to a marked release of monocyte-derived cytokines. While native fatty acids only had a minor effect on protein adsorption and the consecutive release of cytokines, nitrated fatty acid leads to a marked reduction of protein absorbance and eases the removal of the monocyte anchoring complex C5b-9. The low protein adhesion explains the absence of relevant monocyte activation and cytokine production.

### Example 8

### Investigation to Evaluate the Effects of Nitrocarboxylic Acids on Adhesion and Proferation of Monocytes and Fibroblasts on Surgical Suture Material

In order to determine immunoreactions to foreign material and its consequence on fibrogenesis, co-cultures of fibroblasts and monocytes were used. Commercially available suture materials (propylene, polyamide, and silk) were dip-coated with corresponding native and nitrated fatty acids. Untreated suture material served as control. Treated and untreated suture materials were out in small pieces and placed in a 96-well plate.

Murine macrophage-like cells RAW 264.7 and murine L929 fibroblasts were cultured to a population density of 5×10(5) each. Cell suspensions were merged achieving a cellularity of approximately 2.5×10(5) cell/ml of each cell population which were added to the wells. The suture material samples were fully covered by the suspension. Wells were continuously and gently shaken throughout the incubation period.

Supernatants were collected for assays after 24 and 48 hours and analyzed for profibrotic cytokines IL-13, IL-4, and IL-6, TGF-β1, collagen I.

Results: In uncoated suture materials, there was a marked increase in all cytokines and collagen I. In supernatants of suture materials coated with a native fatty acid, a significant reduction of IL-13 and TGF-β1 was found after 24 hours compared to uncoated suture material, which was not significant any more at 48 ours. The other cytokines and the collagen content were lower compared to values found in uncoated suture material. For supernatants of samples coated with nitro fatty acids, cytokines and collagen content was significantly lower compared to values obtained in suture materials coated with native fatty acids.
Conclusions: Conventional surgical suture material causes a rapid rise in production of cytokines that stimulate fibrogenesis when exposed to cultured monocytes. Accordingly, co-cultured fibroblasts react rapidly by production of extracellular matrix components. Cytokine production can be reduced by coating suture materials with native fatty acid and significantly reduced when nitrated fatty acids are used for material coating.

Nitro fatty acids tested

| 1 | 2 | 3 | 4 | 5 | 6 | 7 | 8 | 9 | 10 | 11 | 12 | 13 | 14 | 15 | 16 | 17 | 18 | 19 | 20 | 21 | 22 |
|---|---|---|---|---|---|---|---|---|---|---|---|---|---|---|---|---|---|---|---|---|---|
| ++ | +++ | ++ | + | + | + | + | + | + | +++ | 0 | + | + | + | + | + | + | 0 | + | 0 | + | + |

0 = not superior to native FA (control); + = Superior to native FA; ++ superior to both; +++ outstanding effect; ND = not determined

### Example 9

### Investigation of the Efficacy of Nitrocarboxylic Acids in a Model for Fibrosis Induction

Cornea injury may ultimately lead to a scar by way of corneal fibrosis, which is characterized by the presence of myofibroblasts and improper deposition of extracellular matrix components (ECM). An established in vitro model to study the healing response to trauma of corneal stroma was used. The in vitro 3-dimensional (3D) model of a corneal stroma was produced by human corneal fibroblasts stimulated with stable vitamin C which mimics corneal development. TGF-β1 was added to the medium over 7 days. As compared to the control group, the 3D cell-size increased significantly, cells became long and flat, numerous filamentous cells were seen, collagen levels increased and long collagen fibrils could be seen, as present in corneal fibrosis. Addition of nitro-fatty acids to TGF-β1 exposition for 10, 30 and 60 minutes significantly suppressed fibrosis as compared to 0.9% saline, and the respective native fatty acids in similar concentrations. There were no morphological changes of the myofibroblasts when treated with nitro fatty acids. Deposition of ECM paralleled the development of fibrosis and was significantly reduced by the nitro fatty acids as compared to the control groups.

Nitro fatty acids tested

| 1 | 2 | 3 | 4 | 5 | 6 | 7 | 8 | 9 | 10 | 11 | 12 | 13 | 14 | 15 | 16 | 17 | 18 | 19 | 20 | 21 | 22 |
|---|---|---|---|---|---|---|---|---|---|---|---|---|---|---|---|---|---|---|---|---|---|
| ++ | ++ | + | ND | ND | ++ | ND | + | ND | ++ | + | ND | ND | + | + | ND | ND | ND | ND | ND | ND | ++ |

0 = not superior to native FA (control); + = Superior to native FA; ++ superior to both; +++ outstanding effect; ND = not determined

### Example 10

### Investigation of the Efficacy of 6-Nitro-Cis-6-Octadecenoic Acid (Nitro-Petroselinic Acid) and 11-Nitro-Cis-5,8,11,14-Eicosatetraenoic Acid (Nitro-Arachidonic Acid) in a Foreign Body Model

Organisms react to the contact with a not completely biocompatible surface with a stereotypic chain of reactions. This is preceded by aggregation of plasmaproteins which initiate adhesion of monocytes. As a response to incompatibility, structural changes and fusion of those monocytes to form giant cells occur. The formation of giant cells is a key component in the development of a foreign body reaction. It was found that interleukin-4 (IL-4) produced by activated macrophages is essential for the formation of giant cells. Predictability of foreign body reaction has been validated in an in-vitro model by monitoring fusion of macrophages in response to IL-4 exposition. Using this model a polymeric coating of a stainless steel support material containing variable amounts of nitro fatty acids or native fatty acids or just coated with the sole polymer were exposed to plasma with various concentrations of vitronectin. Compared to a coating with polymer alone, the fusion of macrophages was completely inhibited by the nitro-fatty acids at the used concentrations, while the non-nitrated fatty acids showed a minor inhibition. Accordingly, the concentration of measured IL-4 rose insignificantly in the supernatant culture media of cell cultures which were exposed to nitro-fatty acids while a significant rise was observed in cultures with polymer alone or native fatty acid coating.

### Nitro fatty acids tested

0=not superior to native FA (control); +=Superior to native FA; ++superior to both; +++outstanding effect; ND =not determined

nitro-petroselinic acid: +nitro-arachidonic acid: ++

These results indicate that the foreign body response to the exposure of artificial material coated with a polymeric surface containing nitro-fatty acids is significantly reduced.

### Example 11

### Investigation of the Efficacy of Nitro Fatty Acids to Suppress TGF-β1-Inducible Formation of Extracellular Matrix in Cardiomyocytes

Characterizing features of the fibrous remodelling in the heart are accentuated expression and deposition of extracellular matrix proteins (ECM). This has been attributed to increased mechanical forces via autocrine release of transforming growth factor-beta (TGF-beta). It has been shown in isolated single cardiomyocytes that stimulation with TGF-beta results in extracellular matrix protein deposition, suggesting cardiomyocytes as a primary source for the fibrotic changes seen in ventricular hypertrophy. An established cell culture model was used to investigate the effect of shear stress on cardiomyocytes. Cardiomyocytes were cultured and transferred to a matrigel substrate. Plates with confluenced cells were placed into a shear force injury device (SFID). The SFID design is based on a cone-and-plate construction which is a well-defined rheological model in which a homogenously distributed laminar flow over the surface of the cells is generated by a rotating cone. The conical surface is positioned above a stationary flat plate and the fluid medium between these two surfaces is set in motion by rotating the cone to create a uniform level of fluid shear stress throughout the entire surface of the cells cultured on the coverslips.

A peak shear stress of 100 dyn/cm² could be applied without significant cell detachment enabling a maximum injury severity of 46%. Cells were exposed to FCS 1% without or with nitro-fatty acid, and the corresponding native fatty acid in a dosage range between 10 μmol and 100 μmol, 10 seconds before shear force application. Shear force peaks up to 100 dyn/cm² with a duration of 30 msec each with a repetition frequency of 60/minute were applied for 5, 10, 30 and 60 minutes. Thereafter the cell plates were washed and placed in FCS 1% for 24 hrs. The supernatant of the shear force investigation as well as that of the following culture phase was analysed. It could be shown that TGF-beta and ECM proteins (collagen I, fibronectin, laminin, elastin) increased after treatment in the control groups. Nitro-fatty acids reduced TGF-beta and ECM protein concentration/amount significantly in a dose-dependent manner with a maximum suppression reached at a concentration of 50 μmol/l.

Nitro fatty acids tested

| 1 | 2 | 3 | 4 | 5 | 6 | 7 | 8 | 9 | 10 | 11 | 12 | 13 | 14 | 15 | 16 | 17 | 18 | 19 | 20 | 21 | 22 |
|---|---|---|---|---|---|---|---|---|---|---|---|---|---|---|---|---|---|---|---|---|---|
| ++ | ++ | ++ | + | + | +++ | + | + | + | ++ | + | ND | ND | + | + | ND | ND | ND | ND | + | ++ | ++ |

0 = not superior to native FA (control); + = Superior to native FA; ++ superior to both; +++ outstanding effect; ND = not determined

### Example 12

### Biocompatible Coating of a Saline Breast Implant with Nitro-Oleic Acid Under Adding of a Catalyst and a Synthetic Polymer, Especially Polyvinylpyrrolidone

Non-expanded stents of poly(tetrafluoroethylene) are removed from fat in the ultrasonic bath for 15 minutes with acetone and ethanol and dried at 40° C. in the drying oven. Subsequently, the breast implant is washed with demineralized water over night. About 10 mg of KMnO₄ are dissolved in 500 μl of water and as much as possible PVP is added. The mixture is spread laminarly on a polypropylene substrate and allowed to dry at room temperature over night. From this brittle mixture 2.5 mg are dissolved in 1 ml of chloroform and the resulting solution is sprayed after adding of 10.5 μl of nitro-oleic acid with an airbrush spraying pistol (EVOLUTION from Harder & Steenbeck) from a distance of 6 cm on a rotating 18 mm LVM stainless steel stent. Afterwards the coated breast implant was stored for 24 h at 40° C.

### Example 13

### Complete Coating of a Mesh with Nitro-Linoleic Acid by Means of Pipetting Method

The mesh is spread in a horizontal position and thus mounted onto a rotatable axis. Thus, step by step the ethanol-dissolved nitro-linoleic acid is applied along the longitudinal axis row by row with a teflon canula as extension of a syringe tip until a continuous nitro-linoleic acid layer can be observed. Then the mesh is dried.

Preferably an adjuvant which facilitates the permeability of the agent into the cells is added to the agent solution. For example, 150 mg of nitro-linoleic acid, 4.5 ml of acetone, 100 μl of iodopromide and 450 μl of ethanol are mixed.

### Example 14

### Complete Coating of a Silicone Breast Implant with Nitro-Arachidonic Acid by Means of the Vapor Deposition Method

The silicone breast implant is placed on a table inside the vacuum chamber. Nitro-arachidonic acid dissolved in dimethyl ether is filled into a cavity inside the vacuum chamber. A vacuum of 3 Pa is generated inside the vacuum chamber. Ultrasound (10 MHz, 12 MPa sound pressure, 5 min) is applied to the cavity containing the coating solution. Then such dispersed coating solution is released into the vacuum chamber for depositing on the implant surface. The procedure is repeated six times.

### Example 15

### Investigation to Evaluate the Effects of Surface Coating with Nitrocarboxylic Acids on Fibrogenesis in an In-Vivo Model of Soft Implant Implantation

### Preparation of Silicone Implants:

Silicone bag-gel miniprostheses (POLYTECH Health & Aesthetics GmbH, Dieburg, Germany) having a diameter of 2 cm and a volume of approximately 2 ml were used. Materials and construction were comparable to regular breast implants and consist of a soft silicone rubber shell containing a viscous silicone gel filling. The experimentally used implant models had two small tags which allowed the implants to be suspended during the coating procedures.

Each implant was treated in the following manner: cleaning by sonication for 2 min each in the following sequence of solvents: acetone, toluene, acetone, ethanol, and water. Implants were exposed to Piranha solution for 60 min and rinsed with deionized water. They were then immersed in a 20% aqueous solution of ammonium fluoride for 45 min to obtain a hydrogen-passivated Si surface. The ammonium fluoride solution was sparged with nitrogen for 15 min to remove dissolved oxygen. Prepared implants were transferred to a glass chamber filled with an inert atmosphere where they were suspended so that no part of their surface came in contact with the container. The container was filled with a 1-hexadecene solution and heated at 150° C. at a pressure of 2 Torr for 120 min. Prepared implants were cleaned in the following sequence of solvents: acetone, ethanol, and water. Formation of self assembled monolayer was controlled by measuring surface hydrophobicity; the contact angle was about 105°. Dry implants suspended in the container were then bathed in an ethanolic solution of oleic acid or nitro-oleic acid at a temperature of 40° C. for 120 min. Thereafter, the solution was allowed to run out slowly through an outlet at the bottom of the container. The emptying container was filled with inert gas in which the samples were maintained for 24 hours. Then samples were bathed three times in an ethanolic solution, followed by a final rinsing with sterile water. After drying, the container with the prepared implant was sterilized using ethylene oxide and stored at 20° C.

For in vivo testing, 24 uncoated and 24 coated implants were investigated in 24 female Sprague-Dawley rats (190-230 g). In the anesthetized animals, bilateral dorsal pockets in the subcutaneum were created by blunt dissection through paired paravertebral incisions. Each animal received one coated implant and one control implant on opposite sides, alternating sides on successive animals. Animals were housed and fed according to institutional standards for 120 days. Animals were sacrificed with chloroform. The implants and the adhering tissue were extracted by en bloc resection. The implants were cannulated and the liquid silicone gel was replaced by paraffin. Thereafter, the complete tissue block was prepared by standard means of histology, and stained with H&E or Masson's trichrome.

Results: In uncoated implants a marked fibrotic capsule was observed in all animals. Coating with native fatty acids reduced the thickness of the fibrotic capsule to a variable extent. However, in implants coated with nitrated fatty acid, a fibrotic capsule was absent. There were only small areas of connective tissue; therefore, the amount of extracellular matrix was significantly reduced compared to uncoated silicone implants or those coated with native fatty acid coating. Furthermore, foreign body formation was not observed after coating with nitrated fatty acid but was found after coating with native fatty acid and in uncoated implants.

Nitro fatty acids tested

| 1 | 2 | 3 | 4 | 5 | 6 | 7 | 8 | 9 | 10 | 11 | 12 | 13 | 14 | 15 | 16 | 17 | 18 | 19 | 20 | 21 | 22 |
|---|---|---|---|---|---|---|---|---|---|---|---|---|---|---|---|---|---|---|---|---|---|
| ++ | ++ | ++ | ND | ND | ++ | ND | + | + | ++ | + | ND | + | + | + | 0 | ND | ND | ND | ND | + | ++ |

0 = not superior to native FA (control); + = Superior to native FA; ++ superior to both; +++ outstanding effect; ND = not determined

### Example 16

### Investigation to Evaluate the Effects of Nitrocarboxylic Acids on Cold Induced Injury in a Whole Tissue Ex-Vivo Model

To determine whether nitrated fatty acids can be used to preserve cells and tissues from cold or cryoinjury, an in vitro model utilizing murine epicardium was established. The experimental set-up should reduce the possibility of ischemia or reperfusion injury to a minimum, but at the same time allow differentiation of the contribution of ischemia/reperfusion injury-triggered cell death, which is known to occur almost exclusively via apoptosis, whereas cell death induced by cold injury almost exclusively results in necrosis.

The pericardial sac was carefully resected in 24 anesthetized Wistar rats (180-270 g) of either sex. Care was taken to grasp the pericardium with a forceps only at the cutting edges and reduce traumatization of the central portions of the pericardium to a minimum. After resection, the cutting edge and the former apex area of the pericardium were dissected, as to obtain flat tissue strips which were placed immediately in the culture medium (DMEM) containing 10% FCS and antibiotics (penicillin, streptomycin). Tissue samples were cultured in an oxygen atmosphere at 18° C. for 2 days. Thereafter the culture temperature was gradually increased to 37° C. within 5 days. Then the pericardial strips were cut into 4 pieces of equal size. One piece was further cultivated at 37° C. without undergoing the cooling cycles, thus, serving as the control. In each of three investigations, one piece was bathed in a solution (0.9% saline and 1% SDS) containing 200 μmol nitro-fatty acid or contained 200 μmol of the corresponding native fatty acid, for 10 min at a temperature of 18° C. One piece of pericardium was bathed in a solution without fatty acids under otherwise identical conditions. All samples were cooled by continuously decreasing from ambient temperature at a rate of 3° C./min to a minimum temperature of 15° C. After 1 hour, the samples were rewarmed at a rate of 3° C./min by continuously increasing the temperature until a temperature of 18° C., was achieved. Thereafter an identical cooling and rewarming cycle was performed. After the second cooling cycle, the tissue pieces were further cultured in the culture medium for 1 day allowing continuous adaptation to an ambient temperature of 37° C. For analytical preparations the pericardial pieces were out and further processed. A direct TUNEL assay (Roche) was use for quantification of apoptosis, an annexin V/propidium iodide exclusion assay (Roche) was used to quantify the number of necrotic cells, and viability was determined by the WST-8 assay. Furthermore, the LDH release was quantified in the supernatants.

Results: In the control samples not exposed to cold, there was only a slight release of LDH in the supernatant. Correspondingly only isolated cells were found to be propidium iodide negative/annexin positive and TUNEL positive, indicating evolving apoptosis, and a similar number were found to be propidium iodide/annexin positive and TUNEL negative, indicating evolving necrosis of cells. The MTT assay indicated high viability of the sample cells. In contrast, untreated samples exposed to cold exhibited a tremendous increase of LDH and viability was reduced to less than 40% of that of control samples in the MTT assay. Propidium iodide-negative/annexin-positive and TUNEL-positive cells were found only occasionally (<5%); however, this was slightly more than in the control samples. A high proportion (45-60%) of propidium iodide/annexin-positive and TUNEL-negative cells were found, indicating a high number cells undergoing primarily necrosis. In samples exposed to native fatty acids, LDH release was nonsignificantly lower than in the untreated samples. In addition, the viability of cells was reduced to a similar extent compared to untreated samples documented in the MTT assay. A comparable relation and extent of cells undergoing apoptosis or necrosis were observed in the TUNNEL and propidium iodide/annexin labeling. However, in samples incubated in either of the nitrated fatty acids, there was only a small increase of LDH, which corresponded to a high viability that was about 90% of the viability of the control samples. The number of cells undergoing apoptosis was only slightly lower than after pretreatment with the native fatty acids. The number of cells undergoing necrosis, however, was significantly lower in the samples incubated with the nitrated fatty acids (15-20%) compared to samples incubated with the native fatty acids.

Nitro fatty acids tested

| 1 | 2 | 3 | 4 | 5 | 6 | 7 | 8 | 9 | 10 | 11 | 12 | 13 | 14 | 15 | 16 | 17 | 18 | 19 | 20 | 21 | 22 |
|---|---|---|---|---|---|---|---|---|---|---|---|---|---|---|---|---|---|---|---|---|---|
| ++ | ++ | + | ND | ND | ++ | ND | ND | ND | + | ND | ND | ND | ND | ND | ND | ND | ND | ND | ND | + | ++ |

0 = not superior to native FA (control); + = Superior to native FA; ++ superior to both; +++ outstanding effect; ND = not determined

Conclusions: Experiments have shown that under controlled cultivation conditions it is possible to cultivate pericardium without relevant loss of viable cells. Cold-induced cell injury was detected and classified to predominantly induce necrosis, in agreement with other scientific reports. This indicates that cell membrane destructive effects that occur during crystallization and decrystallization, respectively, as reported in the literature, induce necrosis and only to a negligible extent apoptosis. The near absence of apoptotic cells indicates that ischemia or reperfusion injury was prevented by the experimental set-up. Native fatty acids did not have a relevant effect on cold-induced cell injury. On the other hand, nitrated fatty acids were proven to prevent cold-induced cell injury to a large extent in an intact body tissue model. Therefore, nitrated fatty acids are useful to reduce cell injury during cold preservation.

To rule out that cell death has been induced by ischemia or reperfusion (reoxygenation), in one series of experiments, a control sample was incubated with an all-caspase inhibitor (Q-VD-OPH, BioVision, USA) reconstituted in DMSO before the sample was exposed to cold. The rate of cells undergoing apoptosis was within the range found in the cold-exposed control samples, indicating that almost no ischemia/reperfusion injury occurred in the chosen experimental setting.

### Example 17

### Investigation to Evaluate the Effects Nitrocarboxylic Acids on Extra Cellular Matrix Production Response to Endogenous Stimulants in Dermal Fibroblasts

In order to determine whether PPRA gamma stimulation due to nitrated fatty acids plays a role in the inventive inhibition of an aggressive healing pattern to an irritating stimulus, human dermal fibroblasts were investigated. A dominant-negative PPAR gamma mutant (L466A) cell clone was generated by polymerase chain reaction-based site-directed mutagenesis. Presence of PPAR gamma or absence was investigated using a PPAR gamma antibody reaction which was determined using an enhanced chemiluminescence detection system. Furthermore, one cell series was incubated with a selective irreversible PPAR gamma ligand (GW9662, 1 μmol), which effectively blocks PPAR gamma receptors.

Sequential cultivation of human dermal fibroblasts was performed in EMEM containing 5 mM glucose supplemented with 10% FCS under standard cultivation conditions. Cells were allowed to grow to confluence in a 96-well plate. Wild-type fibroblasts and PPAR gamma deficient fibroblasts were investigated by a set-up of 2×10 sample sets comprising: (1) blank control; (2) stimulation control; (3) preincubation with the PPAR gamma antagonist GW9662 (Cayman Chemical); (4) preincubation with the PPAR agonist troglitazone (25 μmol). In 2×4 sample sets, native fatty acids were added to the medium to achieve a final concentration of 10 and 50 μmol, respectively; in another 2×4 sets the nitrated fatty acids were added to the medium in the same concentration, while 2×2 sets served as controls. In 10 of the sets, TGF-β2 was added to the culture medium at a concentration of 25 ng/ml. Cultivation was continued for 48 hours and then processed in order to analyze collagen-1 as measured by enhanced chemiluminescence detection of immuncomplexes.

Results: In blank controls, there was a low concentration of collagen-1 which was not significantly different between PPAR gamma-positive or -negative cells and which was also the case in PPAR-positive cells incubated with the PPAR agonist or antagonist. In both, PPAR-positive and -negative cell cultures stimulation with TGF-β2 resulted in a marked increase of collagen-1 in control experiments and in cells preincubated with the PPAR gamma antagonist. Preincubation with the PPAR agonist reduced collagen-1 concentrations by 35-40% as compared to the control in PPAR-positive but not in PPAR-negative cells. Addition of native fatty acids to unstimulated cell cultures resulted in collagen-1 concentrations that were indistinguishable from identical experiments without addition of fatty acids. In cultures of PPAR-positive and -negative cells that were stimulated with TGF-β2 and preincubated with native fatty acids, there was an increase in collagen-1 concentrations that was 15-25% lower than that measured in control cultures, which was an identical finding in cells preincubated with the PPAR gamma antagonist. In PPAR gamma-positive cells preincubated with the PPAR angonist and native fatty acids, there was a 25-35% reduction of collagen-1 concentrations as compared to that of the controls; this reduction was smaller than the reduction that has been achieved when using the PPAR agonist alone.

Preincubation with nitrated fatty acids lowered the collagen-1 content in unstimulated PPAR gamma-positive and -negative cells as well as in cell cultures which were preincubated with the PPAR gamma agonists or antagonist. Preincubation with nitrated fatty acids in PPAR gamma-positive and -negative cells almost completely inhibited collagen-1 production after TGF-β2 stimulation. Neither preincubation with the PPAR gamma agonist nor the antagonist had a detectable influence on the inhibitory effect of the nitro fatty acids.

Conclusion: Human epidermal fibroblasts produce collagen-1 in response to TGF-(12 stimulation. This stimulatory effect is reduced by a PPAR gamma receptor agonist in PPAR positive but not in PPAR negative cells. The PPAR gamma mediated effect was reduced by preincubation with native fatty acids. In contrast, nitrated fatty acids completely inhibited TGF-β2 stimulated collagen-1 production in PPAR positive and negative cells. Since neither absence of PPAR gamma receptors nor blockage of the PPAR gamma receptors had an influence on the inhibition of TGF-β2 cell signaling obtained by preincubation with nitrated fatty acids, a PPAR gamma-mediated mechanism for this finding can be excluded.

Nitro fatty acids tested

| 1 | 2 | 3 | 4 | 5 | 6 | 7 | 8 | 9 | 10 | 11 | 12 | 13 | 14 | 15 | 16 | 17 | 18 | 19 | 20 | 21 | 22 |
|---|---|---|---|---|---|---|---|---|---|---|---|---|---|---|---|---|---|---|---|---|---|
| +++ | ++ | ++ | + | + | ++ | + | 0 | + | ++ | + | + | + | + | + | 0 | + | + | + | + | ++ | ++ |

0 = not superior to native FA (control); + = Superior to native FA; ++ superior to both; +++ outstanding effect; ND = not determined

### Example 18

### Investigation to Evaluate the Effects Nitrocarboxylic Acids on Fibrogenesis in Response to Traumatic and Thermal Tissue Damage in Dermal Wound Healing In Vivo

In order to determine the effects of nitrated fatty acids on fibrogenesis in response to traumatic and thermal tissue damage, an in vivo rat model was investigated. Paravertebral scalpel incision (two on each side) of approximately 1 cm in length and a depth of 1 mm were made in 12 anesthetized adult rats (150-200 g) of either sex after disinfection. Bleeding was stopped by manual compression. The entire length of the wound margins of one incision at each side were additionally cauterized using a 3-mm ball electrode connected to a DRE ASG-120 electrosurgical generator. Wound margins of one side were then covered with a sterile ethanolic 0.9% saline solution, or with a sterile ethanolic solution of the fatty acids (100 micromol), or with a sterile ethanolic solution of the nitrated fatty acids (100 micromol) using a sterile brush. A 1×10 mm cotton string that was bathed in the ethanolic solution containing 0.9% saline, native fatty acid or nitrated fatty acids was placed upon the incision site that had been closed by manual adaptation. The adaptation result and the cotton strings were fixed by an adhesive film. The animals were housed and fed according to institutional standards. The wound films were carefully removed after 2 weeks. Animals were euthanized after 8 weeks. The skin wounds were harvested, including the epidermis, dermis, and subcutaneous loose tissue with the surrounding normal tissue. The removed tissues were fixed in formalin and then embedded in paraffin. The cutting plane was vertical to the longitudinal axis of the former incisions. Slices (4-6 μm) were stained with H&E and Masson trichrome staining to evaluate the amount and density of collagen.

Results: Histology of incisions treated with 0.9% saline exhibited a typical pattern of scar formation with a mean width of 2.2 mm. In incisions with additional cauterization, there was higher cellularity as compared to simple incisions and a larger area of scar formation (mean width 3.5 mm). The extent of scar formation and cellularity in incisions exposed to native fatty acids did not significantly differ from that found in incisions with saline exposure (mean width 2.0 mm). However, the extent of scar formation as well as cellularity were reduced when incisions were followed by cauterization and exposure of native fatty acids (mean width 2.5 mm). Exposure of incision wounds to nitrated fatty acids significantly reduced scar formation as compared to saline exposure (mean width 1.1 mm), while exhibiting higher cellularity at the same time. The same holds true for wounds with additional cauterization and exposure to nitrated fatty acids (mean width 1.6 mm).
Conclusions: Nitrated fatty acids reduce fibrotic scar areas after surgical skin incision und suturing when applied to the wound margins. This effect is even more pronounced when the wound margins are additionally traumatized by cauterization.

Nitro fatty acids tested

| 1 | 2 | 3 | 4 | 5 | 6 | 7 | 8 | 9 | 10 | 11 | 12 | 13 | 14 | 15 | 16 | 17 | 18 | 19 | 20 | 21 | 22 |
|---|---|---|---|---|---|---|---|---|---|---|---|---|---|---|---|---|---|---|---|---|---|
| ++ | +++ | ++ | + | 0 | ++ | 0 | + | + | ++ | + | 0 | + | + | + | + | + | + | + | + | ++ | ++ |

0 = not superior to native FA (control); + = Superior to native FA; ++ superior to both; +++ outstanding effect; ND = not determined

### Example 19

### Investigation to Evaluate the Effects Nitrocarboxylic Acids on Tissue Damage Due to Barotrauma Ex Vivo

In order to determine the effects of nitrated fatty acids on mechanical trauma to tracheal cells, an ex vivo model was established. Tracheas of adult Wistar rats sacrificed with thiopental given intraabdominally were carefully resected (harvested). Whole (intact) tracheas were cultured in DMEM 10 (Sigma) supplemented with antibiotic/antimycotic drugs for 48 hours at 37° C. Tracheas were cut into five pieces of equal size. One piece was immediately analyzed, two were bathed in 0.9% saline, one was bathed in a solution containing native fatty acid in SDS (1%), and one was bathed in a solution containing nitro fatty acid, respectively, for 15 minutes each. The inner diameters of the tracheal rings were measured. A non-compliant balloon catheter for use in vascular interventions was chosen, so that the nominal balloon diameter was 15-20% larger than that of the trachea. One tracheal ring pretreated with 0.9% saline was left untreated; the other prepared tracheal rings were mounted on the balloon catheter, which was inflated to a pressure of 4 atm thereafter. Balloons were kept inflated for 4 hours, while being positioned in the culture medium. Thereafter, the tracheal rings were further cultivated in separate vials for 24 hours. In a separate set of investigations, sense/antisense ODNs for HO-1 (Invitrogen) directed against the translation initiation codon in the HO-1 cDNA was used to inhibit hemoxydase-1 synthesis. The cells were transfected using the Superfect transfection reagent (Qiagen) before traumatization. In another set of experiments, the HO inhibitor SnPP IX (Porphyrin Products, London, UK) was added to the culture medium 6 hours before traumatization at a dose of 10 micromol.

For analysis, the rings were cut into small strips using a no touch technique. Viability was tested using a MTT assay and apoptosis using a TUNEL assay. Anti-HO-1 antibodies (StressGen, Tebu, Le-Perray-en-Yvelines, France) were measured by Western blot and immunohistochemistry.

Results: A high proportion (>90%) of cells in the tracheal rings cultured ex vivo and then cultured for 36 hours remained viable and exhibited a low frequency of cells undergoing apoptosis (<5%) when resectants and control samples were compared. Mechanical trauma caused a tremendous reduction in viability (<20% as compared to controls) which corresponded to a massive number of cells being apoptotic (60-80%). Pretreatment with native fatty acids had only a slight effect as compared to the control exhibiting a viability of 20-30% of cells and apoptosis in 50-70% of cells. nitrated fatty acids significantly increased cell viability (70-90% as compared to controls) which was paralleled by a significant reduction of apoptotic cells (20-30% as compared to controls).

In further investigations, a moderate increase of HO-1 was found in untreated control samples as compared to controls investigated shortly after resection. Mechanical traumatization of untreated tracheal rings resulted in a significant rise of HO-1 (30-fold) as compared to the cultured controls. Pretreatment with native fatty acids resulted in a slight decrease (25-fold) in the production of HO-1, whereas nitrated fatty acids led to a slightly greater increase of HO-1 (38-fold). Both the transfection of cells and addition of the HO-1 inhibitor reduced the HO-1 production to the lower detection limit or complete absence in untreated controls as well as in samples treated with native fatty acids or nitrated fatty acids. In samples with blocked HO-1 production, traumatization reduced viability to a higher extent compared to no blockage (0-10% viable cells) and resulted in a higher rate of apoptosis (90-100%). Native fatty acids attenuated this finding, with 10-20% of cells being viable and 80-90% of cells being apoptotic. In contrast, in cells with blocked HO-1 synthesis, nitrated fatty acids led to an almost identical finding of cell viability (60-80%) and apoptosis rate (20-30%) compared to samples without HO-1 inhibition.

Nitro fatty acids tested

| 1 | 2 | 3 | 4 | 5 | 6 | 7 | 8 | 9 | 10 | 11 | 12 | 13 | 14 | 15 | 16 | 17 | 18 | 19 | 20 | 21 | 22 |
|---|---|---|---|---|---|---|---|---|---|---|---|---|---|---|---|---|---|---|---|---|---|
| ++ | + | + | ND | ND | + | ND | + | ND | ++ | + | ND | ND | + | + | ND | ND | ND | ND | ND | ND | + |

0 = not superior to native FA (control); + = Superior to native FA; ++ superior to both; +++ outstanding effect; ND = not determined

Conclusions: Mechanical traumatization of tracheal tissue results in a high rate of cell death. Pretreatment of tracheal tissue with native fatty acids exhibited a modest attenuation of cell death. Nitrated fatty acids, however, significantly reduce the deleterious effects of traumatization. While the traumatization-induced HO-1 production seems to play a role in attenuation of traumatization-induced cell death in the control group and in samples pretreated with native fatty acids, this was not the case when samples are pretreated with nitrated fatty acids. Therefore, nitrated fatty acids exert their cell protective effects on traumatized tracheal cells via a HO-1 independent mechanism.

1. Medical device coated with at least one nitrocarboxylic acid of the general formula (X) 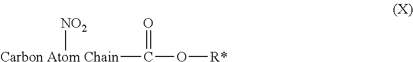
wherein
O—R* represents OH, polyethylene glycolyl, polypropylene glycolyl, cholesteroyl, phytosteroyl, ergosteroyl, coenzyme A or an alkoxy group consisting of 1 to 10 carbon atoms, wherein this alkoxy group may contain one or more double and/or one or more triple bonds and/or may be substituted by one or more nitro groups and/or one or more substituents S¹-S²⁰,
carbon atom chain refers to an alkyl chain to which at least one nitro group is attached consisting of 1 to 40 carbon atoms, wherein this alkyl chain may contain one or more double and/or one or more triple bonds and may be cyclic and/or may be substituted by one or more nitro groups and/or one or more substituents S¹-S²⁰,
S¹-S²⁰ represent independently of each other OH, OP(O)(OH)₂, —P(O)(OH)₂, —P(O)(OCH₃)₂, —OCH₃, —OC₂H₅, —OC₃H₇, —O-cyclo-C₃H₅, —OCH(CH₃)₂, —OC(CH₃)₃, —OC₄H₉, —OPh, —OCH₂Ph, —OCPh₃, —SH, —SCH₃, —SC₂H₅, —F, —Cl, —Br, —I, —CN, —OCN, —NCO, —SCN, —NCS, —CHO, —COCH₃, —COC₂H₅, —COC₃H₇, —CO-cyclo-C₃H₅, —COCH(CH₃)₂, —COC(CH₃)₃, —COOH, —COOCH₃, —COOC₂H₅, —COOC₃H₇, —COO-cyclo-C₃H₅, —COOCH(CH₃)₂, —COOC(CH₃)₃, —OOC—CH₃, —OOC—C₂H₅, —OOC—C₃H₇, —OOC-cyclo-C₃H₅, —OOC—CH(CH₃)₂, —OOC—C(CH₃)₃, —CONH₂, —CONHCH₃, —CONHC₂H₅, —CONHC₃H₇, —CON(CH₃)₂, —CON(C₂H₅)₂, —CON(C₃H₇)₂, —NH₂, —NHCH₃, —NHC₂H₅, —NHC₃H₇, —NH-cyclo-C₃H₅, —NHCH(CH₃)₂, —NHC(CH₃)₃, —N(CH₃)₂, —N(C₂H₅)₂, —N(C₃H₇)₂, —N(cyclo-C₃H₅)₂, —N[CH(CH₃)₂]₂, —N[C(CH₃)₃]₂, —SOCH₃, —SOC₂H₅, —SOC₃H₇, —SO₂CH₃, —SO₂C₂H₅, —SO₂C₃H₇, —SO₃H, —SO₃CH₃, —SO₃C₂H₅, —SO₃C₃H₇, —OCF₃, —OC₂F₅, —O—COOCH₃, —O—COOC₂H₅, —O—COOC₃H₇, —O—COO-Cyclo-C₃H₅, —O—COOCH(CH₃)₂, —O—COOC(CH₃)₃, —NH—CO—NH₂, —NH—CO—NHCH₃, —NH—CO—NHC₂H₅, —NH—CO—N(CH₃)₂, —NH—CO—N(C₂H₅)₂, —O—CO—NH₂, —O—CO—NHCH₃, —O—CO—NHC₂H₅, —O—CO—NHC₃H₇, —O—CO—N(CH₃)₂, —O—CO—N(C₂H₅)₂, —O—CO—OCH₃, —O—CO—OC₂H₅, —O—CO—OC₃H₇, —O—CO—O-cyclo-C₃H₅, —O—CO—OCH(CH₃)₂, —O—CO—OC(CH₃)₃, —CH₂F, —CHF₂, —CF₃, —CH₂Cl, —CH₂Br, —CH₂I, —CH₂—CH₂F, —CH₂—CHF₂, —CH₂—CF₃, —CH₂—CH₂Cl, —CH₂—CH₂Br, —CH₂—CH₂₁, —CH₃, —C₂H₅, —C₃H₇, -cyclo-C₃H₅, —CH(CH₃)₂, —C(CH₃)₃, —C₄H₉, —CH₂—CH(CH₃)₂, —CH(CH₃)—C₂H₅, —C₅H₁₁, -Ph, —CH₂-Ph, —CPh₃, —CH═CH₂, —CH₂—CH═CH₂, —C(CH₃)═CH₂, —CH═CH—CH₃, —C₂H₄—CH═CH₂, —CH═C(CH₃)₂, —C≡CH, —C═C—CH₃, —CH₂—C≡CH, —P(O)(OC₂H₅)₂, cholesteryl, nucleotides, lipoamines, dihydrolipoamines, lysobiphospatidic acid, anandamide, long chain N-acyl-ethanolamide, sn-1 substituents with glycerol or diglycerol, sn-2 substituents with glycerol or diglycerol, sn-3 substituents, ceramide, sphingosine, ganglioside, galactosylceramide or aminoethylphosphonic acid.

2. Medical device according to claim 1, wherein the at least one nitrocarboxylic acid is selected from 12-nitro-linoleic acid, 9-nitro cis-oleic acid, 10-nitro-cis-linoleic acid, 10-nitro-cis-oleic acid, 5-nitro-eicosatrienoic acid, 16-nitro-all-cis-4,7,10,13,16-docosapentaenoic acid, 9-nitro-all-cis-9-12,15-octadecatrienoic acid, 14-nitro-all-cis-7,10,13,16,19-docosapentaenoic acid, 15-nitro-cis-15-tetracosenoic acid, 9-nitro-trans-oleic acid, 9,10-nitro-cis-oleic acid, 13-nitro-octadeca-9,11,13-trienoic acid, 10-nitro-trans-oleic acid, 9-nitro-cis-hexadecenoic acid, 11-nitro-5,8,11,14-eicosatrienoic acid, 9,10-nitro-trans-oleic acid, 9-nitro-9-trans-hexadecenoic acid, 13-nitro-cis-13-docosenoic acid, 8,14-nitro-cis-5,8,11,14-eicosatetraenoic acid, 4,16-nitro-docosahexaenoic acid, 9-nitro-cis-6,9,12-octadecatrienoic acid, 6-nitro-cis-6-octadecenoic acid, 11-nitro-cis-5,8,11,14-eicosatetraenoic acid and combinations thereof.

3. Medical device according to claim 1, wherein the medical device is selected from the group comprising or consisting of tissue replacement implants, breast implants, soft implants, autologous implants, joint implants, cartilage implants, natural or artificial tissue implants and grafts, autogenous tissue implants, intraocular lenses, surgical adhesion barriers, nerve regeneration conduits, birth control devices, shunts, tissue scaffolds; tissue-related materials including small intestinal submucosal matrices, dental devices and dental implants, drug infusion tubes, cuffs, drainage devices, tubes, surgical meshes, ligatures, sutures, staples, patches, slings, foams, pellicles, films, implantable electrical stimulators, pumps, ports, reservoirs, catheters for injection or stimulation or sensing, wound coatings, suture material, surgical instruments such as scalpels, lancets, scissors, forceps or hooks, clinical gloves, injection needles, endoprotheses and exoprotheses as well as osteosynthetic materials.

4. Medical device according to claim 3, wherein the soft implant is selected from a saline breast implant, silicone breast implant, triglyceride-filled breast implant, chin and mandibular implant, nasal implant, cheek implant, lip implant, and other facial implant, pectoral and chest implant, malar and submalar implant, and buttocks implant.

5. Medical device according to claim 3, wherein the surgical mesh or artificial tissue is produced from synthetic or natural polymers like polypropylene, polyester, polytetrafluoroethylene, PETNF or PTFENF or Dacron.

6. Medical device according to claim 1 wherein the nitrocarboxylic acid is derived from hexanoic acid, octanoic acid, decanoic acid, dodecanoic acid, tetradecanoic acid, hexadecanoic acid, heptadecanoic acid, octadecanoic acid, eicosanoic acid, docosanoic acid, tetracosanoic acid, cis-9-tetradecenoic acid, cis-9-hexadecenoic acid, cis-6-octadecenoic acid, cis-9-octadecenoic acid, cis-11-octadecenoic acid, cis-9-eicosenoic acid, cis-11-eicosenoic acid, cis-13-docosenoic acid, cis-15-tetracosenoic acid, t9-octadecenoic acid, t11-octadecenoic acid, t3-hexadecenoic acid, 9,12-octadecadienoic acid, 6,9,12-octadecatrienoic acid, 8,11,14-eicosatrienoic acid, 5,8,11,14-eicosatetraenoic acid, 7,10,13,16-docosatetraenoic acid, 4,7,10,13,16-docosapentaenoic acid, 9,12,15-octadecatrienoic acid, 6,9,12,15-octadecatetraenoic acid, 8,11,14,17-eicosatetraenoic acid, 5,8,11,14,17-eicosapentaenoic acid, 7,10,13,16,19-docosapentaenoic acid, 4,7,10,13,16,19-docosahexaenoic acid, 5,8,11-eicosatrienoic acid, 9c 11t 13t eleostearic acid, 8t 10t 12c calendic acid, 9c 11t 13c catalpic acid, 4, 7, 9, 11, 13, 16, 19 docosaheptadecanoic acid, taxoleic acid, pinolenic acid, sciadonic acid, 6-octadecynoic acid, t11-octadecen-9-ynoic acid, 9-octadecynoic acid, 6-octadecen-9-ynoic acid, t10-heptadecen-8-ynoic acid, 9-octadecen-12-ynoic acid, t7,t11-octadecadiene-9-ynoic acid, t8,t10-octadecadiene-12-ynoic acid, 5,8,11,14-eicosatetraynoic acid, retinoic acid, isopalmitic acid, pristanic acid, phytanic acid, 11,12-methyleneoctadecanoic acid, 9,10-methylenhexadecanoic acid, coronaric acid, (R,S)-lipoic acid, (S)-lipoic acid, (R)-lipoic acid, 6,8-bis(methylsulfanyl)-octanoic acid, 4,6-bis(methylsulfanyl)-hexanoic acid, 2,4-bis(methylsulfanyl)-butanoic acid, 1,2-dithiolane carboxylic acid, (R,S)-6,8-dithiane octanoic acid, (R)-6,8-dithiane octanoic acid, (S)-6,8-dithiane octanoic acid, tariric acid, santalbic acid, stearolic acid, 6,9-octadecenynoic acid, pyrulic acid, crepenynic acid, heisteric acid, t8,t10-octadecadiene-12-inoic acid, ETYA, cerebronic acid, hydroxynervonic acid, ricinoleic acid, lesquerolic acid, brassylic acid and thapsic acid.

7. Medical device according to claim 1, wherein the medical device is covered with a layer containing the at least one nitrocarboxylic acid applied to the surface to the medical implant by a pipetting method, spray method, dipping method or vapor deposition method.

8. Use of a nitrocarboxylic acid of the general formula (X) 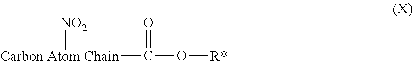
wherein
O—R* represents OH, polyethylene glycolyl, polypropylene glycolyl, cholesteroyl, phytosteroyl, ergosteroyl, coenzyme A or an alkoxy group consisting of 1 to 10 carbon atoms, wherein this alkoxy group may contain one or more double and/or one or more triple bonds and/or may be substituted by one or more nitro groups and/or one or more substituents S¹-S²⁰,
carbon atom chain refers to an alkyl chain to which at least one nitro group is attached consisting of 1 to 40 carbon atoms, wherein this alkyl chain may contain one or more double and/or one or more triple bonds and/or may be substituted by one or more nitro groups and/or one or more substituents S¹-S²⁰,
S¹-S²⁰ represent independently of each other —OH, —OP(O)(OH)₂, —P(O)(OH)₂, —P(O)(OCH₃)₂, —OCH₃, —OC₂H₅, —OC₃H₇, —O-cyclo-C₃—H₅, —OCH(CH₃)₂, —OC(CH₃)₃, —OC₄H₉, —OPh, —OCH₂-Ph, —OCPh₃, —SH, —SCH₃, —SC₂H₅, —F, —Cl, —Br, —I, —CN, —OCN, —NCO, —SCN, —NCS, —CHO, —COCH₃, —COC₂H₅, —COC₃H₇, —CO-cyclo-C₃H₅, —COCH(CH₃)₂, COC(CH₃)₃, —COOH, —COOCH₃, —COOC₂H₅, —COOC₃H₇, —COO-cyclo-C₃H₅, —COOCH(CH₃)₂, —COOC(CH₃)₃, —OOC—CH₃, —OOC—C₂H₅, —OOC—C₃H₇, —OOC-cyclo-C₃H₅, —OOC—CH(CH₃)₂, —OOC—C(CH₃)₃, —CONH₂, —CONHCH₃, —CONHC₂H₅, —CONHC₃H₇, —CON(CH₃)₂, —CON(C₂H₅)₂, —CON(C₃H₇)₂, —NH₂, —NHCH₃, —NHC₂H₅, —NHC₃H₇, —NH-cyclo-C₃H₅, —NHCH(CH₃)₂, —NHC(CH₃)₃, —N(CH₃)₂, —N(C₂H₅)₂, —N(C₃H₇)₂, —N(cyclo-C₃H₅)₂, —N[CH(CH₃)₂]₂, —N[C(CH₃)₃]₂, —SOCH₃, —SOC₂H₅, —SOC₃H₇, —SO₂CH₃, —SO₂C₂H₅, —SO₂C₃H₇, —SO₃H, —SO₃CH₃, —SO₃C₂H₅, —SO₃C₃H₇, —OCF₃, —OC₂F₅, —O—COOCH₃, —O—COOC₂H₅, —O—COOC₃H₇, —O—COO-cyclo-C₃H₅, —O—COOCH(CH₃)₂, —O—COOC(CH₃)₃, —NH—CO—NH₂, —NH—CO—NHCH₃, —NH—CO—NHC₂H₅, —NH—CO—N(CH₃)₂, —NH—CO—N(C₂H₅)₂, —O—CO—NH₂, —O—CO—NHCH₃, —O—CO—NHC₂H₅, —O—CO—NHC₃H₇, —O—CO—N(CH₃)₂, —O—CO—N(C₂H₅)₂, —O—CO—OCH₃, —O—CO—OC₂H₅, —O—CO—OC₃H₇, —O—CO—O-cyclo-C₃H₅, —O—CO—OCH(CH₃)₂, —O—CO—OC(CH₃)₃, —CH₂F, —CHF₂, —CF₃, —CH₂Cl, —CH₂Br, —CH₂I, —CH₂—CH₂F, —CH₂—CHF₂, —CH₂—CF₃, —CH₂—CH₂Cl, —CH₂—CH₂Br, —CH₂—CH₂I, —CH₃, —C₂H₅, —C₃H₇, -cyclo-C₃H₅, —CH(CH₃)₂, —C(CH₃)₃, —C₄H₉, —CH₂—CH(CH₃)₂, —CH(CH₃)—C₂H₅, —C₅H₁₁, -Ph, —CH₂-Ph, —CPh₃, —CH═CH₂, —CH₂—CH═CH₂, —C(CH₃)═CH₂, —CH═CH—CH₃, —C₂H₄—CH═CH₂, —CH═C(CH₃)₂, C≡CH, C≡C—CH₃, —CH₂—C≡CH, —P(O)(OC₂H₅)₂, cholesteryl, nucleotides, lipoamines, dihydrolipoamines, lysobiphospatidic acid, anandamide, long chain N-acyl-ethanolamide, sn-1 substituents with glycerol or diglycerol, sn-2 substituents with glycerol or diglycerol, sn-3 substituents, ceramide, sphingosine, ganglioside, galactosylceramide or aminoethylphosphonic acid for the manufacture of a pharmaceutical composition for the treatment or prophylaxis of a disease or a state displaying an aggressive healing response of tissues, cells or organelles which is not due to a genuine inflammation.

9. Use of a nitrocarboxylic acid according to claim 8, wherein the nitrocarboxylic acid is derived from hexanoic acid, octanoic acid, decanoic acid, dodecanoic acid, tetradecanoic acid, hexadecanoic acid, heptadecanoic acid, octadecanoic acid, eicosanoic acid, docosanoic acid, tetracosanoic acid, cis-9-tetradecenoic acid, cis-9-hexadecenoic acid, cis-6-octadecenoic acid, cis-9-octadecenoic acid, cis-11-octadecenoic acid, cis-9-eicosenoic acid, cis-11-eicosenoic acid, cis-13-docosenoic acid, cis-15-tetracosenoic acid, t9-octadecenoic acid, t11-octadecenoic acid, t3-hexadecenoic acid, 9,12-octadecadienoic acid, 6,9,12-octadecatrienoic acid, 8,11,14-eicosatrienoic acid, 5,8,11,14-eicosatetraenoic acid, 7,10,13,16-docosatetraenoic acid, 4,7,10,13,16-docosapentaenoic acid, 9,12,15-octadecatrienoic acid, 6,9,12,15-octadecatetraenoic acid, 8,11,14,17-eicosatetraenoic acid, 5,8,11,14,17-eicosapentaenoic acid, 7,10,13,16,19-docosapentaenoic acid, 4,7,10,13,16,19-docosahexaenoic acid, 5,8,11-eicosatrienoic acid, 9c 11t 13t eleostearic acid, 8t 10t 12c calendic acid, 9c 11t 13c catalpic acid, 4, 7, 9, 11, 13, 16, 19 docosaheptadecanoic acid, taxoleic acid, pinolenic acid, sciadonic acid, 6-octadecynoic acid, t11-octadecen-9-ynoic acid, 9-octadecynoic acid, 6-octadecen-9-ynoic acid, t10-heptadecen-8-ynoic acid, 9-octadecen-12-ynoic acid, t7,t11-octadecadiene-9-ynoic acid, t8,t10-octadecadiene-12-ynoic acid, 5,8,11,14-eicosatetraynoic acid, retinoic acid, isopalmitic acid, pristanic acid, phytanic acid, 11,12-methyleneoctadecanoic acid, 9,10-methylenhexadecanoic acid, coronaric acid, (R,S)-lipoic acid, (S)-lipoic acid, (R)-lipoic acid, 6,8-bis(methylsulfanyl)-octanoic acid, 4,6-bis(methylsulfanyl)-hexanoic acid, 2,4-bis(methylsulfanyl)-butanoic acid, 1,2-dithiolane carboxylic acid, (R,S)-6,8-dithiane octanoic acid, (R)-6,8-dithiane octanoic acid, (S)-6,8-dithiane octanoic acid, tariric acid, santalbic acid, stearolic acid, 6,9-octadecenynoic acid, pyrulic acid, crepenynic acid, heisteric acid, t8,t10-octadecadiene-12-inoic acid, ETYA, cerebronic acid, hydroxynervonic acid, ricinoleic acid, lesquerolic acid, brassylic acid and thapsic acid.

10. Use of a nitrocarboxylic acid according to claim 8, wherein medical treatment is associated with potential irritation or injury of cells, organs or tissues by physical, chemical, or electrical irritation displaying an aggressive healing response as derived from surgical, plastic or cosmetic procedures causing injuries, wherein said irritation or injury is selected from cut, tear, dissection, resection, suture, wound closure, debridgement, cauterization, suction, drainage, implantation, grafting, fracture, osteosynthesis, radiation, laser or tissue welding.

11. Use of a nitrocarboxylic acid according to claim 8 for the protection of tissues, in situ or ex vivo organs, or transplants from cold preservation impairment.

12. Use of a nitrocarboxylic acid according to claim 8 for the stabilization of membrane functions in cells and organelles for the prophylaxis or treatment of diseases or states such as acute or chronic pain, hypersensitivity syndrome, neuropathic pain, atopies such as urticaria, allergic rhinitis and hay fever, enteropathies such as tropical sprue or coeliac disease.

13. Use of a nitrocarboxylic acid according to claim 8, wherein said disease or state displaying an aggressive healing response results from an exogenous irritation, wounding or trauma, wherein the disease or state in which such an exogenous irritation, wounding or trauma occurs is selected from burn, chemical burn, alkali burn, burning, hypothermia, frostbite, cauterization, granuloma, necrosis, ulcer, fracture, foreign body reaction, cut, scratch, laceration, bruise, tear, contusion, fissuring, burst,
or from an endogenous irritation or stimulation by acute or chronical physical, chemical or electrical means wherein the disease or state in which such an endogenous irritation or stimulation occurs is selected from fascitis, tendonitis, neuropathy or prostate hypertrophy.

14. Use of a nitrocarboxylic acid according to claim 8, wherein said disease or state displaying an aggressive healing response affects the properties, function and reactivity of cell, organelle or plasma membranes and results from chronic or acute irritation or stimulation, wherein the chronic or acute irritation or stimulation is selected from physical trauma, chemical trauma, electrical trauma, immunological biomolecules, malnutrition and toxins or poisons, wherein the diseases caused by said toxins or poisons are selected from neuropathy, acute pain, chronic pain, hypersensitivity syndrome, neuropathic pain, burning feet syndrome, induratio fibroplastica penis and Sudeck's atrophy.

15. Use of a nitrocarboxylic acid according to claim 8, wherein the disease or state displaying an aggressive healing response results secondary to an immunological process from a disease with an additional inflammatory component which is not a genuine inflammatory disease, wherein such a disease with an additional inflammatory component is a osteomyelofibrosis, chronic polyarthritis, atrophia of mucuous tissues or epidermis, dermatitis ulcerosa, connective tissue diseases such dermatomyositis, chronic vasculitis, polyarteritis nodosa, hypersensitivity angiitis, Wegener's granulomatosis, non-tropical sprue, arthropathy, peri-arthropathy, fibromyalgia, meralgia paresthetica, carpal tunnel syndrome and nerve compression syndrome,
or from an immunological process or disease which is not a genuine immunopathy, wherein such immunological process or disease is selected from enteropathies such as tropical sprue or coeliac disease, or from bronchiectasis, emphysema, chronic obstructive pulmonary disease (COPD), dermatoses such as atrophic contact dermatosis, or from gouty arthritis, osteoarthrosis, degenerative arthrotic conditions, toxic shock syndrome, amyolidosis, dermatitis ulcerosa, nephrosclerosis, cystic fibrosis, atopic dermatose, atrophy of mucuous tissue or epidermis, connective tissue diseases such as Sharp syndrome and dermatomyositis, aphthous ulcer, Stevens-Johnson syndrome, toxic epidermal necrolysis.

16. Medical device according to claim 2, wherein the medical device is selected from the group comprising or consisting of tissue replacement implants, breast implants, soft implants, autologous implants, joint implants, cartilage implants, natural or artificial tissue implants and grafts, autogenous tissue implants, intraocular lenses, surgical adhesion barriers, nerve regeneration conduits, birth control devices, shunts, tissue scaffolds; tissue-related materials including small intestinal submucosal matrices, dental devices and dental implants, drug infusion tubes, cuffs, drainage devices, tubes, surgical meshes, ligatures, sutures, staples, patches, slings, foams, pellicles, films, implantable electrical stimulators, pumps, ports, reservoirs, catheters for injection or stimulation or sensing, wound coatings, suture material, surgical instruments such as scalpels, lancets, scissors, forceps or hooks, clinical gloves, injection needles, endoprotheses and exoprotheses as well as osteosynthetic materials.

17. Medical device according to claim 2 wherein the nitrocarboxylic acid is derived from hexanoic acid, octanoic acid, decanoic acid, dodecanoic acid, tetradecanoic acid, hexadecanoic acid, heptadecanoic acid, octadecanoic acid, eicosanoic acid, docosanoic acid, tetracosanoic acid, cis-9-tetradecenoic acid, cis-9-hexadecenoic acid, cis-6-octadecenoic acid, cis-9-octadecenoic acid, cis-11-octadecenoic acid, cis-9-eicosenoic acid, cis-11-eicosenoic acid, cis-13-docosenoic acid, cis-15-tetracosenoic acid, t9-octadecenoic acid, t11-octadecenoic acid, t3-hexadecenoic acid, 9,12-octadecadienoic acid, 6,9,12-octadecatrienoic acid, 8,11,14-eicosatrienoic acid, 5,8,11,14-eicosatetraenoic acid, 7,10,13,16-docosatetraenoic acid, 4,7,10,13,16-docosapentaenoic acid, 9,12,15-octadecatrienoic acid, 6,9,12,15-octadecatetraenoic acid, 8,11,14,17-eicosatetraenoic acid, 5,8,11,14,17-eicosapentaenoic acid, 7,10,13,16,19-docosapentaenoic acid, 4,7,10,13,16,19-docosahexaenoic acid, 5,8,11-eicosatrienoic acid, 9c 11t 13t eleostearic acid, 8t 10t 12c calendic acid, 9c 11t 13c catalpic acid, 4, 7, 9, 11, 13, 16, 19 docosaheptadecanoic acid, taxoleic acid, pinolenic acid, sciadonic acid, 6-octadecynoic acid, t11-octadecen-9-ynoic acid, 9-octadecynoic acid, 6-octadecen-9-ynoic acid, t10-heptadecen-8-ynoic acid, 9-octadecen-12-ynoic acid, t7,t11-octadecadiene-9-ynoic acid, t8,t10-octadecadiene-12-ynoic acid, 5,8,11,14-eicosatetraynoic acid, retinoic acid, isopalmitic acid, pristanic acid, phytanic acid, 11,12-methyleneoctadecanoic acid, 9,10-methylenhexadecanoic acid, coronaric acid, (R,S)-lipoic acid, (S)-lipoic acid, (R)-lipoic acid, 6,8-bis(methylsulfanyl)-octanoic acid, 4,6-bis(methylsulfanyl)-hexanoic acid, 2,4-bis(methylsulfanyl)-butanoic acid, 1,2-dithiolane carboxylic acid, (R,S)-6,8-dithiane octanoic acid, (R)-6,8-dithiane octanoic acid, (S)-6,8-dithiane octanoic acid, tariric acid, santalbic acid, stearolic acid, 6,9-octadecenynoic acid, pyrulic acid, crepenynic acid, heisteric acid, t8,t10-octadecadiene-12-inoic acid, ETYA, cerebronic acid, hydroxynervonic acid, ricinoleic acid, lesquerolic acid, brassylic acid and thapsic acid.

18. Medical device according to claim 2, wherein the medical device is covered with a layer containing the at least one nitrocarboxylic acid applied to the surface to the medical implant by a pipetting method, spray method, dipping method or vapor deposition method.
